(19) United States
(12) Patent Application Publication
(10) Pub. No.: US 2005/0019810 A1
(43) Pub. Date: Jan. 27, 2005

(54) Novel genes encoding proteins having prognostic, diagnostic, preventive, therapeutic and other uses

(75) Inventors: Douglas A. Holtzman, Jamaica Plain, MA (US); Andrew D.J. Goodearl, Natick, MA (US); Sean A. McCarthy, San Diego, CA (US)

Correspondence Address: MILLENNIUM PHARMACEUTICALS, INC., 40 Landsdowne Street, Cambridge, MA, 02139, US

(73) Assignee: Millennium Pharmaceuticals, Inc.

(21) Appl. No.: 10/900,926

(22) Filed: Jul. 28, 2004

Related U.S. Application Data
(63) Continuation of application No. 10/269,353, filed on Oct. 11, 2002.
Application No. 10/269,353 is a continuation of application No. 09/790,264, filed on Feb. 21, 2001, now abandoned.
Application No. 09/790,264 is a continuation-in-part of application No. 09/065,661, filed on Apr. 23, 1998, now abandoned.
Application No. 09/790,264 is a continuation-in-part of application No. 09/298,531, filed on Apr. 23, 1999, now abandoned.
Application No. 09/298,531 is a continuation-in-part of application No. 09/065,363, filed on Apr. 23, 1998, now abandoned.
Application No. 09/790,264 is a continuation-in-part of application No. 09/337,930, filed on Jun. 22, 1999, now abandoned.
Application No. 09/337,930 is a continuation-in-part of application No. 09/102,705, filed on Jun. 22, 1998, now abandoned.
Application No. 09/790,264 is a continuation-in-part of application No. 09/363,630, filed on Jul. 29, 1999, now abandoned.
Application No. 09/363,630 is a continuation-in-part of application No. 09/124,538, filed on Jul. 29, 1998, now abandoned.

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

The invention provides isolated nucleic acids encoding a variety of proteins having diagnostic, preventive, therapeutic, and other uses. These nucleic and proteins are useful for diagnosis, prevention, and therapy of a number of human and other animal disorders. The invention also provides antisense nucleic acid molecules, expression vectors containing the nucleic acid molecules of the invention, host cells into which the expression vectors have been introduced, and non-human transgenic animals in which a nucleic acid molecule of the invention has been introduced or disrupted. The invention still further provides isolated polypeptides, fusion polypeptides, antigenic peptides and antibodies. Diagnostic, screening, and therapeutic methods using compositions of the invention are also provided. The nucleic acids and polypeptides of the present invention are useful as modulating agents in regulating a variety of cellular processes.

### TECHNICAL FIELD

This invention relates to polypeptides and the genes encoding them.

### CROSS REFERENCE TO RELATED APPLICATIONS

This application is a continuation-in-part (and claims the benefit of priority under 35 USC 120) of the following applications:
U.S. application Ser. No. 09/065,661 (filed Apr. 23, 1998).
U.S. application Ser. No. 09/298,531 (filed Apr. 23, 1999), which is a continuation-in-part of U.S. application Ser. No. 09/065,363 (filed Apr. 23, 1998).
U.S. application Ser. No. 09/337,930 (filed Jun. 22, 1999), which is a continuation-in-part of U.S. application Ser. No. 09/102,705 (filed Jun. 22, 1998).
U.S. application Ser. No. 09/363,630 (filed Jul. 29, 1999), which is a continuation-in-part of U.S. application Ser. No. 09/124,538 (filed Jul. 29, 1998).

The disclosures of the prior applications are considered part of (and are incorporated by reference in) the disclosure of this application.

### BACKGROUND OF THE INVENTION

Many membrane-associated and secreted proteins, for example, cytokines, play a vital role in the regulation of cell growth, cell differentiation, and a variety of specific cellular responses. A number of medically useful proteins, including erythropoietin, granulocyte-macrophage colony stimulating factor, human growth hormone, and various interleukins, are secreted proteins. Thus, an important goal in the design and development of new therapies is the identification and characterization of membrane-associated and secreted proteins and the genes which encode them.

Many membrane-associated proteins are receptors which bind a ligand and transduce an intracellular signal, leading to a variety of cellular responses. The identification and characterization of such a receptor enables one to identify both the ligands which bind to the receptor and the intracellular molecules and signal transduction pathways associated with the receptor, permitting one to identify or design modulators of receptor activity, e.g., receptor agonists or antagonists and modulators of signal transduction.

Within tissues, an organized network of proteins and polysaccharides is associated with cell membranes. This network, known as the extracellular matrix, functions to provide structural integrity to tissues. Additionally, the extracellular matrix regulates the development, proliferation, migration and function of cells that contact it. Important to its function is the tightly regulated control of its degradation and resynthesis. Such degradation occurs during a variety of processes, including the involution of the uterus following childbirth, involution of the mammary gland following completion of lactation, migration of white blood cells through vascular basal lamina following tissue injury or infection, migration of cancer cells in metastasis, angiogenesis and cell proliferation.

These processes are controlled by the cooperative action of proteases and specific protease inhibitors. Protease inhibitors are secreted into blood, mucous, salivary gland secretions, tear fluid and skin and can act systemically or locally. Their secretion by cells at sites of protease action may help localize degradation by proteases to specific areas within affected tissues. A number of protease inhibitors are members of the “four-disulfide core” family of proteins. The conserved pattern of cysteines found in members of this family predicts a related tertiary structure and is suggestive of protease inhibitory activity.

One group of locally-acting protease inhibitors within the “four-disulfide core” family are the anti-leukoproteinases. These protease inhibitors have been shown to be involved in a variety of cell processes and disorders. For example, rat Westmead DMBA8 nonmetastatic cDNA 1, WDNM-1, (Dear & Kefford (1991) Biochem. & Biophys. Res. Comm. 176:247-254) is downregulated in metastatic versus non-metastatic rat mammary adenocarcinoma and may function as a metastasis inhibitor (Dear et al. (1988) Cancer Res. 48:5203-5209). Likewise, murine WDNM-1 has been identified as a genetic marker for murine mammary tumors transformed by the oncogenes neu or ras (Morrison & Leder (1994) Oncogene 9:3417-3426). Additionally, human anti-leukoproteinase has been shown to promote hematopoiesis by inhibiting degradation of cytokines, growth factor receptors and other proteins involved in blood cell growth and differentiation (Goselink et al. (1996) J. Exp. Med. 184:1305-1313), while experiments with porcine anti-leukoproteinase demonstrate a function in the maintenance and progression of pregnancy (Simmen et al. (1991) Biol. Reprod. 44:191-200). In rats, anti-leukoproteinase has been shown to be depleted in arthritic cartilage (Burkhardt et al. (1997) J. Rheumatol. 24:1145-1154.

A murine anti-leukoproteinase, (“MALP”), also known as a secretory leukocyte protease inhibitor (“SLPI”), inhibits bacterial lipopolysaccharide and may be useful in the treatment of septic shock (Jin et al. (1997) Cell 88:417-26). SLPI has also been implicated in chronic respiratory diseases such as chronic bronchitis, emphysema, cystic fibrosis (Mitsuhashi, et al. (1997) J. Pharmacol. Exp. Ther. 282:1005-1010) and asthma (Fath et al. (1998) J. Biol. Chem. 273:13563-13569). Additionally, SLPI may also have a broad spectrum antibiotic activity that includes antiretroviral, bactericidal, and antifungal activity (Tomee et al. (1998) Thorax 53:114-116).

A human skin-derived anti-leukoproteinase (“SKALP”) is elevated in psoriasis and wound healing (Schalkwijk et al. (1991) Biochem. Biophys. Acta 1096:148-154) and is differentially expressed in epidermal carcinomas (Alkemade et al. (1993) Am. J. Path. 143:1679-1687). Other anti-leukoproteinases have been identified, including one from trout (GenBank Accession Number U03890), and it is believed that additional anti-leukoproteinases with different or related functions are yet to be identified. Active peptides derived from anti-leukoproteinases have been proposed as therapies for the treatment of conditions in which anti-leukoproteinases play a role. Thus, these molecules, peptides derived from them, and modulators thereof may have utility in the treatment and prevention of such conditions and as markers for specific disease states.

Cellular interactions with the extracellular matrix are mediated, in part, through the family of cell-surface molecules known as integrins. A subfamily of integrins recognizes and binds to the peptide sequence Arginine-Glycine-Aspartate (RGD) found in extracellular matrix proteins such as fibronectin. The interaction of cells with matrix RGD is important in normal processes such as wound healing, blood clotting and hematopoiesis and plays a role in abnormal states, such as metastasis. Secreted proteins that contain RGD have potential clinical value in modulating these interactions. For example, the disintegrins, a family of secreted snake venom proteins that bind integrins, contain RGD and act as potent platelet aggregation inhibitors (Perutelli (1995) Recenti Progressi in Medicina 86:168-74). Thus, RGD-containing peptides may have utility as antithrombotic agents and in the prevention of arterial thrombosis (Schafer (1996) Am. J. Med. 101:199-209).

### SUMMARY OF THE INVENTION

The present invention is based, at least in part, on the discovery of the following:
A cDNA molecule encoding human T139 (also known as TANGO-139);
A cDNA molecule encoding human T125 (also known as TANGO-125);
A cDNA molecule encoding human T110 (also known as TANGO-110); and
cDNA molecules encoding murine T175 (also known as TANGO-175), human T175, and murine WDNM-2.

The nucleic acids and polypeptides of the present invention are useful as modulating agents in regulating a variety of cellular processes (e.g., cell proliferation and/or cell differentiation). Accordingly, in one aspect, this invention provides isolated nucleic acid molecules encoding T139, T125, T110, T175, and WDNM-2 proteins or biologically active portions thereof, as well as nucleic acid fragments suitable as primers or hybridization probes for the detection of T139, T125, T110, T175 or WDNM-2-encoding nucleic acids.

The present invention is based, at least in part, on the discovery of a gene encoding T139. The T139 polypeptide is predicted to include a signal sequence and possesses several domains (a sperm-coating protein (SCP) domain, a C-type domain, and two epidermal growth factor (EGF)-like domains).

The present invention is based, at least in part, on the discovery of a gene encoding T125, a protein that may be secreted. The T139 polypeptide is predicted to include a signal sequence and possesses two epidermal growth factor (EGF) domains. Unless otherwise specified, “T125” (or “TANGO 125”) is used to refer to all forms of T125 (T125, T125a, T125b, and T125c).

The present invention is based, at least in part, on the discovery of a gene encoding T110, a protein that may be secreted. T110 protein is related to four-joint (fj) protein of Drosophila melanogaster, and is predicted to be a member of the type-II membrance protein superfamily. Such proteins usually employ a transmembrane domain as the internal signal sequence.

The present invention is based, at least in part, on the discovery of a gene encoding T175, a secreted protein that is related to several proteins in the four disulfide core family. The present invention is also based, at least in part, on the discovery of a gene encoding murine WDNM-2, a protein that, like T175, is related to several proteins in the four disulfide core family.

The invention features a nucleic acid molecule which is at least 45% (or 55%, 65%, 75%, 85%, 95%, or 98%) identical to the nucleotide sequence shown in SEQ ID NO:1, or SEQ ID NO:3, or the nucleotide sequence of the cDNA insert of the plasmid deposited with ATCC as Accession Number (the “cDNA of ATCC 98694”), or a complement thereof.

The invention features a nucleic acid molecule which includes a fragment of at least 300 (325, 350, 375, 400, 425, 450, 500, 550, 600, 650, 700, 800, 900, 1000, or 1290) nucleotides of the nucleotide sequence shown in SEQ ID NO:1, or SEQ ID NO:3, or the nucleotide sequence of the cDNA of ATCC 98694, or a complement thereof.

The invention also features a nucleic acid molecule which includes a nucleotide sequence encoding a protein having an amino acid sequence that is at least 45% (or 55%, 65%, 75%, 85%, 95%, or 98%) identical to the amino acid sequence of SEQ ID NO:2, SEQ ID NO:4, or the amino acid sequence encoded by the cDNA of ATCC 98694. In a preferred embodiment, a T139 nucleic acid molecule has the nucleotide sequence shown SEQ ID NO:1, or SEQ ID NO:3, or the nucleotide sequence of the cDNA of ATCC 98694.

Also within the invention is a nucleic acid molecule which encodes a fragment of a polypeptide having the amino acid sequence of SEQ ID NO:2 or SEQ ID NO:4, the fragment including at least 15 (25, 30, 50, 100, 150, 300, or 400) contiguous amino acids of SEQ ID NO:2 or SEQ ID NO:4 or the polypeptide encoded by the cDNA of ATCC 98694.

The invention includes a nucleic acid molecule which encodes a naturally occurring allelic variant of a polypeptide comprising the amino acid sequence of SEQ ID NO:2 or SEQ ID NO:4 or an amino acid sequence encoded by the cDNA of ATCC 98694, wherein the nucleic acid molecule hybridizes to a nucleic acid molecule comprising SEQ ID NO:1 or SEQ ID NO:3 under stringent conditions.

Also within the invention are: an isolated T139 protein having an amino acid sequence that is at least about 65%, preferably 75%, 85%, 95%, or 98% identical to the amino acid sequence of SEQ ID NO:4 (mature human T139) or the amino acid sequence of SEQ ID NO:2 (immature human T139); and an isolated T139 protein having an amino acid sequence that is at least about 85%, 95%, or 98% identical to the SCP-like domain of SEQ ID NO:2 (e.g., about amino acid residues 47 to 190 of SEQ ID NO:2), C-type lectin domain (e.g., about amino acid residues 297 to 412 of SEQ ID NO:2), and EGF-like domains (e.g., about amino acids residues 232 to 260 or 264 to 291 of SEQ ID NO:2).

Also within the invention are: an isolated T139 protein which is encoded by a nucleic acid molecule having a nucleotide sequence that is at least about 65%, preferably 75%, 85%, or 95% identical to SEQ ID NO:3 or the cDNA of ATCC 98694; an isolated T139 protein which is encoded by a nucleic acid molecule having a nucleotide sequence at least about 65% preferably 75%, 85%, or 95% identical to the SCP-like domain encoding portion of SEQ ID NO:1 (e.g., about nucleotides 233 to 665 of SEQ ID NO:1), C-type lectin domain encoding portion of SEQ ID NO:1 (e.g., about nucleotides 983 to 1330 of SEQ ID NO:1), or EGF-like domain encoding portions of SEQ ID NO:1 (e.g., about nucleotides 788 to 874 and 884 to 967 of SEQ ID NO:1); and an isolated T139 protein which is encoded by a nucleic acid molecule having a nucleotide sequence which hybridizes under stringent hybridization conditions to a nucleic acid molecule having the nucleotide sequence of SEQ ID NO:3 or the non-coding strand of the cDNA of ATCC 98694.

Also within the invention is a polypeptide which is a naturally occurring allelic variant of a polypeptide that includes the amino acid sequence of SEQ ID NO:2 or SEQ ID NO:4 or an amino acid sequence encoded by the cDNA of ATCC 98694, wherein the polypeptide is encoded by a nucleic acid molecule which hybridizes to a nucleic acid molecule comprising SEQ ID NO:1 or SEQ ID NO:3 under stringent conditions.

Another embodiment of the invention features T139 nucleic acid molecules which specifically detect T139 nucleic acid molecules. For example, in one embodiment, a T139 nucleic acid molecule hybridizes under stringent conditions to a nucleic acid molecule comprising the nucleotide sequence of SEQ ID NO:1, SEQ ID NO:3, or the cDNA of ATCC 98694, or a complement thereof. In another embodiment, the T139 nucleic acid molecule is at least 300 (325, 350, 375, 400, 425, 450, 500, 550, 600, 650, 700, 800, 900, 1000, or 1290) nucleotides in length and hybridizes under stringent conditions to a nucleic acid molecule comprising the nucleotide sequence shown in SEQ ID NO:1, SEQ ID NO:3, the cDNA of ATCC 98694, or a complement thereof. In a preferred embodiment, an isolated T139 nucleic acid molecule comprises nucleotides 233 to 665 of SEQ ID NO:1, encoding the SCP-like domain of T139; nucleotides 983 to 1330 of SEQ ID NO:1, encoding the C-type lectin domain of T139; or nucleotides 788 to 874 or 884 to 967 of SEQ ID NO:1, encoding a EGF like domain of T139, or a complement thereof. In another embodiment, the invention provides an isolated nucleic acid molecule which is antisense to the coding strand of a T139 nucleic acid.

The invention features a nucleic acid molecule which is at least 45% (or 55%, 65%, 75%, 85%, 95%, or 98%) identical to the nucleotide sequence shown in SEQ ID NO:9, 11, 16, 18, 19, 21, 22, or 24, or the nucleotide sequence of the cDNA insert of the plasmid deposited with ATCC as Accession Number 98693 (the “cDNA of ATCC 98693”), or a complement thereof.

The invention features a nucleic acid molecule which includes a fragment of at least 425 (450, 500, 550, 600, 650, 700, 800, 900, 1000, or 1290) nucleotides of the nucleotide sequence shown in SEQ ID NO:9, 11, 16, 18, 19, 21, 22, or 24, or the nucleotide sequence of the cDNA of ATCC 98693, or a complement thereof.

The invention also features a nucleic acid molecule which includes a nucleotide sequence encoding a protein having an amino acid sequence that is at least 45% (or 55%, 65%, 75%, 85%, 95%, or 98%) identical to the amino acid sequence of SEQ ID NO:10, 12, 17, 20, or 23, or the amino acid sequence encoded by the cDNA of ATCC 98693. In a preferred embodiment, a T125 nucleic acid molecule has the nucleotide sequence shown SEQ ID NO:9, 11, 16, 18, 19, 21, 22, or 24, or the nucleotide sequence of the cDNA of ATCC 98693.

Also within the invention is a nucleic acid molecule which encodes a fragment of a polypeptide having the amino acid sequence of SEQ ID NO:10, 12, 17, 20, or 23, the fragment including at least 15 (25, 30, 50, 100, 150, 300, or 400) contiguous amino acids of SEQ ID NO:10, 12, 17, 20, or 23, or the polypeptide encoded by the cDNA of ATCC Accession Number 98693.

The invention includes a nucleic acid molecule which encodes a naturally occurring allelic variant of a polypeptide comprising the amino acid sequence of SEQ ID NO:10, 12, 17, 20, or 23, or an amino acid sequence encoded by the cDNA of ATCC Accession Number 98693, wherein the nucleic acid molecule hybridizes to a nucleic acid molecule comprising SEQ ID NO:9, 11, 16, 18, 19, 21, 22, or 24, under stringent conditions.

Also within the invention are: an isolated T125 protein having an amino acid sequence that is at least about 65%, preferably 75%, 85%, 95%, or 98% identical to the amino acid sequence of SEQ ID NO:12 (mature human T125) or the amino acid sequence of SEQ ID NO:10 (immature human T125), SEQ ID NO:17 (human T125a), SEQ ID NO:20 (human T125b), or SEQ ID NO:23 (human T125c); and an isolated T125 protein having an amino acid sequence that is at least about 85%, 95%, or 98% identical to the EGF1 or EGF2 domains of SEQ ID NO:10 (e.g., about amino acid residues 107 to 134 or 141 to 176 of SEQ ID NO:10).

Also within the invention are: an isolated T125 protein which is encoded by a nucleic acid molecule having a nucleotide sequence that is at least about 65%, preferably 75%, 85%, or 95% identical to SEQ ID NO:11 or the cDNA of ATCC 98693; an isolated T125 protein which is encoded by a nucleic acid molecule having a nucleotide sequence at least about 65% preferably 75%, 85%, or 95% identical the EGF-like domain encoding portions of SEQ ID NO:9 (e.g., about nucleotides 592 to 675 or 694 to 801 of SEQ ID NO:9); and an isolated T125 protein which is encoded by a nucleic acid molecule having a nucleotide sequence which hybridizes under stringent hybridization conditions to a nucleic acid molecule having the nucleotide sequence of SEQ ID NO:9, 11, 16, 18, 19, 21, 22, or 24, or the non-coding strand of the cDNA of ATCC 98693.

Also within the invention is a polypeptide which is a naturally occurring allelic variant of a polypeptide that includes the amino acid sequence of SEQ ID NO:10, 12, 17, 20, or 23, or an amino acid sequence encoded by the cDNA insert of ATCC as Accession Number 98693, wherein the polypeptide is encoded by a nucleic acid molecule which hybridizes to a nucleic acid molecule comprising SEQ ID NO:9, 11, 16, 18, 19, 21, 22, or 24, under stringent conditions.

Another embodiment of the invention features T125 nucleic acid molecules which specifically detect T125 nucleic acid molecules. For example, in one embodiment, a T125 nucleic acid molecule hybridizes under stringent conditions to a nucleic acid molecule comprising the nucleotide sequence of SEQ ID NO:9, 11, 16, 18, 19, 21, 22, or 24, or the cDNA of ATCC 98693, or a complement thereof. In another embodiment, the T125 nucleic acid molecule is at least 450 (500, 550, 600, 650, 700, 800, 900, 1000, or 1290) nucleotides in length and hybridizes under stringent conditions to a nucleic acid molecule comprising the nucleotide sequence shown in SEQ ID NO:9, SEQ ID NO:11, the cDNA of ATCC 98693, or a complement thereof. In a preferred embodiment, an isolated T125 nucleic acid molecule comprises nucleotides 592 to 675 or 694 to 801 of SEQ ID NO:9, encoding the EGF-like domains of T125, or a complement thereof. In another embodiment, the invention provides an isolated nucleic acid molecule which is antisense to the coding strand of a T125 nucleic acid.

The invention features a nucleic acid molecule which includes a fragment of at least 400 (450, 500, 550, 600, 650, 700, 800, 900, 1000, 1100, 1200, 1300, 1400, or 1420) nucleotides of the nucleotide sequence shown in SEQ ID NO:29 or a complement thereof; or a fragment of at least 200 (250, 300, 350, 400, 450, 500, 550, 600, 650, 700, 800, 900, 1000, 1110, 1200, 1300, 1400, or 1420) nucleotides of the nucleotide sequence shown in SEQ ID NO:31 or a complement thereof; or a fragment of at least 450 (500, 550, 600, 650, 700, 800, 900, 1000, 1100, 1200, 1300, 1400, or 1450) nucleotides of the nucleotide sequence shown in SEQ ID NO:33 or SEQ ID NO:35, or a complement thereof.

In a preferred embodiment, a T110 nucleic acid molecule has the nucleotide sequence shown in SEQ ID NO:29, or SEQ ID NO:31, or SEQ ID NO:33, or SEQ ID NO:35.

Also within the invention is a nucleic acid molecule which encodes a fragment of a polypeptide having the amino acid sequence of SEQ ID NO:30 or SEQ ID NO:32, or SEQ ID NO:34 or SEQ ID NO:36, the fragment including at least 70 (80, 90, 100, 120, 140, 160, 180, 200, 250, 300, 350, 400, 450, or 480) contiguous amino acids of SEQ ID NO:30 or SEQ ID NO:32; or the fragment including at least 150 (160, 170, 180, 200, 250, 300, 350, 400, 450, or 480) contiguous amino acids of SEQ ID NO:34 or SEQ ID NO:36.

The invention includes a nucleic acid molecule which encodes a naturally occurring allelic variant of a polypeptide comprising the amino acid sequence of SEQ ID NO:30 or SEQ ID NO:32, or SEQ ID NO:34 or SEQ ID NO:36, wherein the nucleic acid molecule hybridizes to a nucleic acid molecule comprising SEQ ID NO:29 or SEQ ID NO:31, or SEQ ID NO:33 or SEQ ID NO:35 under stringent conditions.

Also within the invention is an isolated T110 protein which is encoded by a nucleic acid molecule having a nucleotide sequence which hybridizes under stringent hybridization conditions to a nucleic acid molecule having the nucleotide sequence of SEQ ID NO:31, SEQ ID NO:35, or SEQ ID NO:37.

Also within the invention is a polypeptide which is a naturally occurring allelic variant of a polypeptide that includes the amino acid sequence of SEQ ID NO:30 or SEQ ID NO:32, or SEQ ID NO:34 or SEQ ID NO:36, wherein the polypeptide is encoded by a nucleic acid molecule which hybridizes to a nucleic acid molecule comprising SEQ ID NO:29 or SEQ ID NO:31, or SEQ ID NO:33 or SEQ ID NO:35 under stringent conditions;

Another embodiment of the invention features T110 nucleic acid molecules which specifically detect T110 nucleic acid molecules. For example, in one embodiment, a T110 nucleic acid molecule hybridizes under stringent conditions to a nucleic acid molecule comprising the nucleotide sequence of SEQ ID NO:29, SEQ ID NO:31, SEQ ID NO:33, or SEQ ID NO:35, or a complement thereof. In another embodiment, the T110 nucleic acid molecule is at least 440 (450, 500, 550, 600, 650, 700, 800, 900, 1000, 1100, 1200, 1300, 1400, or 1420) nucleotides in length and hybridizes under stringent conditions to a nucleic acid molecule comprising the nucleotide sequence as shown in SEQ ID NO:29 or a complement thereof; or a fragment of at least 220 (250, 300, 350, 400, 450, 500, 550, 600, 650, 700, 800, 900, 1000, 1100, 1200, 1300, 1400, or 1420) nucleotides in length and hybridizes under stringent conditions to a nucleic acid molecule comprising the nucleotide sequence as shown in SEQ ID NO:31 or a complement thereof; or a fragment of at least 450 (500, 550, 600, 650, 700, 800, 900, 1000, 1100, 1200, 1300, 1400, or 1420) nucleotides in length and hybridizes under stringent conditions to a nucleic acid molecule comprising the nucleotide sequence as shown in SEQ ID NO:33 or SEQ ID NO:35, or a complement thereof. In another embodiment, the invention provides an isolated nucleic acid molecule which is antisense to the coding strand of a T110 nucleic acid.

The invention features a nucleic acid molecule which includes a nucleotide sequence encoding a protein having an amino acid sequence that is at least 45% (or 55%, 65%, 75%, 85%, 95%, or 98%) identical to the amino acid sequence of SEQ ID NO:44, 54, 56, 63, 64, or 67.

In a preferred embodiment, a nucleic acid molecule has the nucleotide sequence shown SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:55, or SEQ ID NO:57.

Also within the invention is a nucleic acid molecule which encodes a fragment of a polypeptide having the amino acid sequence of SEQ ID NO:44, 54, 56, 63, 64, or 67, the fragment including at least 15 (25, 30, 50, 60, or 63) contiguous amino acids of SEQ ID NO:44, 54, 56, 63, 64, or 67.

The invention includes a nucleic acid molecule which encodes a naturally occurring allelic variant of a polypeptide comprising the amino acid sequence of SEQ ID NO:54 or SEQ ID NO:64, wherein the nucleic acid molecule hybridizes to a nucleic acid molecule comprising SEQ ID NO:50, SEQ ID NO:51, SEQ ID NO:52 or SEQ ID NO:53 or the complement thereof under stringent conditions.

Also within the invention are: an isolated TANGO-175 protein having an amino acid sequence that is at least about 45% (or 55%, 65%, 75%, 85%, 95%, or 98%) identical to the amino acid sequence of SEQ ID NO:64 (mature human TANGO-175) or the amino acid sequence of SEQ ID NO:54 (immature human TANGO-175).

Also within the invention are: an isolated WDNM-2 protein having an amino acid sequence that is at least about 45% (or 55%, 65%, 75%, 85%, 95%, or 98%) identical to the amino acid sequence of SEQ ID NO:46 (immature WDNM-2) or SEQ ID NO:67 (mature WDNM-2).

Also within the invention are: an isolated T175 protein which is encoded by a nucleic acid molecule having a nucleotide sequence that is at least about 65%, preferably 75%, 85%, or 95% identical to SEQ ID NO:46, SEQ ID NO:47, SEQ ID NO:48, SEQ ID NO:49, SEQ ID NO:50, SEQ ID NO:51, SEQ ID NO:52, SEQ ID NO:53; and an isolated T175 protein which is encoded by a nucleic acid molecule having a nucleotide sequence which hybridizes under stringent hybridization conditions to a nucleic acid molecule having the nucleotide sequence of SEQ ID NO:46, SEQ ID NO:47, SEQ ID NO:48, SEQ ID NO:49, SEQ ID NO:50, SEQ ID NO:51, SEQ ID NO:52, SEQ ID NO:53 or the complement thereof.

Also within the invention are: an isolated WDNM-2 protein encoded by a nucleic acid molecule having a nucleotide sequence which at least about 65%, preferably 75%, 85%, or 95% identical to SEQ ID NO:55 or 57; and an isolated WDNM-2 protein which is encoded by a nucleic acid molecule having a nucleotide sequence which hybridizes under stringent conditions to a nucleic acid molecule having the sequence of SEQ ID NO:55 or 57.

Also within the invention is a polypeptide which is a naturally occurring allelic variant of a polypeptide that includes the amino acid sequence of SEQ ID NO:54 or SEQ ID NO:64, wherein the polypeptide is encoded by a nucleic acid molecule which hybridizes to a nucleic acid molecule comprising SEQ ID NO:46, SEQ ID NO:47, SEQ ID NO:48, SEQ ID NO:49, SEQ ID NO:50, SEQ ID NO:51, SEQ ID NO:52, SEQ ID NO:53 or the complement thereof under stringent conditions.

Another embodiment of the invention features T175 nucleic acid molecules which specifically detect T175 nucleic acid molecules relative to nucleic acid molecules encoding other members of the three disulfide core superfamily.

Another aspect of the invention provides a vector, e.g., a recombinant expression vector, comprising a nucleic acid molecule of the invention. In another embodiment the invention provides a host cell containing such a vector. The invention also provides a method for producing polypeptide of the invention by culturing, in a suitable medium, a host cell of the invention containing a recombinant expression vector such that a polypeptide of the invention is produced.

Another aspect of this invention features isolated or recombinant polypeptides of the invention.

Another aspect of this invention features isolated or recombinant polypeptides of the invention. Preferred polypeptides of the invention possess at least one of the following biological activities possessed by naturally occurring human polypeptides of the invention: (1) the ability to form protein:protein interactions with proteins; (2) the ability to bind a ligand; (3) the ability to bind a receptor; (4) ability to modulate cellular proliferation; (5) ability to modulate cellular differentiation; and (6) the ability to modulate activities of tissues in which it is expressed.

The invention also features T110 proteins and polypeptides that, in addition to those listed above, possesses at least one of the following biological activities: (1) the ability to bind to an intracellular target protein; and (2) the ability to interact with a protein involved in cellular proliferation or differentiation. In one embodiment, an isolated T110 protein has an extracellular domain and lacks both a transmembrane and a cytoplasmic domain. In another embodiment, an isolated T110 protein is soluble under physiological conditions.

The invention also features T175 proteins and polypeptides that, in addition to those listed above, possesses at least one of the following biological activities: (1) the ability to inhibit a proteinase activity; (2) the ability to modulate cell-cell interactions; (3) the ability to modulate hematopoiesis (e.g., the ability to modulate proliferation of hematopoietic stem cells); (4) the ability to modulate inflammation; (5) the ability to modulate cell intravasation and/or extravasation; and (6) the ability to modulate clotting.

The polypeptides of the present invention, or biologically active portions thereof, can be operatively linked to a polypeptides not part of the invention (e.g., heterologous amino acid sequences) to form fusion proteins. The invention further features antibodies that specifically bind to polypeptides of the invention, such as monoclonal or polyclonal antibodies. In addition, the polypeptides of the invention or biologically active portions thereof can be incorporated into pharmaceutical compositions, which optionally include pharmaceutically acceptable carriers.

In another aspect, the present invention provides a method for detecting the presence of activity or expression of nucleic acids or polypeptides of the invention in a biological sample by contacting the biological sample with an agent capable of detecting an indicator of this activity such that the presence of this activity is detected in the biological sample.

In another aspect, the invention provides a method for modulating nucleic acid or polypeptide of the invention activity comprising contacting a cell with an agent that modulates (inhibits or stimulates) this activity or expression such that this activity or expression in the cell is modulated. In one embodiment, the agent is an antibody that specifically binds to polypeptide of the invention. In another embodiment, the agent modulates expression of nucleic acid or polypeptide of the invention by modulating transcription of a gene of the invention, splicing of a nucleic acid of the invention mRNA, or translation of a nucleic acid of the invention mRNA. In yet another embodiment, the agent is a nucleic acid molecule having a nucleotide sequence that is antisense to the coding strand of the nucleic acid of the invention mRNA or the gene of the invention.

In one embodiment, the methods of the present invention are used to treat a subject having a disorder characterized by aberrant expression or activity of a nucleic acid or polypeptide of the invention by administering an agent which is a nucleic acid or polypeptide of the invention modulator to the subject. In one embodiment, the nucleic acid or polypeptide of the invention modulator is a polypeptide of the invention. In another embodiment the nucleic acid or polypeptide of the invention modulator is a nucleic acid molecule of the invention. In other embodiments, the nucleic acid or polypeptide of the invention modulator is a peptide, peptidomimetic, or other small molecule. In a preferred embodiment, the disorder characterized by aberrant nucleic acid or polypeptide of the invention expression is a proliferative or differentiative disorder, particularly of the immune system. In a preferred embodiment, the disorder characterized by aberrant T110 protein or nucleic acid expression is neoplasia, inappropriate angiogenesis, or inappropriate tissue regeneration after injury. In a preferred embodiment, the disorder characterized by aberrant T175 or WDNM-2 protein or nucleic acid expression is a coagulation disorder, a proliferative disorder (e.g., cancer), an inflammatory disorder, or a hematopoietic disorder.

The present invention also provides a diagnostic assay for identifying the presence or absence of a genetic lesion or mutation characterized by at least one of: (i) aberrant modification or mutation of a gene encoding a polypeptide of the invention; (ii) mis-regulation of a gene encoding a polypeptide of the invention; and (iii) aberrant post-translational modification of a polypeptide of the invention, wherein a wild-type form of the gene encodes a protein with an activity of a polypeptide of the invention.

In another aspect, the invention provides a method for identifying a compound that binds to or modulates the activity of a polypeptide of the invention. In general, such methods entail measuring a biological activity of a polypeptide of the invention in the presence and absence of a test compound and identifying those compounds which alter the activity of the polypeptide of the invention.

The invention also features methods for identifying a compound which modulates the expression of nucleic acid or polypeptide of the invention by measuring the expression of nucleic acid or polypeptide of the invention in the presence and absence of a compound.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts the cDNA sequence (SEQ ID NO:1) and predicted amino acid sequence (SEQ ID NO:2) of human T139 (also referred to as “TANGO-139”). The open reading frame of SEQ ID NO:1 extends from nucleotide 95 to nucleotide 1432 (SEQ ID NO:3).

FIGS. 2A-2C depict alignments of portions the amino acid sequences of T139 with various consensus sequences. FIG. 2A shows the alignment of T139 amino acids 47 to 190 of SEQ ID NO:2 with the SCP-like domain consensus sequence derived from a hidden Markov model (PF00188). FIG. 2B shows the alignment of T139 amino acids 297 to 412 of SEQ ID NO:2 with the C-type lectin domain consensus sequence derived from a hidden Markov model (PF00059). FIG. 2C shows the alignment of T139 amino acids 232 to 260 (EGF1) and 264 to 291 (EGF2) of SEQ ID NO:2 with the EGF-like domain consensus sequence derived from a hidden Markov model (PF00008).

FIG. 3 is a hydropathy plot of T139. The position of cysteines (cys) are indicated by the vertical bars immediately below the plot. Relative hydrophobicity is shown above the dotted line, and relative hydrophilicity is shown below the line.

FIG. 4 depicts the cDNA sequence (SEQ ID NO:9) and predicted amino acid sequence (SEQ ID NO:10) of human T125 (also referred to as “TANGO 125”). The open reading frame of SEQ ID NO:9 extends from nucleotide 274 to nucleotide 1092 (SEQ ID NO:11).

FIG. 5 depicts an alignment of the T125 amino acids 107 to 134 (EFG1) and 141 to 176 (EGF2) of SEQ ID NO:10 with the EGF-like domain consensus sequence derived from a hidden Markov model (PF00008).

FIG. 6 is a hydropathy plot of T125. The position of cysteines (cys) are indicated by the vertical bars immediately below the plot. Relative hydrophobicity is shown above the dotted line, and relative hydrophilicity is shown below the line.

FIG. 7 depicts the cDNA sequence (SEQ ID NO:13) and predicted amino acid sequence (SEQ ID NO:15) of murine T125. The open reading frame of SEQ ID NO:13 extends from nucleotide 13 to nucleotide 837 of SEQ ID NO:13 (SEQ ID NO:14).

FIG. 8 depicts the cDNA sequence (SEQ ID NO:16) and predicted amino acid sequence (SEQ ID NO:17) of human T125a, an alternatively spliced form of human T125. The open reading frame of SEQ ID NO:16 extends from nucleotide 194 to nucleotide 442 of SEQ ID NO:16 (SEQ ID NO:18).

FIG. 9 depicts the cDNA sequence (SEQ ID NO:19) and predicted amino acid sequence (SEQ ID NO:20) of human T125b, an alternatively spliced form of T125. The open reading frame of SEQ ID NO:19 extends from nucleotide 194 to nucleotide 934 of SEQ ID NO:19 (SEQ ID NO:21)

FIG. 10 depicts the cDNA sequence (SEQ ID NO:22) and predicted amino acid sequence (SEQ ID NO:23) of T125c, an alternatively spliced form of human T125. The open reading frame of SEQ ID NO:22 extends from nucleotide 194 to nucleotide 823 of SEQ ID NO:22 (SEQ ID NO:24).

FIG. 11 depicts the cDNA sequence (SEQ ID NO:29) and predicted amino acid sequence (SEQ ID NO:30) of human T110. The open reading frame of SEQ ID NO:29 extends from nucleotide 131 to nucleotide 1441 (SEQ ID NO:31).

FIG. 12 is a hydropathy plot of human T110. The location of the predicted transmembrane (TM), and extracellular (OUT) domains are indicated as are the position of cysteines (cys; vertical bars) and potential glycosylation sites (Ngly; vertical bars). Relative hydrophobicity is shown above the dotted line, and relative hydrophilicity is shown below the dotted line.

FIG. 13 depicts the cDNA sequence (SEQ ID NO:33) and predicted amino acid sequence (SEQ ID NO:34) of mouse T110. The open reading frame of SEQ ID NO:33 extends from nucleotide 103 to nucleotide 1452 (SEQ ID NO:35).

FIG. 14 is a hydropathy plot of mouse T110. The location of the predicted transmembrane (TM), and extracellular (OUT) domains are indicated as are the position of cysteines (cys; vertical bars) and potential glycosylatin sites (Ngly; vertical bars). Relative hydrophobicity is shown above the dotted line, and relative hydrophilicity is shown below the dotted line.

FIG. 15A depicts the partial cDNA sequence of rat T110 (SEQ ID NO:37).

FIG. 15B depicts the predicted amino acid sequence (SEQ ID NO:38) of rat T110. The coding region of SEQ ID NO:38 extends from nucleotide 1 to nucleotide 507 of SEQ ID NO:37.

FIG. 16 depicts the cDNA sequence (SEQ ID NO:29) and predicted amino acid sequence (SEQ ID NO:32) of a potential alternative human T110 translation product. The open reading frame extends from nucleotide 2 to 1441 of SEQ ID NO:29 (SEQ ID NO:40).

FIG. 17 is a hydropathy plot of a potential alternative human T110 translation product. The location of the predicted transmembrane (TM), and extracellular (OUT) domains are indicated as are the position of cysteines (cys; vertical bars) and potential glycosylation sites (Ngly; vertical bars). Relative hydrophobicity is shown above the dotted line, and relative hydrophilicity is shown below the dotted line.

FIG. 18 depicts the cDNA sequence (SEQ ID NO:33) and predicted amino acid sequence (SEQ ID NO:36) of a potential alternative murine T 110 translation product. The open reading frame extends from nucleotide 1 to 1452 of SEQ ID NO:33 (SEQ ID NO:41).

FIG. 19 is a hydropathy plot of a potential alternative murine T110 translation product. The location of the predicted transmembrane (TM), and extracellular (OUT) domains are indicated as are the position of cysteines (cys; vertical bars) and potential glycosylation sites (Ngly; vertical bars). Relative hydrophobicity is shown above the dotted line, and relative hydrophilicity is shown below the dotted line.

FIG. 20 depicts the sequence alignment of D. melanogaster four jointed protein (SEQ ID NO:39) with human T110 protein (SEQ ID NO:30).

FIG. 21 is a plot showing predicted structural features of a potential alternative human T110 protein.

FIG. 22 depicts the cDNA sequence (SEQ ID NO:43) and predicted amino acid sequence (SEQ ID NO:44) of murine TANGO-175 (also referred to as “murine T175”). The open reading frame of SEQ ID NO:43 extends from nucleotide 18 to nucleotide 206 (SEQ ID NO:45).

FIGS. 23A, 23B, 23C, and 23D depict nucleic acid sequences (SEQ ID NOs:46-49) encoding human TANGO-175 (also referred to as “human T175”). The open reading frame of SEQ ID NOs:46-49 extends from nucleotide 23 to nucleotide 205 (SEQ ID NO:50-53). FIGS. 23A, 23B, 23C, and 23D also depict the amino acid sequence of human TANGO-175 (SEQ ID NO:54). The open reading frame of each of SEQ ID NOS:46-49 (SEQ ID NOs:50-53) encode the same amino acid sequence and differ only in the codon for amino acid 10.

FIG. 24 depicts the cDNA sequence (SEQ ID NO:55) and predicted amino acid sequence (SEQ ID NO:56) of murine WDNM-2. The open reading frame of SEQ ID NO:55 extends from nucleotide 37 to 264 (SEQ ID NO:57).

FIG. 25 depicts an alignment of the amino acid sequence of murine TANGO-175 (SEQ ID NO:44) with human TANGO-175 (SEQ ID NO:54). Murine and human TANGO-175 display 66.7% sequence identity in this alignment.

FIG. 26 depicts an alignment of the amino acid sequence of murine WDNM-2 (SEQ ID NO:56) with murine WDNM-1 (mWDNM-1; SEQ ID NO:58), rat WDNM-1 (rWDNM; SEQ ID NO:59), etmM031 (SEQ ID NO:60), murine TANGO-175 (mT.175orf; SEQ ID NO:44), human TANGO-175 (hT.175prot; SEQ ID NO:54), and murine anti-leukoproteinase (mALP; SEQ ID NO:61).

FIG. 27 is a hydropathy plot of murine TANGO-175. The location of the predicted transmembrane (TM), and extracellular (OUT) domains are indicated as are the position of cysteines (cys; vertical bars) and potential glycosylation sites (Ngly; vertical bars). Relative hydrophobicity is shown above the dotted line, and relative hydrophilicity is shown below the dotted line.

FIG. 28 is a hydropathy plot of human TANGO-175. The location of the predicted transmembrane (TM), and extracellular (OUT) domains are indicated as are the position of cysteines (cys; vertical bars) and potential glycosylation sites (Ngly; vertical bars). Relative hydrophobicity is shown above the dotted line, and relative hydrophilicity is shown below the dotted line.

FIG. 29 is a hydropathy plot of murine WDNM-2. The location of the predicted transmembrane (TM), and extracellular (OUT) domains are indicated as are the position of cysteines (cys; vertical bars) and potential glycosylation sites (Ngly; vertical bars). Relative hydrophobicity is shown above the dotted line, and relative hydrophilicity is shown below the dotted line.

FIG. 30 depicts the complete cDNA sequence of the clone corresponding to GenBank™ Accession No. W52431 (SEQ ID NO:62).

FIG. 31 depicts an alignment of the nucleic acid sequence of murine TANGO-175 (SEQ ID NO:43) with the EST sequence of GenBank™ Accession No. W52431 (SEQ ID NO:44).

### DETAILED DESCRIPTION OF THE INVENTION

The present invention is based, at least in part, on the discovery of a variety of cDNA molecules which encode proteins which are herein designated T139, T125, T110, T175, and WDNM-2. These proteins exhibit a variety of physiological activities, and are included in a single application for the sake of convenience. It is understood that the allowability or non-allowability of claims directed to one of these proteins has no bearing on the allowability of claims directed to the others. The characteristics of each of these proteins and the cDNAs encoding them are described separately in the ensuing sections. In addition to the full length mature and immature human proteins described in the following sections, the invention includes fragments, derivatives, and variants of these proteins, as described herein. These proteins, fragments, derivatives, and variants are collectively referred to herein as polypeptides of the invention or proteins of the invention.

TANGO-139

The present invention is based, at least in part, on the discovery of a gene encoding T139. The T139 cDNA described below (SEQ ID NO:1) has a 1338 nucleotide open reading frame (nucleotides 95-1432 of SEQ ID NO:1; SEQ ID NO:3) which encodes a 446 amino acid protein (SEQ ID NO:2). This protein includes a predicted signal sequence of about 26 amino acids (from about amino acid 1 to about amino acid 26 of SEQ ID NO:2) and a predicted mature protein of about 420 amino acids (from about amino acid 27 to amino acid 446 of SEQ ID NO:2; SEQ ID NO:4). T139 protein possesses a sperm-coating protein (SCP) domain (amino acids 47 to 190 of SEQ ID NO:2), a C-type lectin domain (amino acids 297 to 412 of SEQ ID NO:2), and two epidermal growth factor (EGF)-like domains (amino acids 232 to 260 of SEQ ID NO:2, referred to herein as the “EGF1 domain”, and amino acids 264 to 291 of SEQ ID NO:2, referred to herein as the “EGF2 domain”).

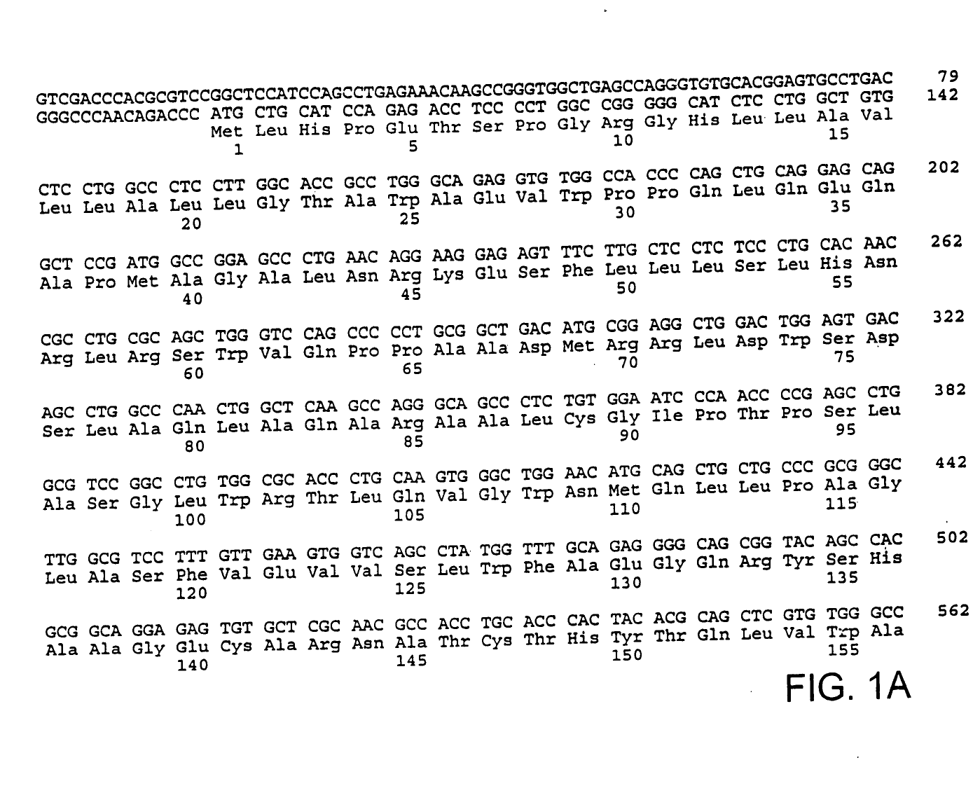

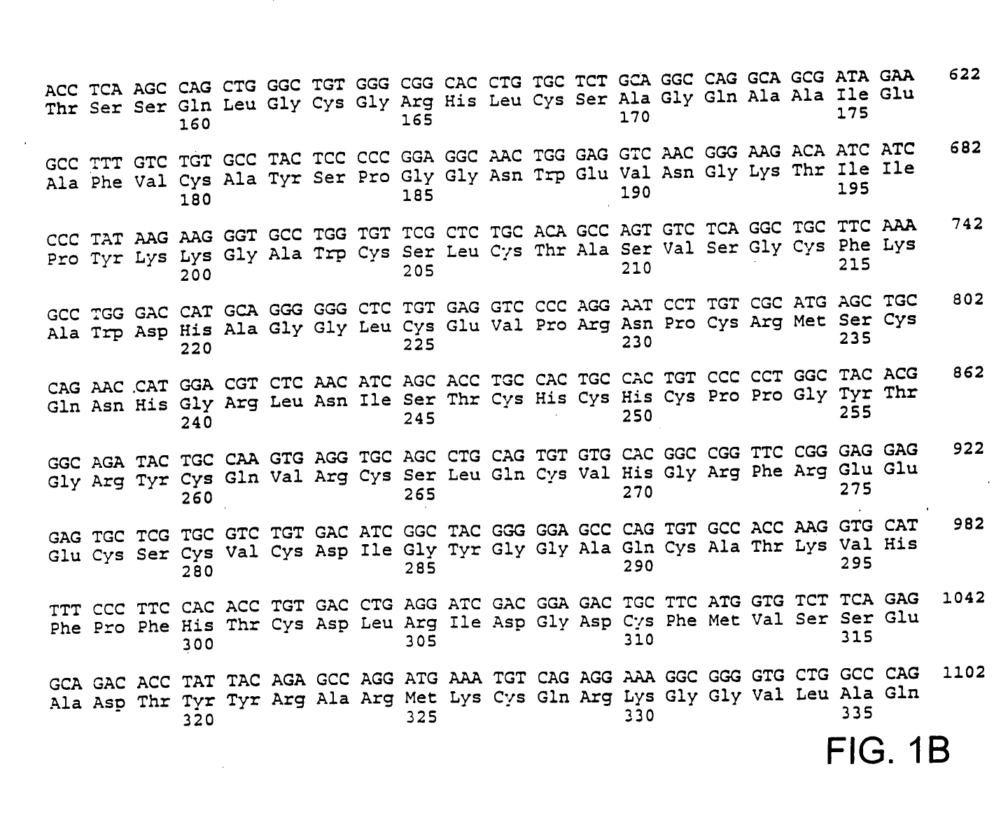

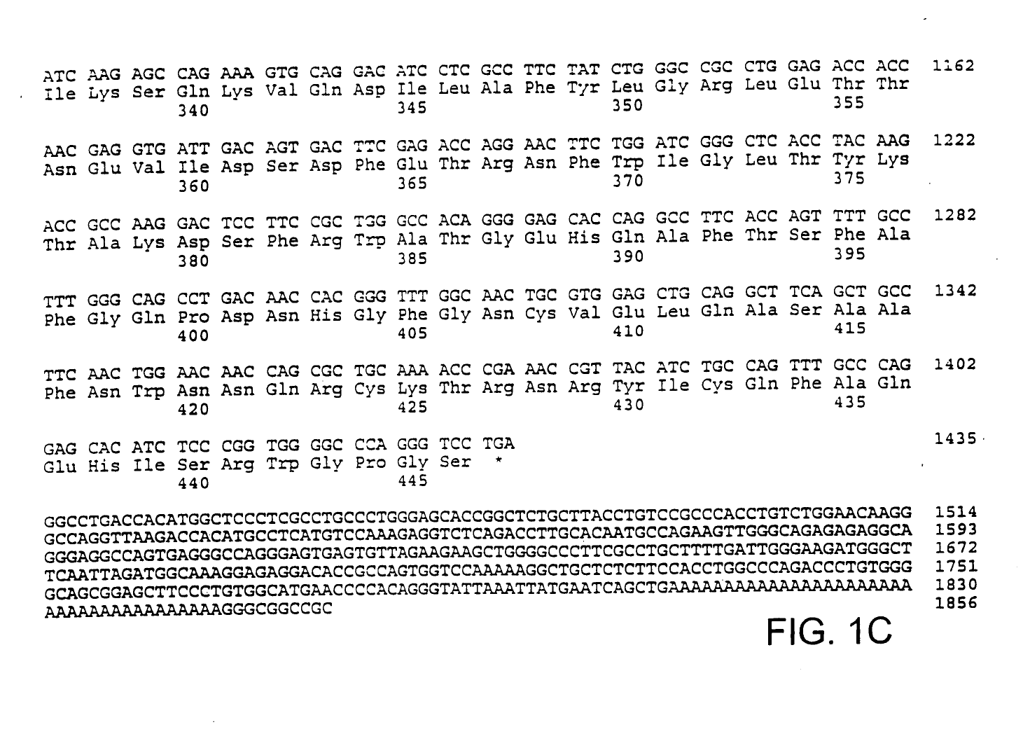

A nucleotide sequence encoding a human T139 protein is shown in FIG. 1 (SEQ ID NO:1; SEQ ID NO:3 includes the open reading frame only). A predicted amino acid sequence of T139 protein is also shown in FIG. 1 (SEQ ID NO: 2).

The T139 cDNA of FIG. 1 (SEQ ID NO:1), which is approximately 1856 nucleotides long, including untranslated regions, encodes a protein amino acid having a molecular weight of approximately 49 kDa (excluding post-translational modifications). A plasmid containing a cDNA encoding human T139 was deposited with American Type Culture Collection (ATCC), Rockville, Md. on Mar. 12, 1998, and assigned Accession Number 98694. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. 112.

Sequence analysis revealed that T139 is homologous to testis-specific protein-1 (TPX-1), a member of the SCP-like domain protein family. Comparison of the T139 SCP-like domain with the SCP-like domain consensus revealed that the T139 SCP-like domain is 28% identical (45/162 amino acids) and 50% similar (81/162 amino acids) to the consensus.

Alignment of the C-type lectin domain of human T139 protein with the C-type lectin domain consensus sequence revealed that the domains are 27% identical (28/103 amino acids) and 63% similar (65/103 amino acids). C-type lectin domains appear to function as calcium-dependent carbohydrate-recognition domains and contain four conserved cysteines. The first and fourth cysteines and the second and third cysteines in the consensus participate in disulfide bonding with each other. One example of a protein having a C-type lectin domain is the REG protein, a 166 amino acid polypeptide shown to stimulate beta-cell regeneration in a adult mouse pancreas. For a review on the REG protein, see Baeza et al. (1996) Diab. Metab. 22:229-234.

Alignment of the EGF-like domains of human T139 protein with the EGF-like domain consensus sequence revealed that the EGF1 domain is 38% identical (13/34 amino acids) and 71% similar (24/34 amino acids). In general, EGF-like domains are found in the extracellular portion of membrane-bound proteins or in secreted proteins. EGF-like domains typically include six cysteine residues involved in disulfide bond formation with two conserved glycines between the fifth and sixth cysteine. The secondary structure of EGF-like domains appears to be a two-stranded B-sheet followed by a loop to a C-terminal short two-stranded sheet.

Tango 139 is expressed at high levels in the kidney and at low levels in the testis as an about 2.0 kb transcript. Additional T139 transcripts of about 2.4 kb and 3.5 kb were also present in these two tissues. No T139 expression was observed in the heart, brain, placenta, lung, liver, skeletal muscle, pancreas, spleen, thymus, ovaries, small intestine, colon, and peripheral blood leukocytes.

Human T139 is one member of a family of molecules (the “T139 family”) having certain conserved structural and functional features. The term “family” when referring to the protein and nucleic acid molecules of the invention is intended to mean two or more proteins or nucleic acid molecules having a common structural domain and having sufficient amino acid or nucleotide sequence identity as defined herein. Such family members can be naturally occurring and can be from either the same or different species. For example, a family can contain a first protein of human origin and a homologue of that protein of murine origin, as well as a second, distinct protein of human origin and a murine homologue of that protein. Members of a family may also have common functional characteristics as described herein.

In one embodiment, a T139 protein includes a SCP-like, C-type lectin, or EGF-like domain having at least about 65%, preferably at least about 75%, and more preferably about 85%, 95%, or 98% amino acid sequence identity, to the SCP-like, C-type lectin, or EGF-like (that is, EGF1 or EGF2) domains of SEQ ID NO:2.

Preferred T139 polypeptides of the present invention have an amino acid sequence sufficiently identical to the SCP-like, C-type lectin, or EGF-like (that is, EGF1 or EGF2) domains of SEQ ID NO:2. As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences have a common structural domain and/or common functional activity. For example, amino acid or nucleotide sequences which contain a common structural domain having about 65% identity, preferably 75% identity, more preferably 85%, 95%, or 98% identity are defined herein as sufficiently identical.

As used interchangeably herein an T139 “activity”, “biological activity of T139” or “functional activity of T139”, refers to an activity exerted by a T139 protein, polypeptide or nucleic acid molecule encoding a T139 polypeptide on a T139 responsive cell as determined in vivo, or in vitro, according to standard techniques. A T139 activity can be a direct activity, such as an association with or an enzymatic activity on a second protein or an indirect activity, such as a cellular signaling activity mediated by interaction of the T139 protein with a second protein (e.g., a T139 receptor). In a preferred embodiment, a T139 activity includes at least one or more of the following activities: (i) interaction with other proteins; (ii) interaction with a T139 receptor;. Note that the above definition and explanation of “activity”, “biological activity”, and “functional activity” also applies to other nucleic acids and polypeptides of the invention (e.g., T125, T110, and T175).

Accordingly, another embodiment of the invention features isolated T139 proteins and polypeptides having a T139 activity.

Yet another embodiment of the invention features T139 molecules which contain a signal sequence. Generally, a signal sequence (or signal peptide) is a peptide containing about 20 (e.g, 15-30, or 20-30)] amino acids which occurs at the extreme N-terminal end of secretory and integral membrane proteins and which contains large numbers of hydrophobic amino acid residues and serves to direct a protein containing such a sequence to a lipid bilayer.

TANGO-125

The present invention is based, at least in part, on the discovery of a gene encoding T125. The T125 cDNA described below (SEQ ID NO:9) has a 819 nucleotide open reading frame (nucleotides 274-1092 of SEQ ID NO:9; SEQ ID NO:11) which encodes a 273 amino acid protein (SEQ ID NO:10). This protein includes a predicted signal sequence of about 22 amino acids (from amino acid 1 to about amino acid 22 of SEQ ID NO:10) and a predicted mature protein of about 252 amino acids (from about amino acid 23 to amino acid 274 of SEQ ID NO:10; SEQ ID NO:12). T125 protein possesses two epidermal growth factor (EGF)-like domains: amino acids 107 to 134 of SEQ ID NO:10, referred to herein as the “EGF1 domain”, and amino acids 141 to 176 of SEQ ID NO:10, referred to herein as the “EGF2 domain”. T125 is predicted to have no transmembrane domains and appears to be a secreted protein.

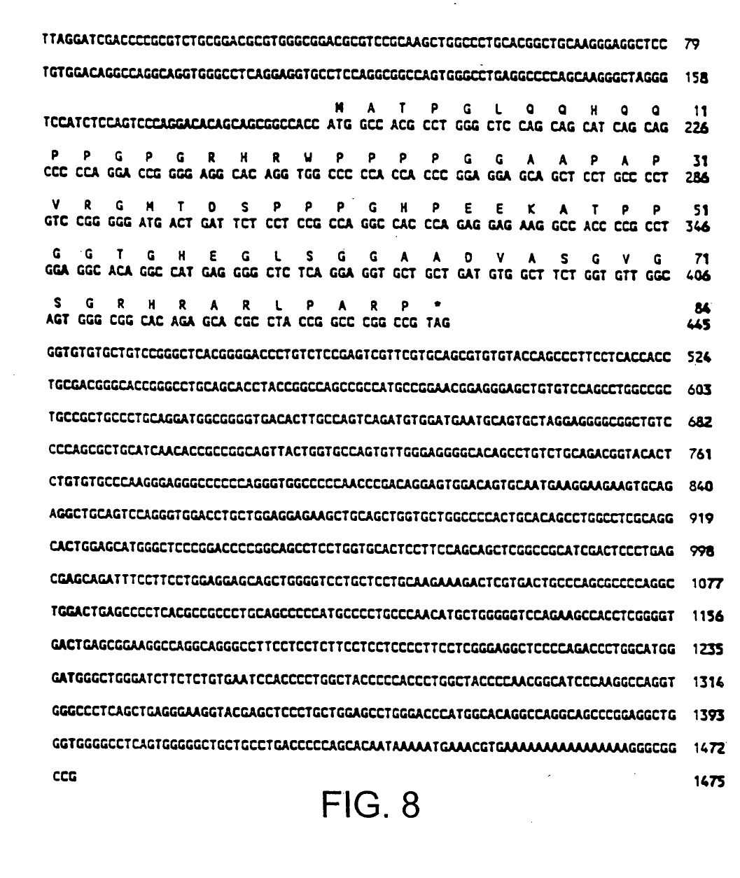

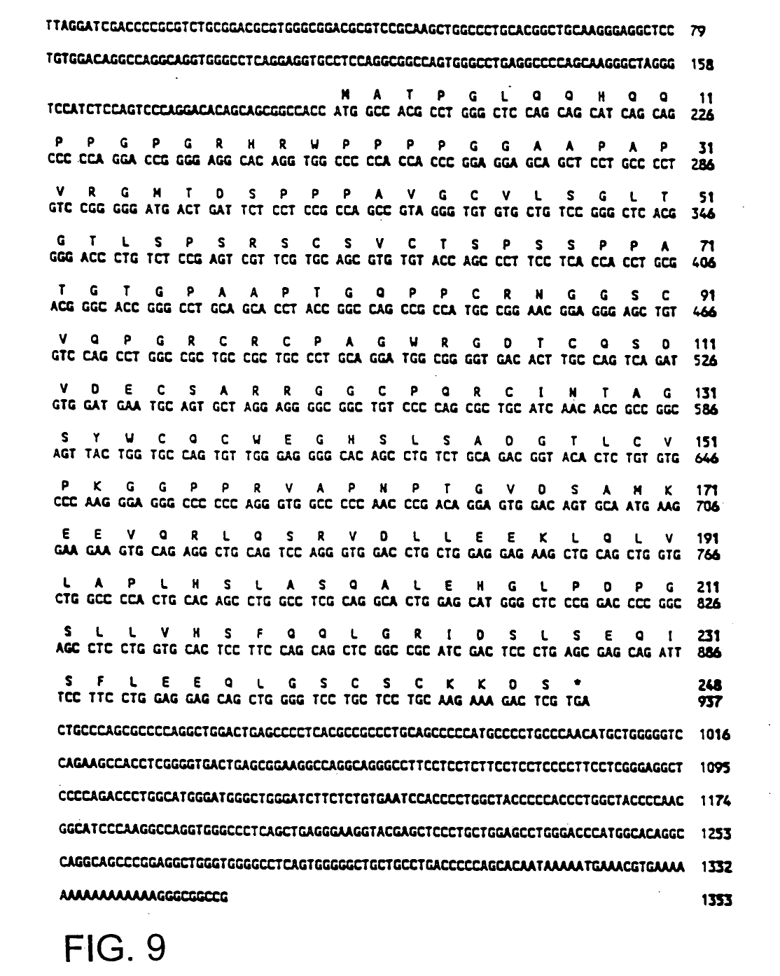

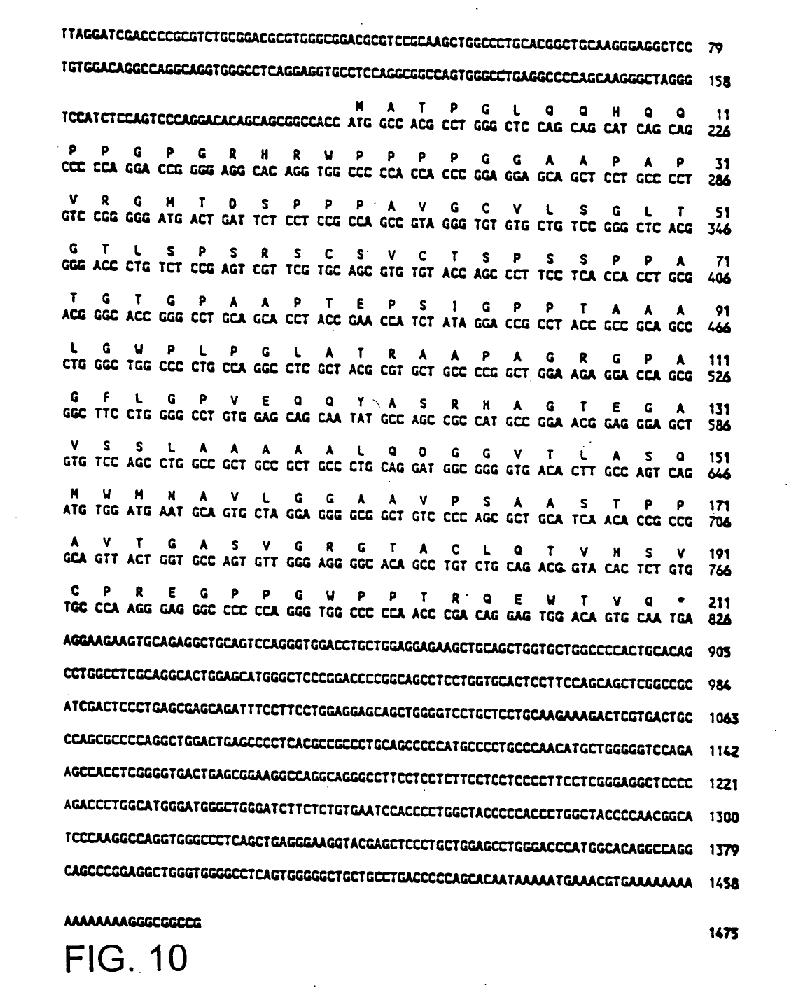

In addition, there are three additional alternatively spliced forms of human T125: T125a, T125b, and T125c. FIG. 8 depicts the cDNA sequence (SEQ ID NO:16) and predicted amino acid sequence (SEQ ID NO:17) of human T 125a. The open reading frame of SEQ ID NO:16 extends from nucleotide 194 to nucleotide 442 of SEQ ID NO:16 (SEQ ID NO:18). FIG. 9 depicts the cDNA sequence (SEQ ID NO:19) and predicted amino acid sequence (SEQ ID NO:20) of human T125b. The open reading frame of SEQ ID NO:19 extends from nucleotide 194 to nucleotide 934 of SEQ ID NO:19 (SEQ ID NO:21). Figure 10 depicts the cDNA sequence (SEQ ID NO:22) and predicted amino acid sequence (SEQ ID NO:23) of T125c. The open reading frame of SEQ ID NO:22 extends from nucleotide 194 to nucleotide 823 of SEQ ID NO:22 (SEQ ID NO:24).

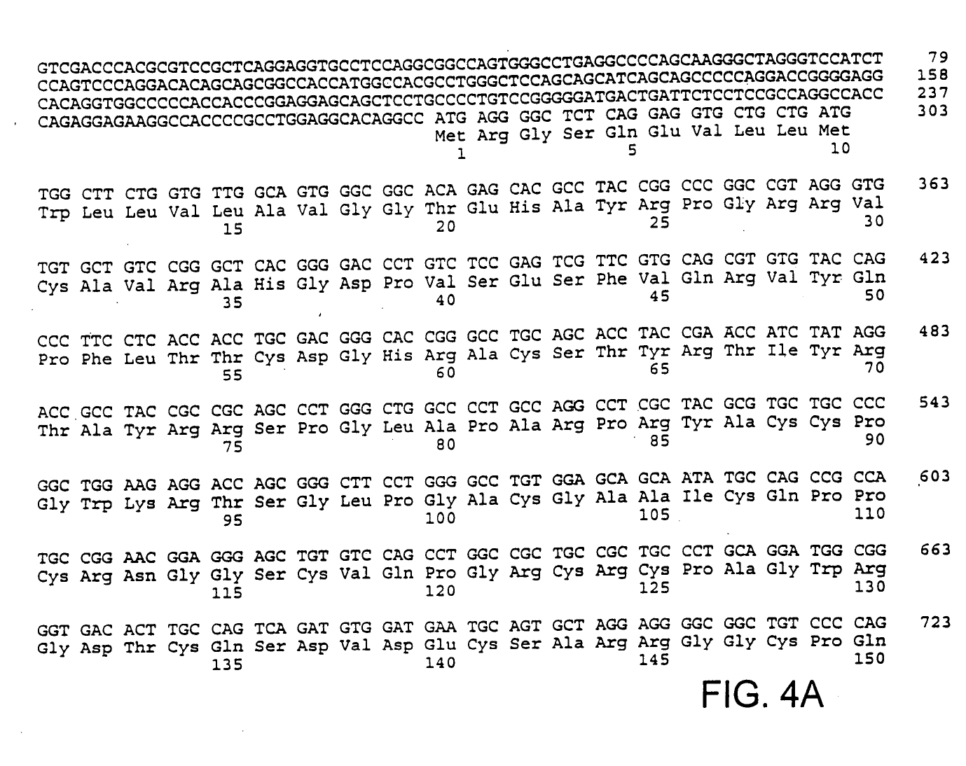

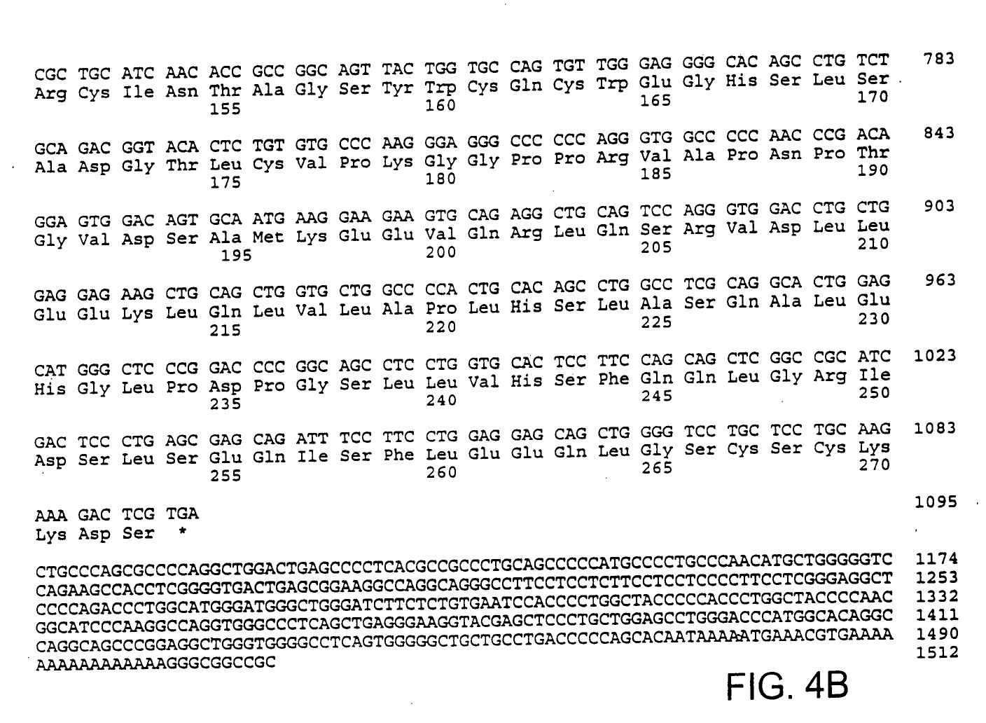

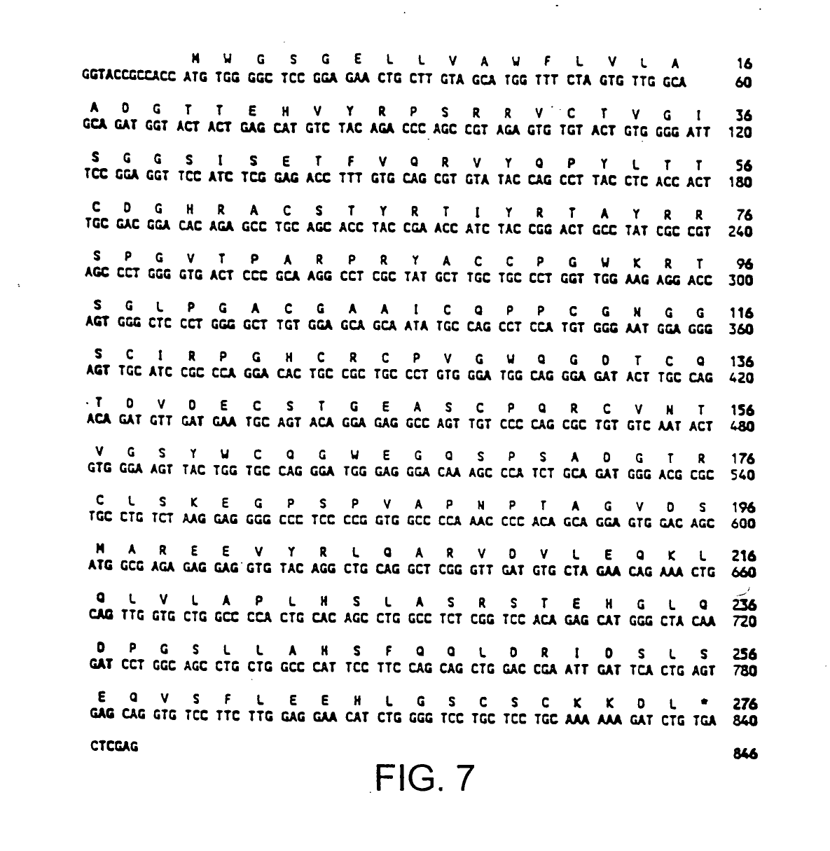

A nucleotide sequence encoding a human T125 protein is shown in FIG. 4 (SEQ ID NO:9; SEQ ID NO:11 includes the open reading frame only). A predicted amino acid sequence of T125 protein is also shown in FIG. 4 (SEQ ID NO:10). A cDNA sequence encoding a murine T125 protein is shown in FIG. 7 (SEQ ID NO:13). The open reading frame only of this cDNA (nucleotides 13-837 of SEQ ID NO:13; SEQ ID NO:14) encodes a 275 amino acid protein (SEQ ID NO:15) that is also shown in FIG. 7.

Unless otherwise specified, “T125” (or “TANGO 125”) is used to refer to all forms of T125 (T125, T125a, T125b, and T125c).

The T125 cDNA of FIG. 4 (SEQ ID NO:9), which is approximately 1512 nucleotides long including untranslated regions, encodes a protein amino acid having a molecular weight of approximately 30 kDa (excluding post-translational modifications). A plasmid containing a cDNA encoding human T125 (with the plasmid name of pDH169) was deposited with American Type Culture Collection (ATCC), Rockville, Md. on Mar. 12, 1998 and assigned Accession Number 98693. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. 112.

Sequence analysis revealed that T125 is homologous to GenBank entry gi-1841553, a protein having two EGF-like domains.

Alignment of the EGF-like domains of human T125 protein with an EGF-like domain consensus sequence derived from a hidden Markov model revealed that the EGF1 domain is 44% identical (15/34 amino acids) and 65% similar (22/34 amino acids) and that the EGF2 domain is 35% identical (12/34 amino acids) and 71% similar (24/34 amino acids). In general, EGF-like domains are found in the extracellular portion of membrane-bound proteins or in secreted proteins. EGF-like domains typically include six cysteine residues involved in disulfide bond formation with two conserved glycines between the fifth and sixth cysteine. The secondary structure of EGF-like domains appears to be a two-stranded B-sheet followed by a loop to a C-terminal short two-stranded sheet.

T125 is expressed as a series of transcripts between 1.3 and 3 kb, which are expressed at various levels in the spleen, thymus, prostate, testes, ovary, small intestine, colon, heart, brain, placenta, lung, liver, skeletal muscle, kidney, and pancreas, the highest level of expression being observed in the placenta. T125 mRNA was not detected in peripheral blood leukocytes.

Human T125 is one member of a family of molecules (the “T125 family”) having certain conserved structural and functional features. The term “family” is defined and described above.

In one embodiment, a T125 protein includes an EGF-like domain having at least about 65%, preferably at least about 75%, and more preferably about 85%, 95%, or 98% amino acid sequence identity to the EGF-like (that is, EGF1 or EGF2) domains of SEQ ID NO:10.

Preferred T125 polypeptides of the present invention have an amino acid sequence sufficiently identical to the amino acid sequences of the EGF-like (that is, EGF1 or EGF2) domains of SEQ ID NO:10. The term “sufficiently identical” is defined and described above.

“Activity”, “biological activity”, and “functional activity” are all defined and described above, and apply in all respects to T125.

Accordingly, another embodiment of the invention features isolated T125 proteins and polypeptides having a T125 activity.

Yet another embodiment of the invention features T125 molecules which contain a signal sequence. “Signal sequence” is defined and described above.

TANGO-110

The present invention is based, at least in part, on the discovery of a gene encoding T110. T110 protein is related to four-jointed (fj) protein of Drosophila melanogaster. T110 is predicted to be a member of the type-II membrane protein superfamily. Such proteins usually employ a transmembrane domain as the internal signal sequence. The amino terminal end of such proteins is normally intracellular, and the carboxy terminal end is normally extracellular. However, some type II membrane proteins are secreted from the cell while others are initially expressed on the surface of the cell and are subsequently processed to release a soluble fragment.

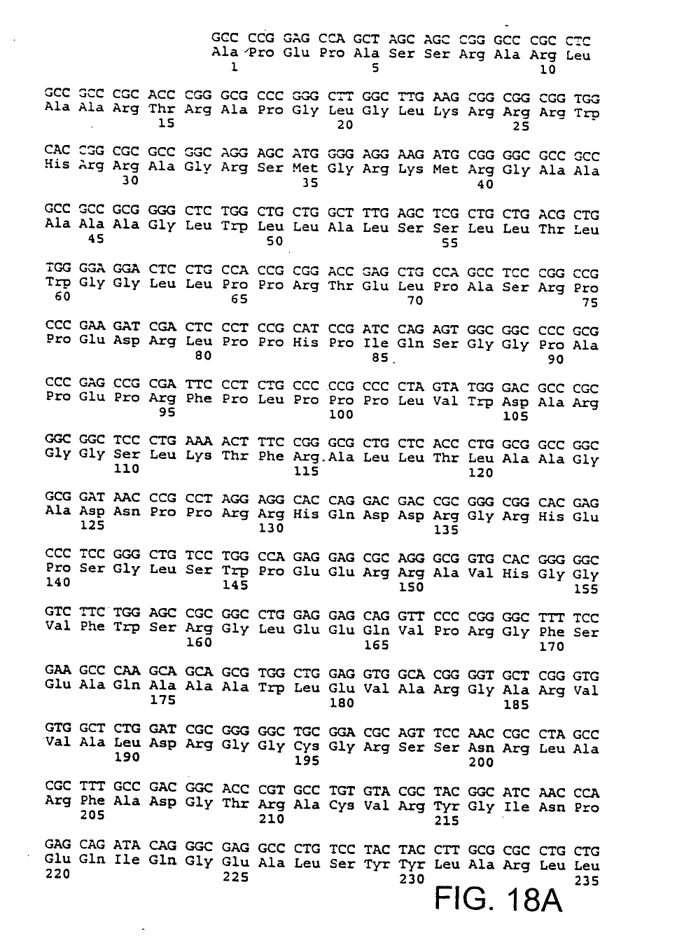

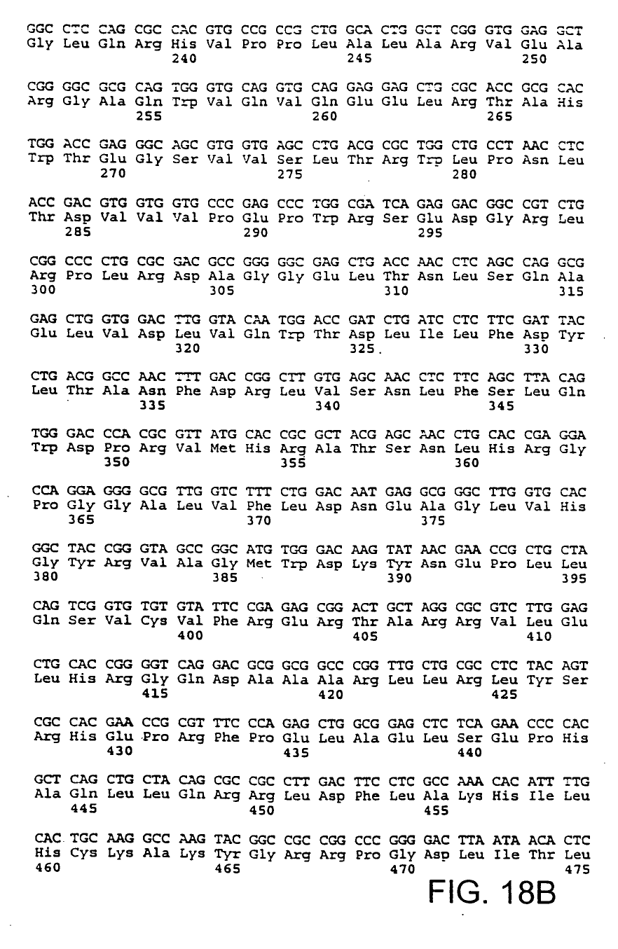

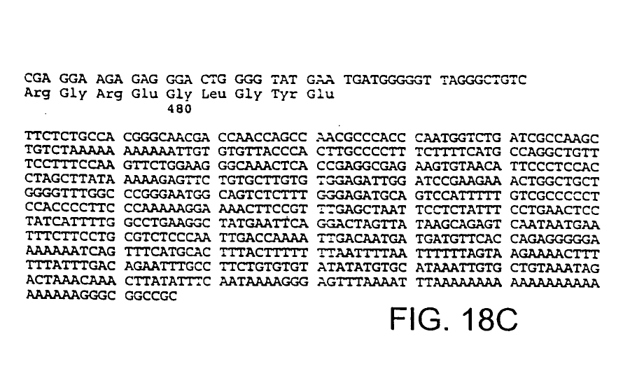

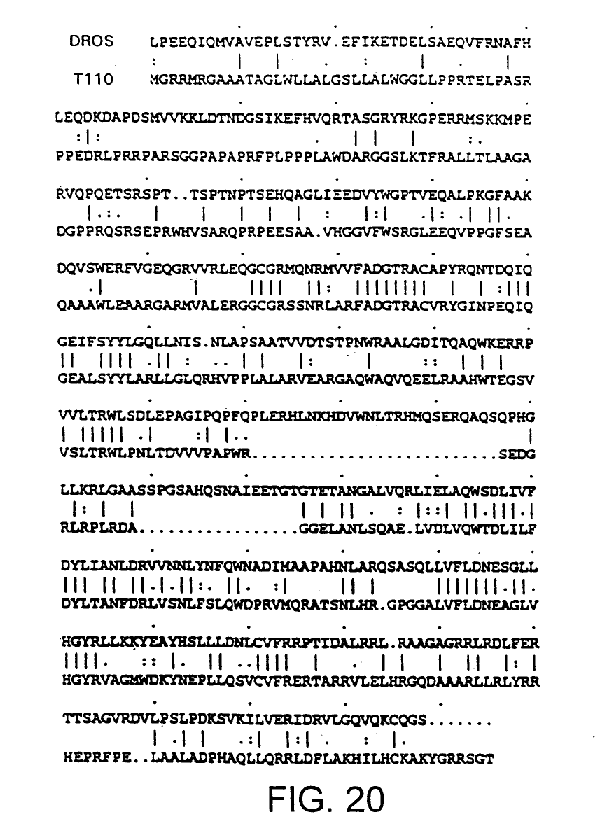

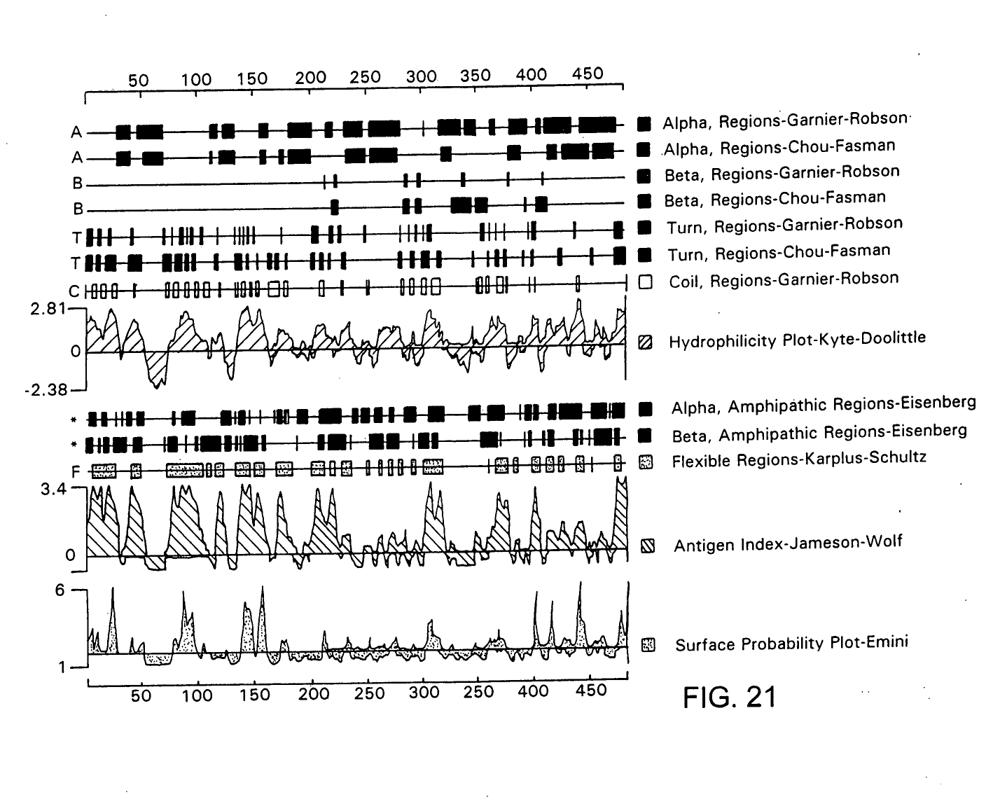

The human T110 cDNA described below (SEQ ID NO:29) has a 1311 nucleotide open reading frame (nucleotides 131 to 1441 of SEQ ID NO:29; SEQ ID NO:31) which encodes a 437 amino acid protein (SEQ ID NO:30). FIG. 18 depicts a potential alternative translation product (SEQ ID NO:32) for the above-described human T110 cDNA. It is possible that this alternative translation product is not full length. Those skilled in the art can isolate full-length clones having additional 5′ coding sequence using the methods described below.

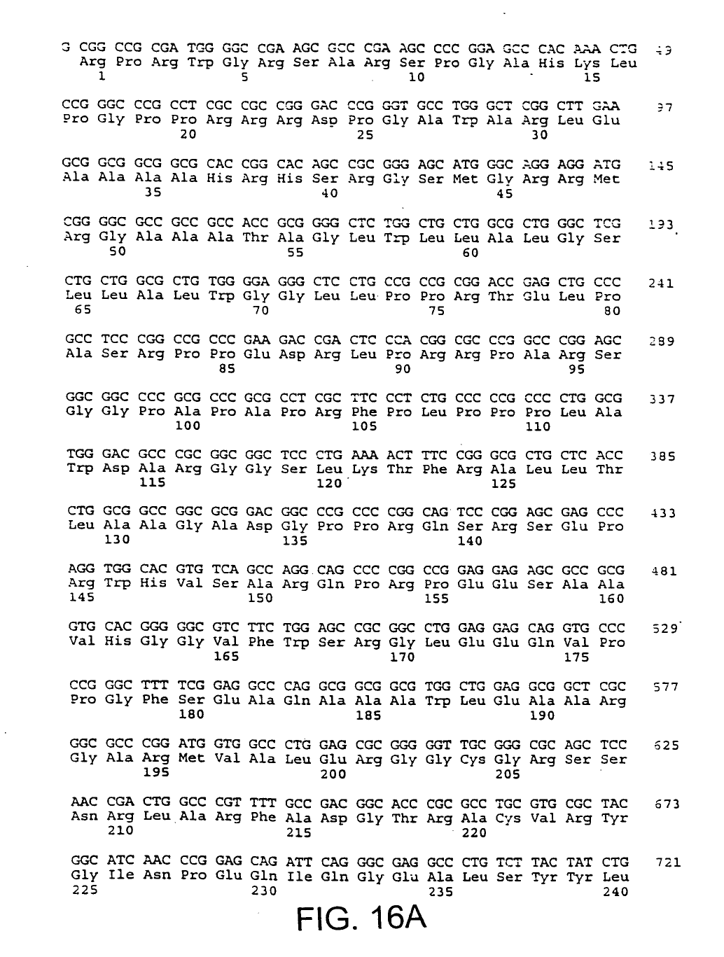

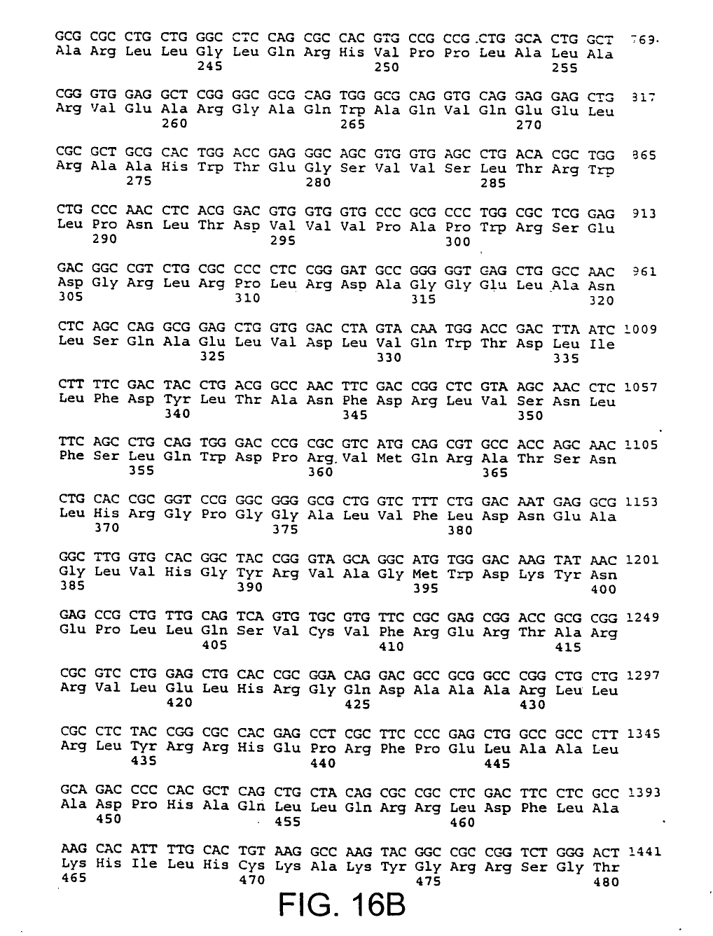

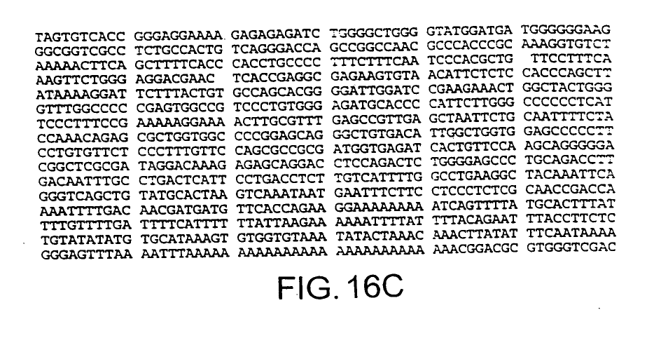

The mouse T110 cDNA described below (SEQ ID NO:33) has a 1350 nucleotide open reading frame (nucleotides 103 to 1452 of SEQ ID NO:33; SEQ ID NO:35) which encodes a 450 amino acid protein (SEQ ID NO:34). FIG. 16 depicts a potential alternative translation product (SEQ ID NO:36) for the above-described murine T110 cDNA. It is possible that this alternative translation product is not full length. Those skilled in the art can isolate full-length clones having additional 5′ coding sequence using the methods described below.

A partial rat T110 cDNA is also described below (SEQ ID NO:37). It has a 507 nucleotide open reading frame (nucleotides 1 to 507 of SEQ ID NO:37) which encodes a 169 amino acid peptide (SEQ ID NO:38). Those skilled in the art can isolate full-length clones having additional 5′ sequence using the methods described below.

A plasmid containing DNA encoding murine T110 and a plasmid containing DNA encoding human T110 were deposited with the American Type Culture Collection (ATCC), 10801 University Boulevard, Manassas, Va., 20110-2209, on Jun. 22, 1998, and have been assigned ATCC Accession Nos. 98801 and 98802, respectively. The deposits were made according to the terms of the Budapest Treaty on the International Recognition of the Deposit of Microorganisms for the Purpose of Patent Procedure. The plasmid containing human DNA was deposited in E. coli (strain designation Epfthb 110d), which contains a human T110 DNA in the plasmid vector pZL1. The plasmid containing murine DNA was also deposited in E. coli (strain designation Epftmb 110 g), which contains a murine Ti 10 DNA in the plasmid vector pZL1. The deposits were made merely as a convenience for those of skill in the art and are not an admission that deposits are required under 35 U.S.C. 0.112.

The invention includes a nucleic acid molecule that contains the nucleotide sequence of the cDNA having ATCC Accession No. 98801, or ATCC Accession No. 98802, the coding sequence of that cDNA (i.e., the cDNA having ATCC Accession No. 98801, or ATCC Accession No. 98802), or complements thereof. Similarly, the invention includes a nucleic acid molecule that contains the nucleotide sequence of the cDNA having ATCC Accession No. 98801, or ATCC Accession No. 98802, the coding sequence of that cDNA (i.e., the cDNA having ATCC Accession No. 98801, or ATCC Accession No. 98802), or complements thereof.

The invention includes polypeptides encoded by the coding sequence of the nucleic acid molecules described above, i.e., sequence contained within the nucleic acid molecules deposited with the ATCC and assigned ATCC Accession Nos. 98801 and 98802, and biologically active fragments thereof. Moreover, those of ordinary skill in the art will recognize that many, if not all, of the methods described herein can be practiced with the nucleic acid molecules (or complements or fragments thereof) deposited with the ATCC, as described above, and/or the polypeptides (or fragments thereof) encoded by those molecules, just as they can be practiced as described herein by reference to a given SEQ ID NO.

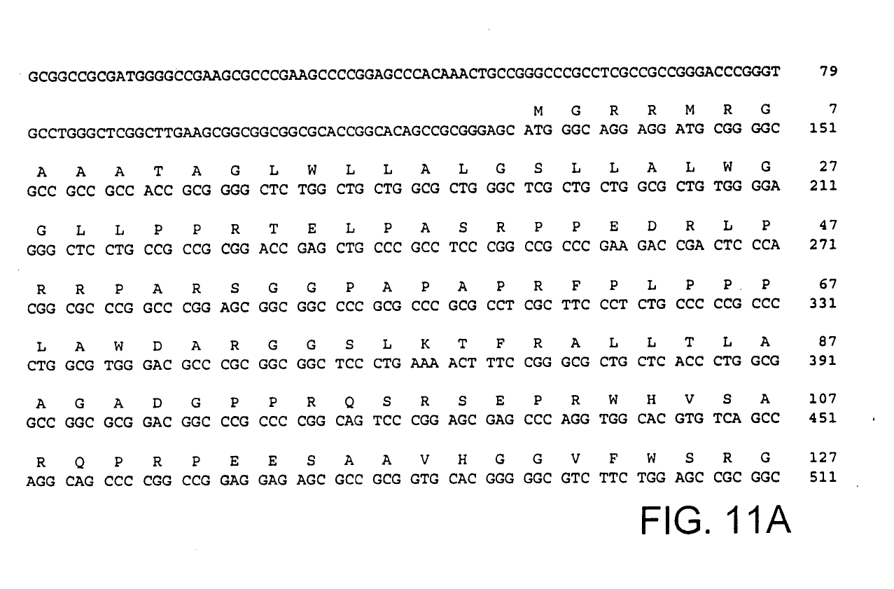

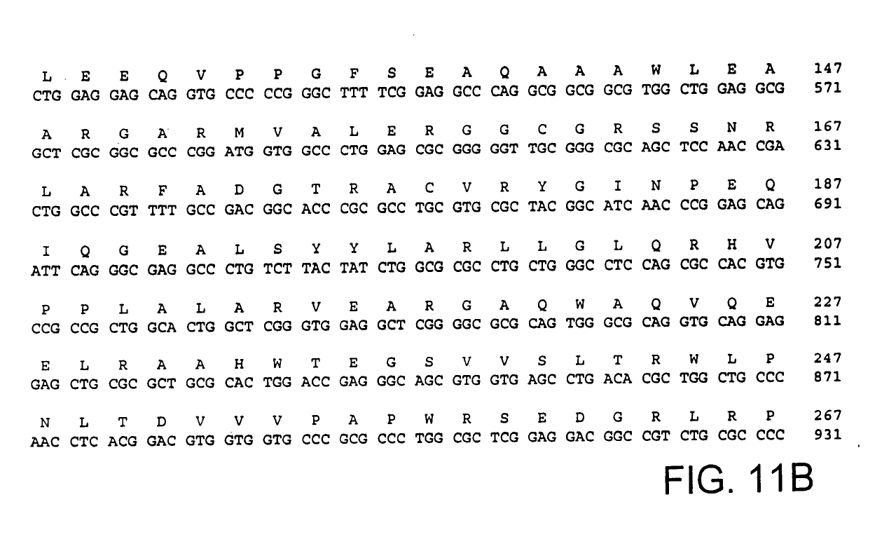

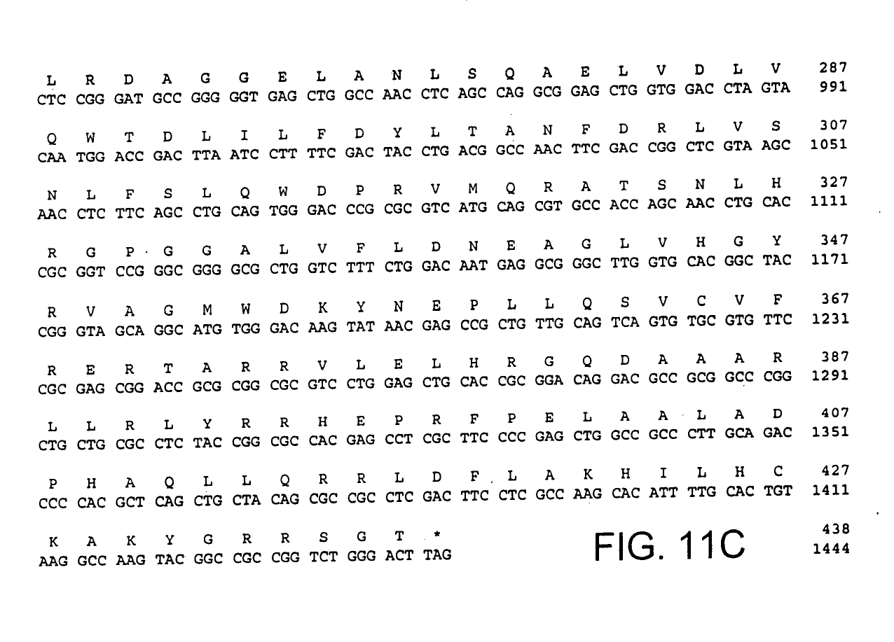

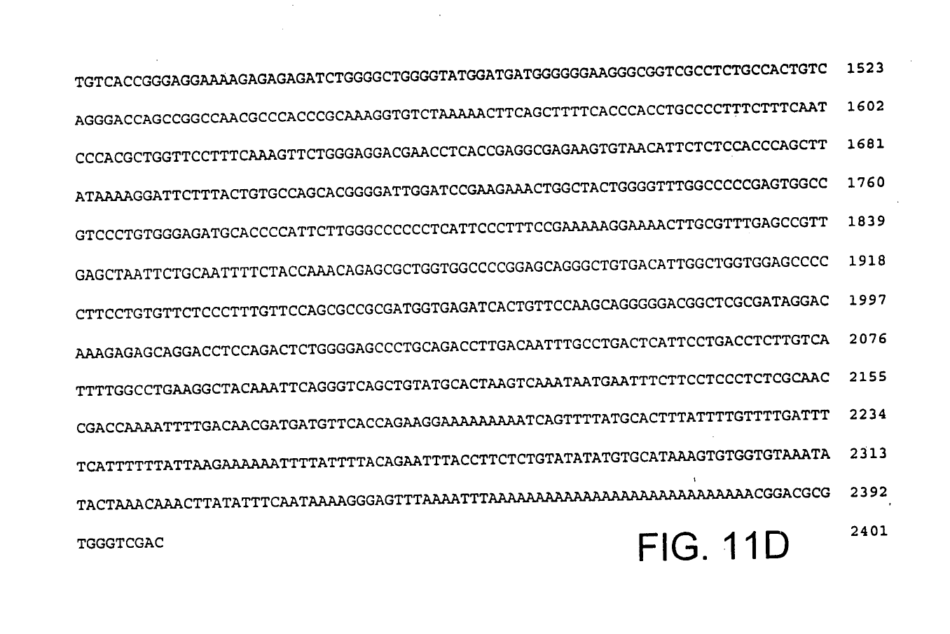

The present invention is based on the discovery of a cDNA molecule encoding human T110, a member of the type-II membrane protein superfamily. A nucleotide sequence encoding a human T 10 protein is shown in FIG. 11 (SEQ ID NO:29; SEQ ID NO:31 includes the open reading frame only). A predicted amino acid sequence of T110 protein is also shown in FIG. 11 (SEQ ID NO:30). This protein includes a predicted signal peptide of about 28 amino acids (from amino acid 1 to about amino acid 28 of SEQ ID NO:30). The predicted mature protein extends from about amino acid 29 to amino acid 437 of SEQ ID NO:30 (SEQ ID NO:42).

The human T110 cDNA of FIG. 11 (SEQ ID NO:29), which is approximately 2401 nucleotides long including untranslated regions, encodes a protein amino acid having a molecular weight of approximately 48 kDa (excluding post-translational modifications).

Human T110 protein and D. melanogaster four-jointed (fj) protein share many primary features. They are proteins of similar size and both contain a single predicted hydrophobic region near the N-terminus that may be a transmembrane domain rather than a signal sequence. Thus, the hydrophobic region from amino acids 1-28 (or 7-30) might be a transmembrane domain that acts as an internal signal sequence. Each protein contains two pairs of conserved cysteine residues, one pair near the center of the molecule (cys₁₆₁, and cys₁₇₈), the other pair near the C-terminus of the molecule (cys₃₆₅ and cys₄₂₇). Regions of highest identity between the two proteins surround the two pairs of cysteines in the extracellular domains. Each protein also contains putative N-glycosylation sites, two of which are in approximately the same position, i.e., between the two pairs of cysteines (amino acid residuess 248 to 251 and amino acid residues 277 to 280). A sequence alignment of human T110 protein and D. melanogaster fj protein is depicted in FIG. 16. In this alignment, human T110 protein and D. melanogaster fj protein display about 30% identity and about 36% similarity.

An approximately 2.4 kb human T110 mRNA transcript is expressed at the highest level in brain, heart, placenta, and pancreas. Low levels of this transcript have been observed in liver, skeletal muscle, and kidney. No detectable message is seen in lung. Embryonic expression is seen in week 8-9 fetus and week 20 liver and spleen mixed tissues. Embryonic expression is also observed in neuronal tissue.

Human T110 is one member of a family of molecules (the “T110 family”) having certain conserved structural and functional features. The present invention provides detailed description of various members of the “T110 family”, e.g., human T110, mouse T110, and rat T110. The term “family” is defined and described above.

Preferred T110 polypeptides of the present invention have an amino acid sequence sufficiently identical to the consensus amino acid sequence of human T110 protein. The term “sufficiently identical” is defined and described above.

“Activity”, “biological activity”, and “functional activity” are all defined and described above, and apply in all respects to T110. In a preferred embodiment, a T110 activity includes at least one or more of the following activities: (i) the ability to interact with proteins in the T110 signalling pathway (ii) the ability to interact with a T110 ligand or receptor (iii) the ability to interact with an intracellular target protein; and (iv) the ability to interact with proteins involved in cellular proliferation or differentiation.

Accordingly, another embodiment of the invention features isolated T110 proteins and polypeptides having a T110 activity.

TANGO-175 and WDNM-2

The mouse TANGO-175 cDNA described below (SEQ ID NO:43) has a 189 nucleotide open reading frame (nucleotides 18-206 of SEQ ID NO:43; SEQ ID NO:45) which encodes a 63 amino acid protein (SEQ ID NO:44). This protein includes a predicted signal sequence of about 24 amino acids (from amino acid 1 to about amino acid 24 of SEQ ID NO:44) and a predicted mature protein of about 39 amino acids (from about amino acid 25 to amino acid 63 of SEQ ID NO:44; SEQ ID NO:63). Murine TANGO-175 protein possesses six cysteine residues, C₁-C₆, which occur at amino acid 35, 39, 45, 51, 56 and 60 of SEQ ID NO:44, respectively. These cysteine residues are expected to form interdomain disulfide bonds which stabilize the TANGO-175 protein. Cysteines C1-C5, C2-C4 and C3-C6 are expected to form disulfide bonds. Murine TANGO-175 protein has some sequence similarity to murine WDNM-1 protein (SEQ ID NO:58; Dear & Kefford (1991) Biochem & Biophy. Res. Comm. 176:247; EMBL database accession no. X13309); trout anti-leukoproteinase (Genbank accession no. U03890), rat WDNM-1 (SEQ ID NO:59; Genbank accession no. P14730), human anti-leukoproteinasse (Goselink et al.(1996) J. Exp Med 184:1305-12), and murine anti-leukoproteinase (SLP1) (SEQ ID NO:61; Jin et al. (1997) Cell 88:417-26; Genbank accession no. P97430).

Four nucleotide sequences encoding human TANGO-175 are described below (SEQ ID NO:46, 47, 48, and 49). Each of these sequences has a 183 nucleotide open reading frame (nucleotides 23-205 of SEQ ID NO:46, 47, 48, and 49; SEQ ID NO:50, 51, 52, and 53) which encodes a 61 amino acid protein (SEQ ID NO:54). The four sequences differ only at nucleotide 52 (the third nucleotide in the codon encoding Valine at residue 10). The human TANGO-175 protein includes a predicted signal sequence of about 24 amino acids (from amino acid 1 to about amino acid 24 of SEQ ID NO:54) and a predicted mature protein of about 37 amino acids (from about amino acid 25 to amino acid 61 of SEQ ID NO:54; SEQ ID NO:64).

Human TANGO-175 protein possesses six cysteine residues, cysteines C1-C6, which occur at amino acids 33, 37, 43, 49, 54 and 58 of SEQ ID NO:54, respectively. These cysteine residues are expected to form interdomain disulfide bonds which stabilize the human TANGO-175 protein. Cysteines C1-C5, C2-C4 and C3-C6 are expected to form disulfide bonds. Like murine TANGO-175, human TANGO-175 protein has some sequence similarity to murine WDNM-1 protein (SEQ ID NO:58; Dear & Kefford (1991) Biochem & Biophy. Res. Comm. 176:247; EMBL database accession no. X13309), trout anti-leukoproteinase (Genbank accession no. U03890), rat WDNM-1 (SEQ ID NO:59; Genbank accession no. P14730), human antileukoproteinasse (Goselink et al.(1996) J. Exp Med 184:1305-12), and murine anti-leukoproteinase (SLP1) (SEQ ID NO:61; Jin et al. (1997) Cell 88:417-26; Genbank accession no. P97430).

Both murine and human TANGO-175 have six cysteines that are spaced identically to cysteines 2, 3, 4, 5, 7, and 8 of murine WDNM-1, a four-disulfide core protein. However, murine and human TANGO-175 lack equivalents of cysteines 1 and 6 present in murine WDNM-1. Thus, rather than following the 1-6,2-7, 3-5, and 4-8 disulfide bonding pattern found in the four-disulfide core proteins, TANGO-175 likely follows a 1-5,2-4, and 3-6 disulfide bonding pattern (corresponding to the 2-7,3-5, and 4-8 disulfide bonds of WDNM-1).

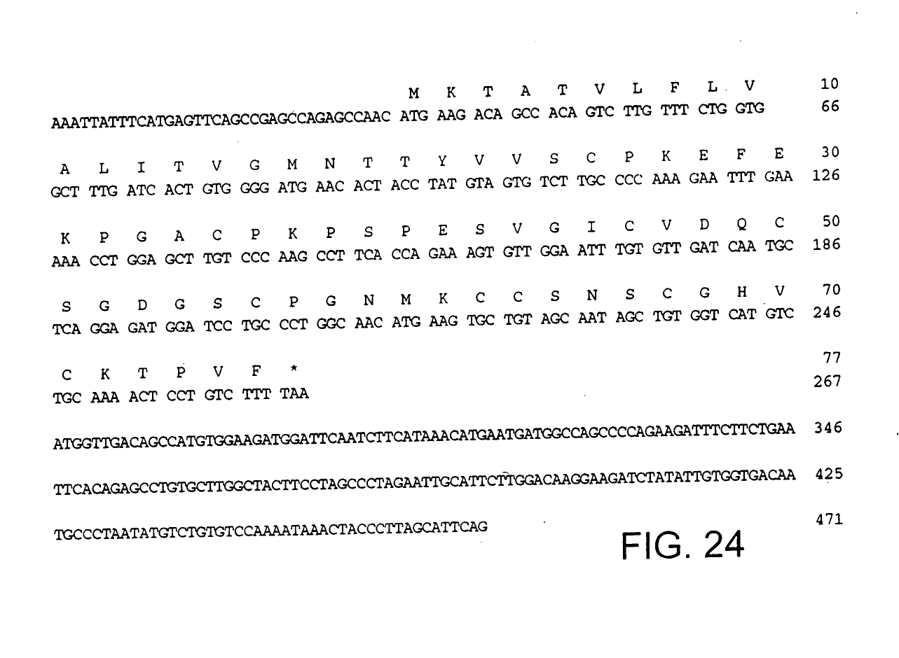

The nucleotide sequence of murine WDNM-2 (FIG. 24; SEQ ID NO:55, SEQ ID NO:57 open reading frame only) is predicted to encode a 75 amino acid protein (SEQ ID NO:56) having a four-disulfide core sequence. The protein is predicted to have a signal sequence extending from amino acid 1 to amino acid 17 of SEQ ID NO:56. The mature protein is predicted to extend from amino acid 18 to amino acid 75 of SEQ ID NO:56 (SEQ ID NO:67). WDNM-2 is likely a serine protease inhibitor. A search for regions with homology to an identified Hidden Markov Motif identified amino acids 31-74 as having homology to PF00095, corresponding Whey Acidic Protein ‘four-disulfide core’.

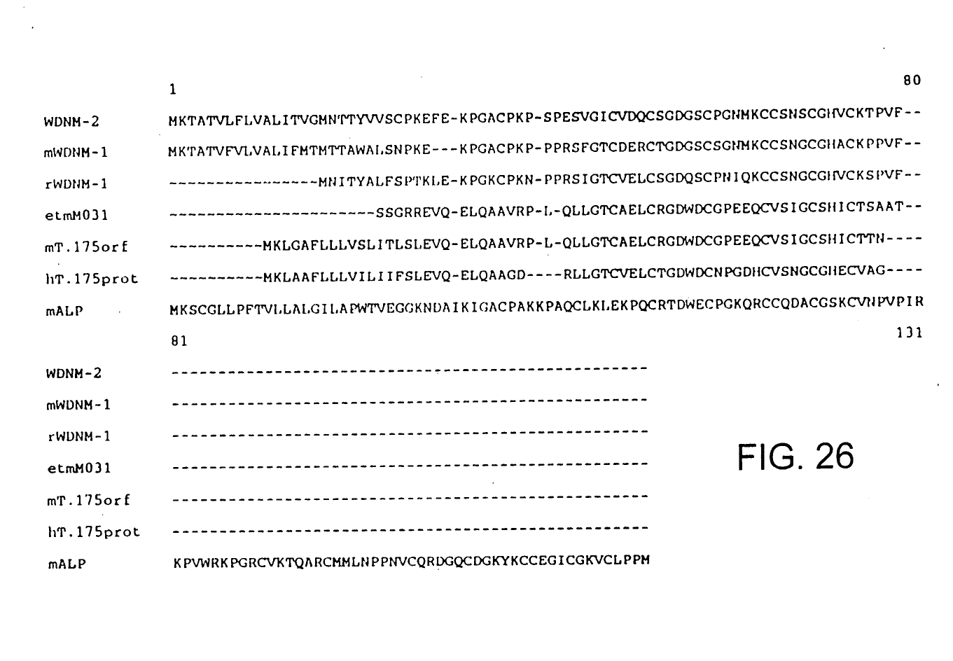

Murine WDNM-2 contains a four-disulfide core pattern of cysteines found in WDNM-1 and related proteins. Thus, murine WDNM-2 protein possesses eight cysteine residues, cysteines C1-C8, which occur at amino acids 35, 46, 50, 56, 62, 63, 67, and 71 of SEQ ID NO:56, respectively. A ninth cysteine residue occurs at amino acid 25. Cysteine residues C1 to C8 are expected to form four interdomain disulfide bonds which stabilize murine WDNM-2 protein. Cysteines C1-C6, C2-C7, C3-C5, and C4-C8 are expected to form disulfide bonds. Like murine and human TANGO-175, murine WDNM-2 protein has some sequence similarity to murine WDNM-1 (mWDNM-1; SEQ ID NO:58), rat WDNM-1 (rWDNM; SEQ ID NO:59), and murine anti-leukoproteinase (mALP; SEQ ID NO:61) (FIG. 26).

The amino acid sequences of murine TANGO-175, human TANGO-175, and murine WDNM-2 bear homology to the amino acid sequences of murine anti-leukoproteinase and WDNM-1. This suggests that TANGO-175 and WDNM-2 have activities similar to that of anti-leukoproteinase and WDNM-1. Thus, TANGO-175 and WDNM-2 may play a functional role similar to that proposed for WDNM-1 by inhibiting proteinases associated with metastasis. TANGO-175 and WDNM-2 may, like murine anti-leukoproteinase, be LPS-induced IFN-gamma suppressible proteins that can inhibit LPS response. Thus, TANGO-175 and WDNM-2 may play a role in regulating inflammation. A functional role for TANGO-175 in inflammation is further suggested by the fact that murine TANGO-175 is highly expressed in the liver during inflammation. TANGO-175 and WDNM-2, like human anti-leukoproteinase (Goselink et al. (1996) J. Exp. Med. 184:1305-1312), may also play a role in the growth of hematopoietic stem cells by neutralizing proteinases produced by bone marrow accessory cells. Accordingly, TANGO-175 and WDNM-2 polypeptides and nucleic acid molecules, anti-TANGO-175 and WDNM-2 antibodies, and modulators of TANGO-175 and WDNM-2 expression or activity may be useful in the treatment and diagnosis of cancer, inflammation, and hematopoietic disorders.

Murine TANGO-175 and WDNM-2 include an Arg-Gly-Asp (RGD) motif. The RGD is present in many proteins which bind to integrins, a group of cell surface receptor proteins which mediate cell attachment. Because integrin-mediated cell attachment influences cell migration, growth, differentiation and apoptosis, among other things, TANGO-175 and WDNM-2 may play a role in such events.

More particularly, the presence of the RGD motif in TANGO-175 and WDNM-2 suggests that TANGO-175 and WDNM-2 may play a role in blood coagulation. For example, TANGO-175 or WDNM-2 (or an RGD motif-containing fragment thereof) may act as an inhibitor of coagulation. Murine TANGO-175, similar to many clotting factors is highly expressed in liver. Thus, the expression pattern of TANGO-175 is consistent with a role in coagulation. Accordingly, TANGO-175 and WDNM-2 polypeptides and nucleic acid molecules, anti-TANGO-175 and anti-WDNM-2 antibodies, and modulators of TANGO-175 or WDNM-2 expression or activity may be useful in the treatment and diagnosis of cancer, inflammation, clotting disorders, and other disorders in which integrin-mediated cell adhesion plays a role.

Accordingly, in one aspect, this invention provides isolated nucleic acid molecules encoding TANGO-175 or WDNM-2 proteins or biologically active portions thereof, as well as nucleic acid fragments suitable for use as primers or hybridization probes for the detection of TANGO-175-encoding nucleic acids or WDNM-2-encoding nucleic acids. As used herein, “TANGO-175”, “TANGO-175 protein” and “TANGO-175 polypeptide” refers to either or both of the human and murine gene products described above as well as homologues of these proteins in other species. As used herein “WDNM-2” refers to the murine gene product described herein as well as homologues in other species.

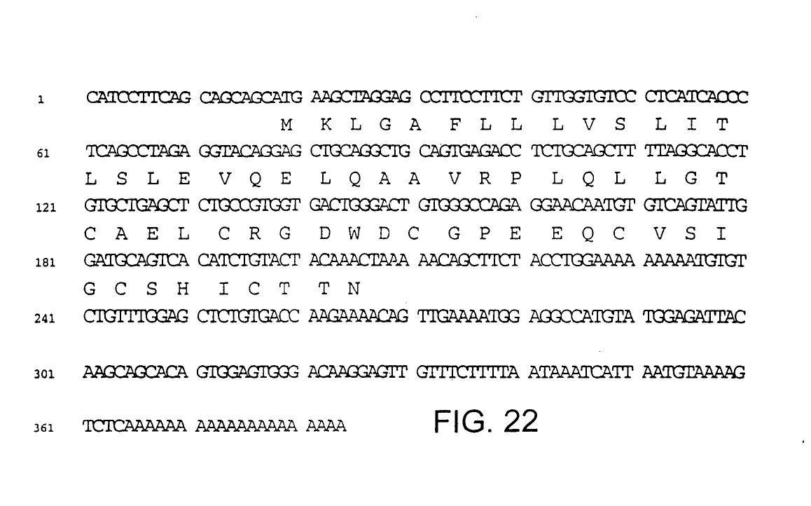

A nucleotide sequence encoding a murine TANGO-175 protein is shown in FIG. 22 (SEQ ID NO:43; SEQ ID NO:45 includes the open reading frame only). The predicted amino acid sequence of murine TANGO-175 is also shown in FIG. 22 (SEQ ID NO:44).

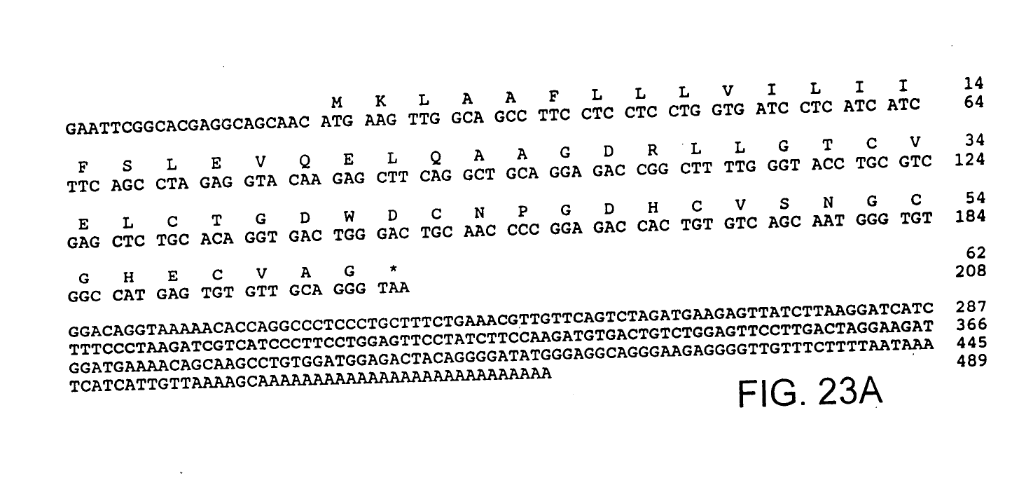

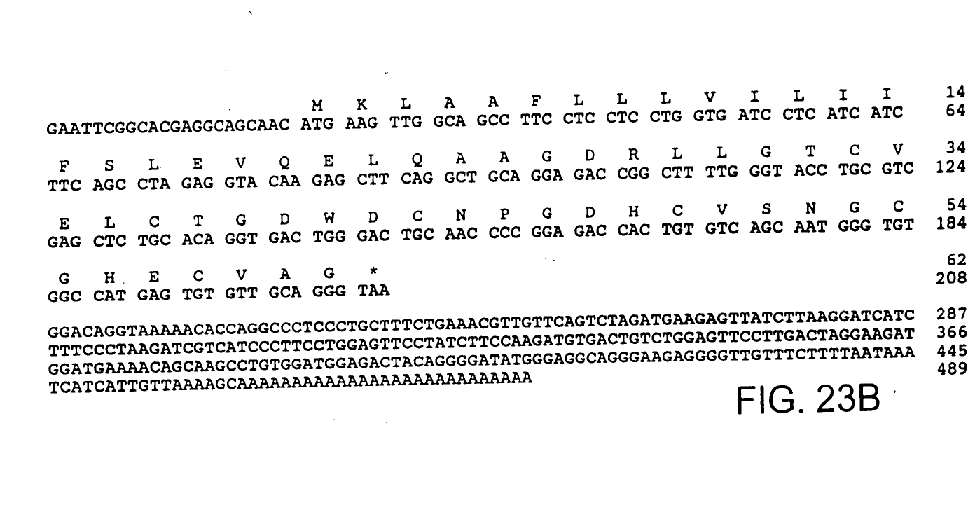

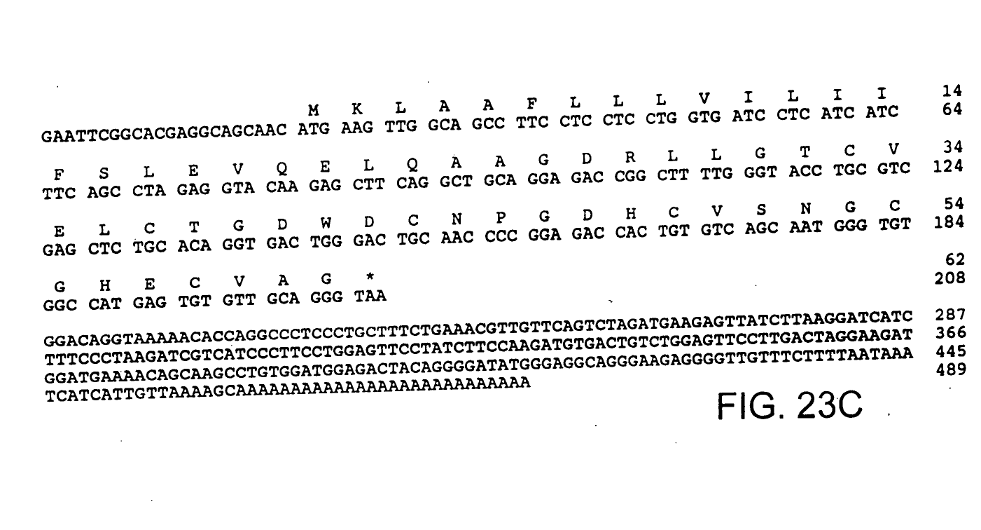

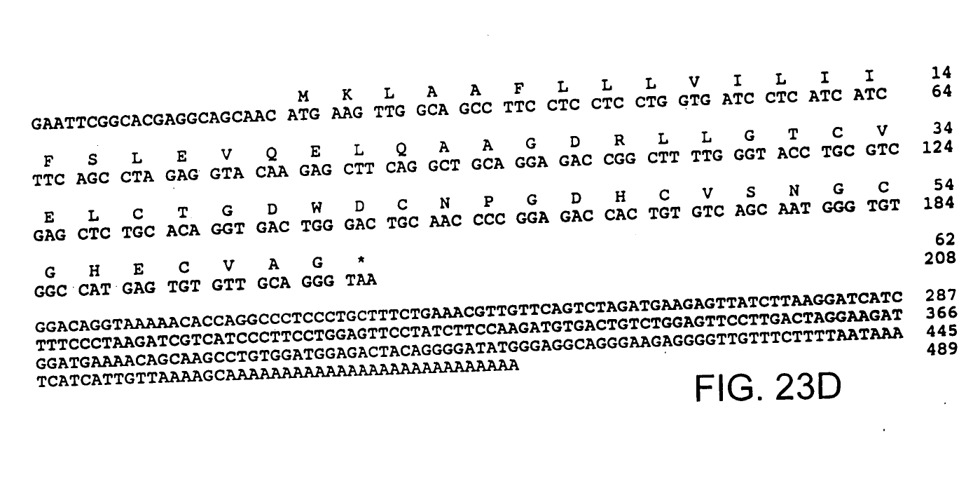

A nucleotide sequence encoding a human TANGO-175 protein is shown in FIGS. 23A-D (SEQ ID NO:46-49; SEQ ID NO:50-53 includes the open reading frame only). The predicted amino acid sequence of human TANGO-175 is also shown in FIGS. 23A-D (SEQ ID NO:54).

A nucleotide sequence encoding murine WDNM-2 is shown in FIG. 24 (SEQ ID NO:55; SEQ ID NO:57 includes the open reading frame only). The predicted amino acid sequence of murine WDNM-2 is also shown in FIG. 24 (SEQ ID NO:56).

A cDNA encoding a portion of murine TANGO-175 was identified in a subtraction library created using stimulated and unstimulated bone marrow cells. The sequence of this partial clone was used to search the IMAGE EST database. This search led to the identification of a clone encoding full-length murine TANGO-175.

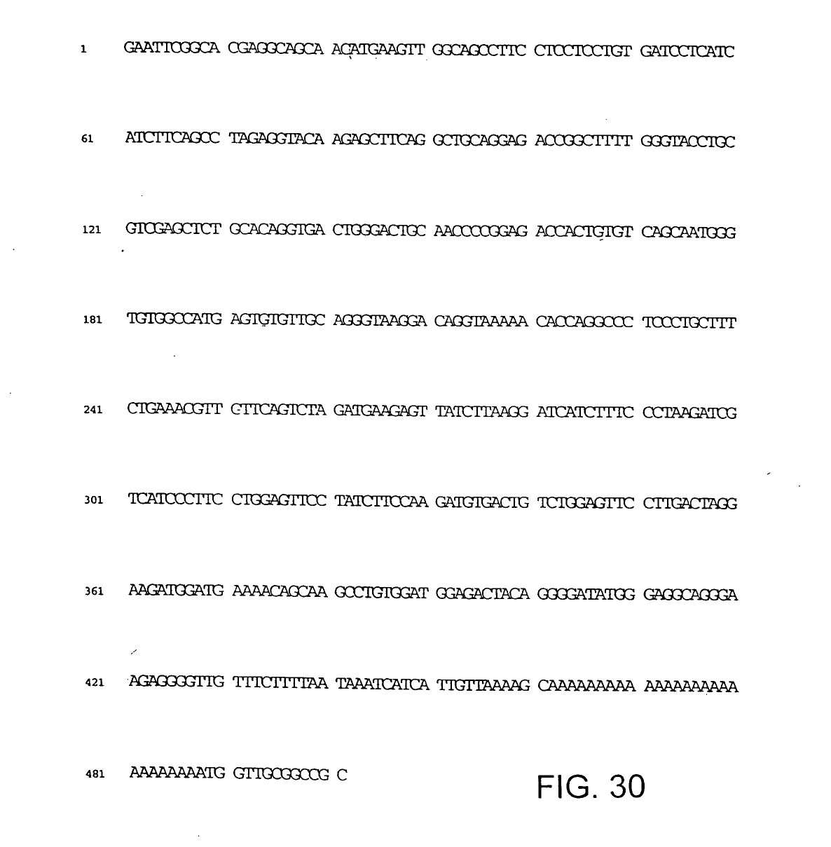

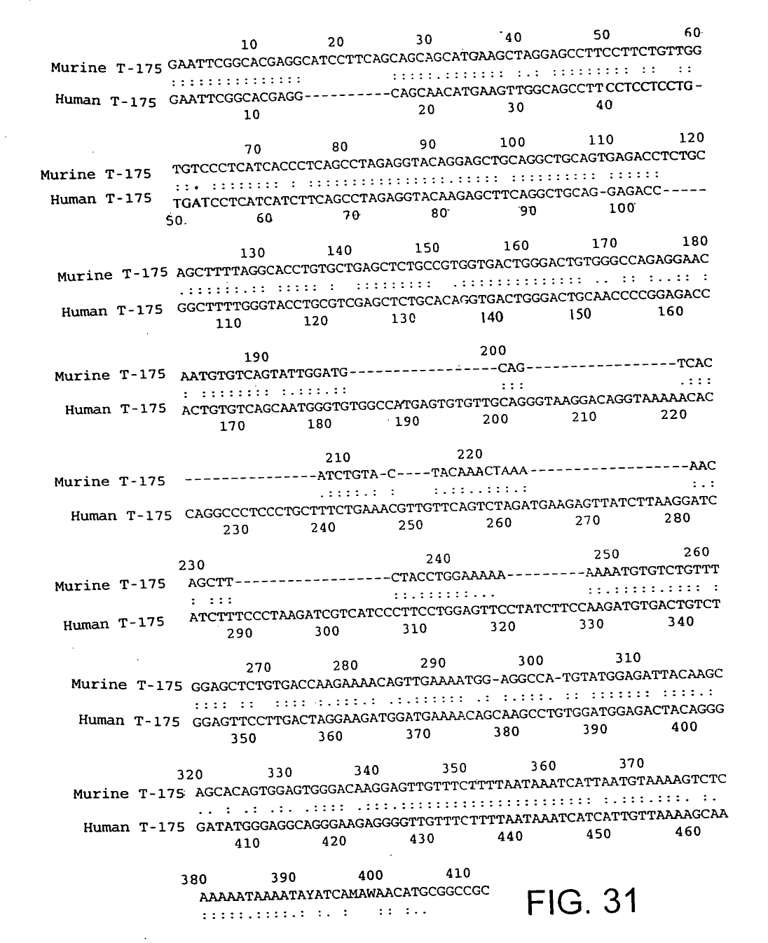

The murine TANGO-175 nucleic sequence was used search the IMAGE EST database in an effort to identify an EST having homology to murine TANGO-175. This search led to the identification of EST W52431. The IMAGE clone corresponding to EST W52431 was obtained and sequenced (SEQ ID NO:62; FIG. 30). The resulting sequence was translated using all three possible reading frames, and the clone does not appear to encode a human homologue of murine TANGO-175. However, analysis of the three potential reading frames for this clone suggested that a change in the reading frame at nucleotide 50, would result in the encoding of a protein, human TANGO-175 (SEQ ID NO:54; FIGS. 23A, 23B, 23C and 23D) with considerable homology to murine TANGO-175 protein.

FIGS. 23A-D depict nucleotide sequences (SEQ ID NOS:46-49; SEQ ID NO: 50-11D, the open reading frame) encoding human TANGO-175 protein. This 501 nucleotide sequence encodes a 61 amino protein having a molecular weight of approximately 4 kDa (excluding post-translational modifications).

Murine WDNM-2 was identified by searching the IMAGE EST database using a composite sequence based on the nucleotide sequences of murine TANGO-175, human TANGO-175, and rat WDNM-1.

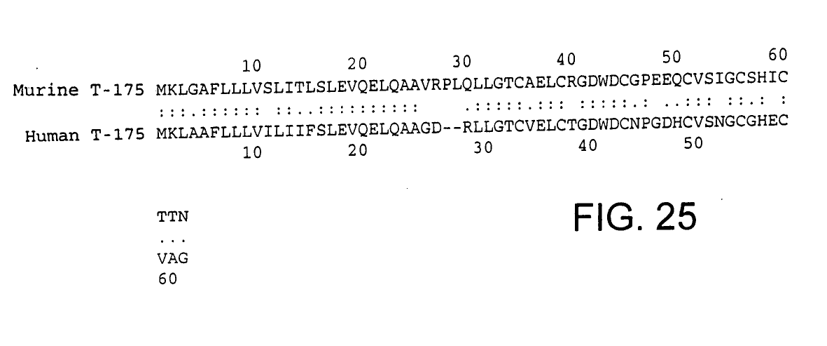

Murine TANGO-175 protein (SEQ ID NO:44), human TANGO-175 protein (SEQ ID NO:54) and murine WDNM-2 bear some similarity to WDNM-1 and anti-leukoproteinase. A sequence alignment of human TANGO-175 (SEQ ID NO:54) and murine TANGO-175 (SEQ ID NO:44) is depicted in FIG. 25. A sequence alignment of murine TANGO-175 (SEQ ID NO:44), human TANGO-175 (SEQ ID NO:54), murine WDNM-2 (SEQ ID NO:56), murine WDNM-1 (SEQ ID NO:58), murine anti-leukoproteinase (SEQ ID NO:61), and rat WDNM-1 (SEQ ID NO:59) is depicted in FIG. 26.

An approximate 0.5 kb murine TANGO-175 mRNA is expressed at a very high level in liver. Much lower level expression of this mRNA is observed in spleen, heart, skeletal muscle, and kidney. An approximate 0.5 kb human TANGO-175 was identified in lymph node, spleen, thymus, uterus, and lung.

Human TANGO-175 is one member of a family of molecules (the “TANGO-175 family”) having certain conserved structural and functional features (e.g., the three disulfide core). The term “family” is defined and described above.

Preferred TANGO-175 polypeptides of the present invention have an amino acid sequence sufficiently identical to the human TANGO-175 amino acid sequence (SEQ ID NO:54). The term “sufficiently identical” is defined and described above.

“Activity”, “biological activity”, and “functional activity” are all defined and described above, and apply in all respects to T175. A TANGO-175 activity can be a direct activity, such as an association with or an enzymatic activity on a second protein or an indirect activity, such as a cellular adhesion activity mediated by interaction of the TANGO-175 protein with a second protein.

Another aspect of this invention features isolated or recombinant TANGO-175 proteins and polypeptides. Preferred TANGO-175 proteins and polypeptides possess at least one biological activity possessed by naturally occurring human TANGO-175, e.g., (1) the ability to form protein:protein interactions with a protein that naturally binds TANGO-175; (2) the ability to bind a TANGO-175 receptor, e.g., an integrin; (3) the ability to inhibit a proteinase activity; (4) the ability to modulate cell-cell interactions; (5) the ability to modulate hematopoiesis (e.g., the ability to modulate proliferation, differentiation or function of hematopoietic cells, e.g., stem cells); (6) the ability to modulate of inflammation, and (7) the ability to modulate intravasation and/or extravasation; (8) the ability to modulate clotting; and (a) the ability to modulate cell proliferation. Accordingly, another embodiment of the invention features isolated TANGO-175 proteins and polypeptides having at least one TANGO-175 activity.

Yet another embodiment of the invention features TANGO-175 molecules that contain a signal sequence. “Signal sequence” is defined and described above. The native human TANGO-175 signal sequence or signal peptide can be removed and replaced with a signal sequence from another protein. In certain host cells (e.g., mammalian host cells), expression and/or secretion of TANGO-175 can be increased through use of a heterologous signal sequence. For example, the gp67 secretory sequence of the baculovirus envelope protein can be used as a heterologous signal sequence in expression systems, e.g., to facilitate the secretion of a protein of interest.

Human WDNM-2 is one member of a family of molecules (the “WDNM-2 family”) having certain conserved structural and functional features (e.g., the three disulfide core). The term “family” is defined and described above.

Preferred WDNM-2 polypeptides of the present invention have an amino acid sequence sufficiently identical to the human WDNM-2 amino acid sequence (SEQ ID NO:54). The term “sufficiently identical” is defined and described above.

“Activity”, “biological activity”, and “functional activity” are all defined and described above, and apply in all respects to WDNM-2. A WDNM-2 activity can be a direct activity, such as an association with or an enzymatic activity on a second protein or an indirect activity, such as a cellular adhesion activity mediated by interaction of the WDNM-2 protein with a second protein.

Another aspect of this invention features isolated or recombinant WDNM-2 proteins and polypeptides. Preferred WDNM-2 proteins and polypeptides possess at least one biological activity possessed by naturally occurring human WDNM-2, e.g., (1) the ability to form protein:protein interactions with a protein that naturally binds WDNM-2; (2) the ability to bind a WDNM-2 receptor, e.g., an integrin; (3) the ability to inhibit a proteinase activity; (4) the ability to modulate cell-cell interactions; (5) the ability to modulate hematopoiesis (e.g., the ability to modulate proliferation of hematopoietic stem cells); (6) the ability to modulate of inflammation, and (7) the ability to modulate intravasation and/or extravasation; (8) the ability to modulate clotting; and (a) the ability to modulate cell proliferation. Accordingly, another embodiment of the invention features isolated WDNM-2 proteins and polypeptides having at least one WDNM-2 activity.

Yet another embodiment of the invention features WDNM-2 molecules which contains a signal sequence. “Signal sequence” is defined and described above. The native human WDNM-2 signal sequence or signal peptide can be removed and replaced with a signal sequence from another protein. In certain host cells (e.g., mammalian host cells), expression and/or secretion of WDNM-2 can be increased through use of a heterologous signal sequence. For example, the gp67 secretory sequence of the baculovirus envelope protein can be used as a heterologous signal sequence in expression systems, e.g., to facilitate the secretion of a protein of interest.

Various aspects of the invention are described in further detail in the following subsections.

Isolated Nucleic Acid Molecules

One aspect of the invention pertains to isolated nucleic acid molecules that encode polypeptides of the invention or biologically active portions thereof. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid. Preferably, an “isolated” nucleic acid is free of sequences (preferably protein encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecules of the invention can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the terms “gene” and “recombinant gene” refer to nucleic acid molecules comprising an open reading frame encoding a polypeptide of the invention, preferably a mammalian polypeptide of the invention. A nucleic acid molecule of the present invention, e.g., a nucleic acid molecule having the nucleotide sequence of SEQ ID NO:46, SEQ ID NO:47, SEQ ID NO:48, SEQ ID NO:49, SEQ ID NO:50, SEQ ID NO:51, SEQ ID NO:52, SEQ ID NO:53, or a complement of any of these nucleotide sequences, can be isolated using standard molecular biology techniques and the sequence information provided herein. For example, using all or a portion of the nucleic acid sequences of SEQ ID NO:46, SEQ ID NO:47, SEQ ID NO:48, SEQ ID NO:49, SEQ ID NO:50, SEQ ID NO:51, SEQ ID NO:52, SEQ ID NO:53, as a hybridization probe, nucleic acid molecules of the invention can be isolated using standard hybridization and cloning techniques (e.g., as described in Sambrook et al., eds., Molecular Cloning: A Laboratory Manual, 2nd ed., Cold Spring Harbor Laboratory, Cold Spring Harbor Laboratory Press, Cold Spring Harbor, N.Y., 1989).

A nucleic acid of the invention can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to nucleotide sequences of nucleic acids of the invention can be prepared by standard synthetic techniques, e.g., using an automated DNA synthesizer.

In another preferred embodiment, an isolated nucleic acid molecule of the invention comprises a nucleic acid molecule which is a complement of the nucleotide sequence shown in SEQ ID NO:1, SEQ ID NO:3, SEQ ID NO:9, SEQ ID NO:11, SEQ ID NO:29, SEQ ID NO:31, SEQ ID NO:40, SEQ ID NO:46, SEQ ID NO:47, SEQ ID NO:48, SEQ ID NO:49, SEQ ID NO:50, SEQ ID NO:51, SEQ ID NO:52, SEQ ID NO:53, or a portion thereof. A nucleic acid molecule which is complementary to a given nucleotide sequence is one which is sufficiently complementary to the given nucleotide sequence that it can hybridize to the given nucleotide sequence thereby forming a stable duplex.

Moreover, the nucleic acid molecules of the invention can comprise only a portion of a nucleic acid sequence encoding polypeptides of the invention, for example, a fragment which can be used as a probe or primer or a fragment encoding a biologically active portion of polypetides of the invention. The nucleotide sequences determined from the cloning of the human genes of the invention allow for the generation of probes and primers designed for use in identifying and/or cloning homologues of nucleic acids of the invention in other cell types, e.g., from other tissues, as well as homologues of nucleic acids of the invention from other mammals. The probe/primer typically comprises substantially purified oligonucleotide. The oligonucleotide typically comprises a region of nucleotide sequence that hybridizes under stringent conditions to at least about 12, preferably about 25, more preferably about 50, 75, 100, 125, 150, 175, 200, 250, 300, or 350 consecutive nucleotides of the sense or anti-sense sequence of, for example, SEQ ID NO:1, SEQ ID NO:3, SEQ ID NO:9, SEQ ID NO:11, SEQ ID NO:29, SEQ ID NO:31, SEQ ID NO:40, SEQ ID NO:46, SEQ ID NO:47, SEQ ID NO:48, SEQ ID NO:49, SEQ ID NO:50, SEQ ID NO:51, SEQ ID NO:52, SEQ ID NO:53, or of a naturally occurring mutant of, for example, SEQ ID NO:1, SEQ ID NO:3, SEQ ID NO:9, SEQ ID NO:11, SEQ ID NO:29, SEQ ID NO:31, SEQ ID NO:40, SEQ ID NO:46, SEQ ID NO:47, SEQ ID NO:48, SEQ ID NO:49, SEQ ID NO:50, SEQ ID NO:51, SEQ ID NO:52, or SEQ ID NO:53.

Probes based on the nucleotide sequences of nucleic acids of the invention can be used to detect transcripts or genomic sequences encoding similar or identical proteins. The probe comprises a label group attached thereto, e.g., a radioisotope, a fluorescent compound, an enzyme, or an enzyme co-factor. Such probes can be used as a part of a diagnostic test kit for identifying cells or tissues which mis-express a polypeptide of the invention, such as by measuring a level of a nucleic acid encoding a polypeptide of the invention in a sample of cells from a subject, e.g., detecting levels of mRNA of the invention or determining whether a genomic gene of the invention has been mutated or deleted.

A nucleic acid fragment encoding a “biologically active portion of a polypeptide of the invention” can be prepared by isolating a portion of, for example, SEQ ID NO:1, SEQ ID NO:3, SEQ ID NO:9, SEQ ID NO:11, SEQ ID NO:29, SEQ ID NO:31, SEQ ID NO:40, SEQ ID NO:46, SEQ ID NO:47, SEQ ID NO:48, SEQ ID NO:49, SEQ ID NO:50, SEQ ID NO:51, SEQ ID NO:52, or SEQ ID NO:53, which encodes a polypeptide having a biological activity of a polypeptide of the invention, expressing the encoded portion of a polypeptide of the invention (e.g., by recombinant expression in vitro) and assessing the activity of the encoded portion of a polypeptide of the invention.

The invention further encompasses nucleic acid molecules that differ from the nucleotide sequence of, for example, SEQ ID NO:1, SEQ ID NO:3, SEQ ID NO:9, SEQ ID NO:11, SEQ ID NO:29, SEQ ID NO:31, SEQ ID NO:40, SEQ ID NO:46, SEQ ID NO:47, SEQ ID NO:48, SEQ ID NO:49, SEQ ID NO:50, SEQ ID NO:51, SEQ ID NO:52, or SEQ ID NO:53, due to degeneracy of the genetic code and thus encode the same polypeptide of the invention as that encoded by the nucleotide sequence shown in, for example, SEQ ID NO:1, SEQ ID NO:3, SEQ ID NO:9, SEQ ID NO:11, SEQ ID NO:29, SEQ ID NO:31, SEQ ID NO:40, SEQ ID NO:46, SEQ ID NO:47, SEQ ID NO:48, SEQ ID NO:49, SEQ ID NO:50, SEQ ID NO:51, SEQ ID NO:52, or SEQ ID NO:53.

In addition to the nucleotide sequences of the nucleic acids of the invention shown in, for example, SEQ ID NO:1, SEQ ID NO:3, SEQ ID NO:9, SEQ ID NO:11, SEQ ID NO:29, SEQ ID NO:31, SEQ ID NO:40, SEQ ID NO:46, SEQ ID NO:47, SEQ ID NO:48, SEQ ID NO:49, SEQ ID NO:50, SEQ ID NO:51, SEQ ID NO:52, or SEQ ID NO:53, it will be appreciated by those skilled in the art that DNA sequence polymorphisms that lead to changes in the amino acid sequences of polypeptides of the invention may exist within a population (e.g., the human population). Such genetic polymorphism in the genes of the invention may exist among individuals within a population due to natural allelic variation. An allele is one of a group of genes which occur alternatively at a given genetic locus. Such natural allelic variations can typically result in 1-5% variance in the nucleotide sequence of the genes of the invention. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms in polypeptides of the invention that are the result of natural allelic variation and that do not alter the functional activity of polypeptides of the invention are intended to be within the scope of the invention.

Moreover, nucleic acid molecules encoding polypeptides of the invention from other species (TANGO-139, 125, 110, 175, or WDNM-2 homologues), which have a nucleotide sequence which differs from that of a human nucleic acid of the invention, are intended to be within the scope of the invention. Nucleic acid molecules corresponding to natural allelic variants and homologues of the cDNA of the invention can be isolated based on their identity to the human nucleic acids of the invention disclosed herein using the human cDNAs, or a portion thereof, as a hybridization probe according to standard hybridization techniques under stringent hybridization conditions. For example, splice variants of human and mouse cDNA of the invention can be isolated based on identity to human and mouse nucleic acids of the invention.

As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2 X SSC, 0.1% SDS at 50-65° C. Preferably, an isolated nucleic acid molecule of the invention that hybridizes under stringent conditions to the coding or non-coding (or “sense” or “anti-sense”) sequence of, for example, SEQ ID NO:1, SEQ ID NO:3, SEQ ID NO:9, SEQ ID NO:11, SEQ ID NO:29, SEQ ID NO:31, SEQ ID NO:40, SEQ ID NO:46, SEQ ID NO:47, SEQ ID NO:48, SEQ ID NO:49, SEQ ID NO:50, SEQ ID NO:51, SEQ ID NO:52, or SEQ ID NO:53 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the nucleotide sequence of nucleic acids of the invention that may exist in the population, the skilled artisan will further appreciate that changes can be introduced by mutation into the nucleotide sequence of, for example, SEQ ID NO:1, SEQ ID NO:3, SEQ ID NO:9, SEQ ID NO:11, SEQ ID NO:29, SEQ ID NO:31, SEQ ID NO:40, SEQ ID NO:46, SEQ ID NO:47, SEQ ID NO:48, SEQ ID NO:49, SEQ ID NO:50, SEQ ID NO:51, SEQ ID NO:52, or SEQ ID NO:53, thereby leading to changes in the amino acid sequence of the encoded polypeptides of the invention, without altering the biological ability of the polypeptides of the invention. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a polypeptide of the invention is preferably replaced with another amino acid residue from the same side chain family. Alternatively, mutations can be introduced randomly along all or part of a coding sequence of a nucleic acid of the invention, such as by saturation mutagenesis, and the resultant mutants can be screened for biological activity of polypeptides of the invention biological activity to identify mutants that retain activity. Following mutagenesis, the encoded protein can be expressed recombinantly and the activity of the protein can be determined.

In order to avoid severely reducing or eliminating biological activity, amino acid residues that are conserved among the polypeptides of the invention of various species are not altered (except by conservative substitution).

Conserved domains and cysteine residues are less likely to be amenable to mutation. Other amino acid residues, however, (e.g., those that are not conserved or only semi-conserved among polypeptides of the invention of various species e.g., between murine and human polypeptides of the invention) may not be essential for activity and thus are likely to be amenable to alteration.

Accordingly, another aspect of the invention pertains to nucleic acid molecules encoding polypeptides of the invention that contain changes in amino acid residues that are not essential for activity. Such polypeptides of the invention differ in amino acid sequence from, for example, SEQ ID NO:2, SEQ ID NO:4, SEQ ID NO:10, SEQ ID NO:12, SEQ ID NO:30, SEQ ID NO:32, SEQ ID NO:42, SEQ ID NO:54, or SEQ ID NO:64 yet retain biological activity. In one embodiment, the isolated nucleic acid molecule includes a nucleotide sequence encoding a protein that includes an amino acid sequence that is at least about 45% identical, 65%, 75%, 85%, 95%, or 98% identical to the amino acid sequence of, for example, SEQ ID NO:2, SEQ ID NO:4, SEQ ID NO:10, SEQ ID NO:12, SEQ ID NO:30, SEQ ID NO:32, SEQ ID NO:42, SEQ ID NO:54, or SEQ ID NO:64.

An isolated nucleic acid molecule encoding a polypeptide of the invention having a sequence which differs from that of, for example, SEQ ID NO:2, SEQ ID NO:4, SEQ ID NO:10, SEQ ID NO:12, SEQ ID NO:30, SEQ ID NO:32, SEQ ID NO:42, SEQ ID NO:54, or SEQ ID NO:64 can be created by introducing one or more nucleotide substitutions, additions or deletions into the nucleotide sequence of, for example, SEQ ID NO:1, SEQ ID NO:3, SEQ ID NO:9, SEQ ID NO:11, SEQ ID NO:29, SEQ ID NO:31, SEQ ID NO:40, SEQ ID NO:46, SEQ ID NO:47, SEQ ID NO:48, SEQ ID NO:49, SEQ ID NO:50, SEQ ID NO:51, SEQ ID NO:52, or SEQ ID NO:53, such that one or more amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. “Conservative amino acid substitution” is defined and described above.

Another aspect of this invention features isolated or recombinant polypeptides of the invention. Preferred polypeptides of the invention possess at least one of the following biological activities possessed by naturally occurring human polypeptides of the invention: (1) the ability to form protein:protein interactions with proteins; (2) the ability to bind a ligand; (3) the ability to bind a receptor; (4) ability to modulate cellular proliferation; and (5) ability to modulate cellular differentiation.

The invention also features T110 that, in addition to those listed above, possesses at least one of the following biological activities: (1) the ability to bind to an intracellular target protein; and (2) the ability to interact with a protein involved in cellular proliferation or differentiation.

The invention also features T175 that, in addition to those listed above, possesses at least one of the following biological activities: (1) the ability to inhibit a proteinase activity; (2) the ability to modulate cell-cell interactions; (3) the ability to modulate hematopoiesis (e.g., the ability to modulate proliferation of hematopoietic stem cells; (4) the ability to modulate inflammation; (5) the ability to modulate intravasation and/or extravasation; (6) the ability to modulate clotting.

The present invention encompasses antisense nucleic acid molecules, i.e., molecules which are complementary to a sense nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand of a nucleic acid of the invention, or to only a portion thereof, e.g., all or part of the protein coding region (or open reading frame). An antisense nucleic acid molecule can be antisense to a noncoding region of the coding strand of a nucleotide sequence encoding a polypeptide of the invention. The noncoding regions (“5′ and 3′ untranslated regions”) are the 5′ and 3′ sequences which flank the coding region and are not translated into amino acids.

Given the coding strand sequences encoding polypeptides of the invention disclosed herein (e.g., SEQ ID NO:1, SEQ ID NO:3, SEQ ID NO:9, SEQ ID NO:11, SEQ ID NO:29, SEQ ID NO:31, SEQ ID NO:40, SEQ ID NO:46, SEQ ID NO:47, SEQ ID NO:48, SEQ ID NO:49, SEQ ID NO:50, SEQ ID NO:51, SEQ ID NO:52, or SEQ ID NO:53), antisense nucleic acids of the invention can be designed according to the rules of Watson and Crick base pairing. The antisense nucleic acid molecule can be complementary to the entire coding region of mRNA of the invention, but more preferably is an oligonucleotide which is antisense to only a portion of the coding or noncoding region of human mRNA of the invention. For example, the antisense oligonucleotide can be complementary to the region surrounding the translation start site of mRNA of the invention. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a polypeptide of the invention to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave mRNA transcripts of the invention to thereby inhibit translation of mRNA of the invention. A ribozyme having specificity for a nucleic acid encoding a polypeptide of the invention can be designed based upon the nucleotide sequence of a cDNA of the invention disclosed herein (e.g., SEQ ID NO:1, SEQ ID NO:3, SEQ ID NO:9, SEQ ID NO:11, SEQ ID NO:29, SEQ ID NO:31, SEQ ID NO:40, SEQ ID NO:46, SEQ ID NO:47, SEQ ID NO:48, SEQ ID NO:49, SEQ ID NO:50, SEQ ID NO:51, SEQ ID NO:52, or SEQ ID NO:53). For example, a derivative of a Tetrahymena L-19 IVS RNA can be constructed in which the nucleotide sequence of the active site is complementary to the nucleotide sequence to be cleaved in an mRNA encoding a polypeptide of the invention. See, e.g., Cech et al. U.S. Pat. No. 4,987,071; and Cech et al. U.S. Pat. No. 5,116,742. Alternatively, mRNA of the invention can be used to select a catalytic RNA having a specific ribonuclease activity from a pool of RNA molecules. See, e.g., Bartel and Szostak (1993) Science 261:1411-1418.

The invention also encompasses nucleic acid molecules which form triple helical structures. For example, expression of a gene of the invention can be inhibited by targeting nucleotide sequences complementary to the regulatory region of the gene of the invention (e.g., the TANGO-139, 125, 110, 175, or WDNM-2 promoters and/or enhancers) to form triple helical structures that prevent transcription of the gene of the invention in target cells. See generally, Helene (1991) Anticancer Drug Des. 6(6):569-84; Helene (1992) Ann. N.Y. Acad. Sci. 660:27-36; and Maher (1992) Bioassays 14(12):807-15.

In preferred embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al. (1996) Proc. Natl. Acad. Sci. USA 93: 14670-675.

PNAs of nucleic acids of the invention can be used in therapeutic and diagnostic applications. For example, PNAs can be used as antisense or antigene agents for sequence-specific modulation of gene expression by, e.g., inducing transcription or translation arrest or inhibiting replication. PNAs of nucleic acids of the invention can also be used, e.g., in the analysis of single base pair mutations in a gene by, e.g., PNA directed PCR clamping; as artificial restriction enzymes when used in combination with other enzymes, e.g., S1 nucleases (Hyrup (1996) supra; or as probes or primers for DNA sequence and hybridization (Hyrup (1996) supra; Perry-O'Keefe et al. (1996) Proc. Natl. Acad. Sci. USA 93: 14670-675).

In another embodiment, PNAs of nucleic acids of the invention can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of nucleic acids of the invention can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) supra). The synthesis of PNA-DNA chimeras can be performed as described in Hyrup (1996) supra and Finn et al. (1996) Nucleic Acids Research 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al. (1989) Nucleic Acid Res. 17:5973-88). PNA monomers are then coupled in a stepwise manner to produce a chimeric molecule with a 5′ PNA segment and a 3′ DNA segment (Finn et al. (1996) Nucleic Acids Res. 24(17):3357-63). Alternatively, chimeric molecules can be synthesized with a 5′ DNA segment and a 3′ PNA segment (Peterser et al. (1975) Bioorganic Med. Chem. Lett. 5:1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide may be conjugated to another molecule, e.g., a peptide, hybridization triggered cross-linking agent, transport agent, hybridization-triggered cleavage agent, etc.

Isolated TANGO-139, 125, 110, 175 or WDNM-2 Proteins and Anti-TANGO-139, 125, 110, 175, or WDNM-2 Antibodies

One aspect of the invention pertains to isolated polypeptides of the invention, and biologically active portions thereof, as well as polypeptide fragments suitable for use as immunogens to raise anti-polypeptides-of-the-invention antibodies. In one embodiment, native polypeptides of the invention can be isolated from cells or tissue sources by an appropriate purification scheme using standard protein purification techniques. In another embodiment, polypeptides of the invention are produced by recombinant DNA techniques. Alternative to recombinant expression, a polypeptide of the invention can be synthesized chemically using standard peptide synthesis techniques.

An “isolated” or “purified” protein or biologically active portion thereof is substantially free of cellular material or other contaminating proteins from the cell or tissue source from which the polypeptide of the invention is derived, or substantially free of chemical precursors or other chemicals when chemically synthesized. The language “substantially free of cellular material” includes preparations of polypeptide of the invention in which the protein is separated from cellular components of the cells from which it is isolated or recombinantly produced. Thus, a polypeptide of the invention that is substantially free of cellular material includes preparations of a polypeptide of the invention having less than about 30%, 20%, 10%, or 5% (by dry weight) of polypeptide not of the invention (also referred to herein as a “contaminating protein”). When the polypeptide of the invention or biologically active portion thereof is recombinantly produced, it is also preferably substantially free of culture medium, i.e., culture medium represents less than about 20%, 10%, or 5% of the volume of the protein preparation. When polypeptide of the invention is produced by chemical synthesis, it is preferably substantially free of chemical precursors or other chemicals, i.e., it is separated from chemical precursors or other chemicals which are involved in the synthesis of the protein. Accordingly such preparations of polypeptide of the invention have less than about 30%, 20%, 10%, 5% (by dry weight) of chemical precursors or chemicals not of the invention.

Biologically active portions of a polypeptide of the invention include peptides comprising amino acid sequences sufficiently identical to or derived from the amino acid sequence of a polypeptide of the invention (e.g., the amino acid sequence shown in SEQ ID NO:2, SEQ ID NO:4, SEQ ID NO:10, SEQ ID NO:12, SEQ ID NO:30, SEQ ID NO:32, SEQ ID NO:42, SEQ ID NO:54, or SEQ ID NO:64), which include fewer amino acids than the full length polypeptides of the invention, and exhibit at least one activity of a polypeptide of the invention. Typically, biologically active portions comprise a domain or motif with at least one activity of the polypeptide of the invention. A biologically active portion of a polypeptide of the invention can be a polypeptide which is, for example, 10, 25, 50, 60, or more amino acids in length.

Moreover, other biologically active portions, in which other regions of the protein are deleted, can be prepared by recombinant techniques and evaluated for one or more of the functional activities of a native polypeptide of the invention.

Preferred polypeptide of the invention has the amino acid sequence of SEQ ID NO:2, SEQ ID NO:4, SEQ ID NO:10, SEQ ID NO:12, SEQ ID NO:30, SEQ ID NO:32, SEQ ID NO:42, SEQ ID NO:54, or SEQ ID NO:64. Other useful polypeptides of the invention are substantially identical to SEQ ID NO:2, SEQ ID NO:4, SEQ ID NO:10, SEQ ID NO:12, SEQ ID NO:30, SEQ ID NO:32, SEQ ID NO:42, SEQ ID NO:54, or SEQ ID NO:64 and retain the functional activity of the protein of SEQ ID NO:2, SEQ ID NO:4, SEQ ID NO:10, SEQ ID NO:12, SEQ ID NO:30, SEQ ID NO:32, SEQ ID NO:42, SEQ ID NO:54, or SEQ ID NO:64, yet differ in amino acid sequence due to natural allelic variation or mutagenesis.

Accordingly, a useful polypeptide of the invention is a protein which includes an amino acid sequence at least about 45%, preferably 55%, 65%, 75%, 85%, 95%, or 99% identical to the amino acid sequence of SEQ ID NO:2, SEQ ID NO:4, SEQ ID NO:10, SEQ ID NO:12, SEQ ID NO:30, SEQ ID NO:32, SEQ ID NO:42, SEQ ID NO:54, or SEQ ID NO:64 and retains the functional activity of the polypeptides of the invention of SEQ ID NO:2, SEQ ID NO:4, SEQ ID NO:10, SEQ ID NO:12, SEQ ID NO:30, SEQ ID NO:32, SEQ ID NO:42, SEQ ID NO:54, or SEQ ID NO:64. In a preferred embodiment, the polypeptide of the invention retains a functional activity of the polypeptide of the invention of SEQ ID NO:2, SEQ ID NO:10, SEQ ID NO:30, or SEQ ID NO:54.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping)×100). Preferably, the two sequences are the same length.

The determination of percent homology between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403410. BLAST nucleotide searches can be performed with the NBLAST program, score=100, wordlength=12 to obtain nucleotide sequences homologous to TANGO-175 nucleic acid molecules of the invention. BLAST protein searches can be performed with the XBLAST program, score=50, wordlength=3 to obtain amino acid sequences homologous to polypeptides of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, CABIOS 4:11-17 (1988). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Another preferred, non-limiting example of a mathematical algorithm utilized for the comparison of sequences is the local homology algorithm of Smith and Waterman (Advances in Applied Mathematics 2: 482-489 (1981)). Such an algorithm is incorporated into the BestFit program, which is part of the Wisconsin™ package, and is used to find the best segment of similarity between two sequences. BestFit reads a scoring matrix that contains values for every possible GCG symbol match. The program uses these values to construct a path matrix that represents the entire surface of comparison with a score at every position for the best possible alignment to that point. The quality score for the best alignment to any point is equal to the sum of the scoring matrix values of the matches in that alignment, less the gap creation penalty multiplied by the number of gaps in that alignment, less the gap extension penalty multiplied by the total length of all gaps in that alignment. The gap creation and gap extension penalties are set by the user. If the best path to any point has a negative value, a zero is put in that position.

After the path matrix is complete, the highest value on the surface of comparison represents the end of the best region of similarity between the sequences. The best path from this highest value backwards to the point where the values revert to zero is the alignment shown by BestFit. This alignment is the best segment of similarity between the two sequences. Further documentation can be found at http://ir.ucdavis.edu/GCGhelp/bestfit.html#algorithm.

Additional algorithms for sequence analysis are known in the art and include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. For a further description of FASTA parameters, see http://bioweb.pasteur.fr/docs/man/man/fasta.1.html#sect2, the contents of which are incorporated herein by reference.

The percent identity between two sequences can be determined using techniques similar to those described above, with or without allowing gaps. In calculating percent identity, typically exact matches are counted.

As used herein, the phrase “allelic variant” refers to a nucleotide sequence which occurs at a given locus or to a polypeptide encoded by the nucleotide sequence. For example, TANGO 125 gene exhibits significant homology with GENBANK™ entry gi-1841553. Allelic variants of any of these genes can be identified by sequencing the corresponding chromosomal portion at the indication location in multiple individuals.

The percent identity between two sequences can be determined using techniques similar to those described above, with or without allowing gaps. In calculating percent identity, only exact matches are counted.

The invention also provides polypeptides of the invention that are chimeric or fusion proteins. As used herein, a polypeptide of the invention that is a “chimeric protein” or “fusion protein” comprises a polypeptide of the invention operably linked to a polypeptide not of the invention. A “polypeptide of the invention” refers to a polypeptide having an amino acid sequence corresponding to a polypeptide of the invention, whereas a “polypeptide not of the invention” refers to a polypeptide having an amino acid sequence corresponding to a protein which is not substantially identical to a polypeptide of the invention, e.g., a protein which is different from the polypeptides of the invention and which is derived from the same or a different organism. Within a fusion protein of the invention the polypeptide of the invention can correspond to all or a portion of a polypeptide of the invention, preferably at least one biologically active portion of a polypeptide of the invention. Within the fusion protein, the term “operably linked” is intended to indicate that the polypeptide of the invention and the polypeptide not of the invention are fused in-frame to each other. The polypeptide not of the invention can be fused to the N-terminus or C-terminus of the polypeptide of the invention.

One useful fusion protein is a GST-polypeptide-of-the-invention fusion protein in which the sequences of polypeptides of the invention are fused to the C-terminus of the GST sequences. Such fusion proteins can facilitate the purification of recombinant nucleic acids or polypeptides of the invention.

In another embodiment, the fusion protein is a polypeptide of the invention containing a heterologous signal sequence at its N-terminus. For example, the native signal sequence of a polypeptide of the invention (e.g., about amino acids 1 to 25 of SEQ ID NO:54) can be removed and replaced with a signal sequence from another protein. In certain host cells (e.g., mammalian host cells), expression and/or secretion of a polypeptide of the invention can be increased through use of a heterologous signal sequence. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Molecular cloning, Sambrook et al., supra) and the protein A secretory signal (Pharmacia Biotech; Piscataway, N.J.).

In yet another embodiment, the fusion protein is a fusion protein of immunoglobin and a polypeptide of the invention in which all or part of a polypeptide of the invention is fused to sequences derived from a member of the immunoglobulin protein family. The fusion protein of immunoglobin and a polypeptide of the invention that are part of the invention can be incorporated into pharmaceutical compositions and administered to a subject to inhibit an interaction between a ligand of a polypeptide of the invention and a polypeptide of the invention on the surface of a cell, to thereby suppress polypeptide-of-the-invention-mediated signal transduction in vivo. The fusion proteins of immunoglobin and polypeptides of the invention can be used to affect the bioavailability of a polypeptide-of-the-invention cognate ligand. Inhibition of the polypeptide-of-the-invention ligand/polypeptide of the invention interaction may be useful therapeutically for both the treatment of proliferative and differentiative disorders, as well as for modulating (e.g. promoting or inhibiting) cell survival. Moreover, the fusion proteins of immunoglobin and polypeptides of the invention that are part of the invention can be used as immunogens to produce anti-polypepetide-of-the-invention antibodies in a subject, to purify ligands of polypeptides of the invention and in screening assays to identify molecules which inhibit the interaction of polypeptides of the invention with a ligand of a polypeptide of an invention.

Preferably, a chimeric or fusion polypeptide of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al. eds., John Wiley & Sons: 1992). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

The present invention also pertains to variants of the polypeptides of the invention (i.e., proteins having a sequence which differs from that of the amino acid sequences of polypeptides of the invention). Such variants can function as either agonists (mimetics) to polypeptides of the invention or or as antagonists of polypeptides of the invention. Variants of the polypeptides of the invention can be generated by mutagenesis, e.g., discrete point mutation or truncation of the polypeptide of the invention. An agonist of the polypeptide of the invention can retain substantially the same, or a subset, of the biological activities of the naturally occurring form of the polypeptide of the invention. An antagonist of the polypeptide of the invention can inhibit one or more of the activities of the naturally occurring form of the polypeptide of the invention by, for example, competitively binding to a downstream or upstream member of a cellular signaling cascade which includes the polypeptide of the invention. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the polypeptides of the invention.

Variants of the polypeptides of the invention that function as either agonists (mimetics) of polypeptides of the invention or as antagonists of polypeptides of the invention can be identified by screening combinatorial libraries of mutants, e.g., truncation mutants, of the polypeptides of the invention for agonist or antagonist activity with respect to polypeptides of the invention. In one embodiment, a variegated library of variants of nucleic acids and polypeptides of the invention is generated by combinatorial mutagenesis at the nucleic acid level and is encoded by a variegated gene library. A variegated library of variants of nucleic acids and polypeptides of the invention can be produced by, for example, enzymatically ligating a mixture of synthetic oligonucleotides into gene sequences such that a degenerate set of potential sequences of nucleic acids of the invention is expressible as individual polypeptides, or alternatively, as a set of larger fusion proteins (e.g., for phage display) containing the set of sequences of polypeptides of the invention therein. There are a variety of methods that can be used to produce libraries of potential variants of nucleic acids and polypeptides of the invention from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential sequences of nucleic acids and polypeptides of the invention. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of the coding sequences of nucleic acids of the invention can be used to generate a variegated population of fragments of polypeptides of the invention for screening and subsequent selection of variants of polypeptides of the invention. In one embodiment, a library of coding sequence fragments can be generated by treating a double stranded PCR fragment of a coding sequence of a nucleic acid of the invention with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the polypeptide of the invention.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of nucleic acids of the invention. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of nucleic acids and polypeptides of the invention (Arkin and Yourvan (1992) Proc. Natl. Acad. Sci. USA 89:7811-7815; Delgrave et al. (1993) Protein Engineering 6(3):327-331).

An isolated polypeptide of the invention, or a portion or fragment thereof, can be used as an immunogen to generate antibodies that bind a polypeptide of the invention using standard techniques for polyclonal and monoclonal antibody preparation. The full-length polypeptide of the invention can be used or, alternatively, the invention provides antigenic peptide fragments of polypeptides of the invention for use as immunogens. The antigenic peptide of a polypeptide of the invention comprises at least 8 (preferably 10, 15, 20, or 30) amino acid residues of the amino acid sequence of a polypeptide of the invention (e.g., that shown in SEQ ID NO:54) and encompasses an epitope of a polypeptide of the invention such that an antibody raised against the peptide forms a specific immune complex with a polypeptide of the invention.

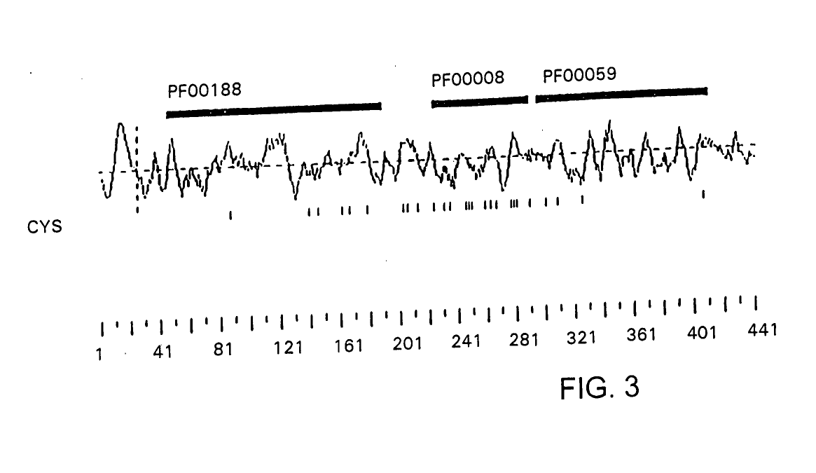

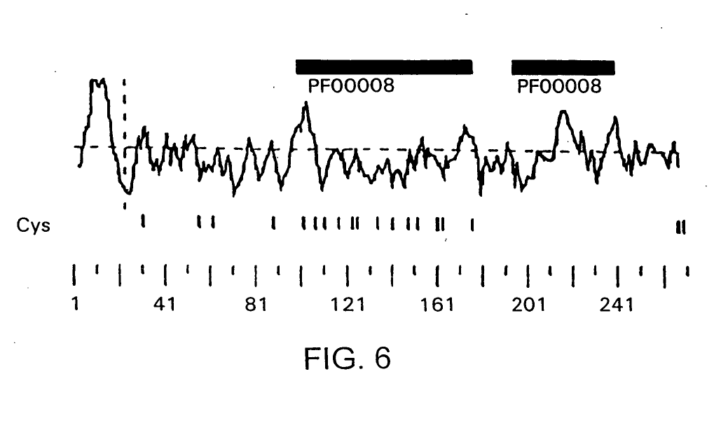

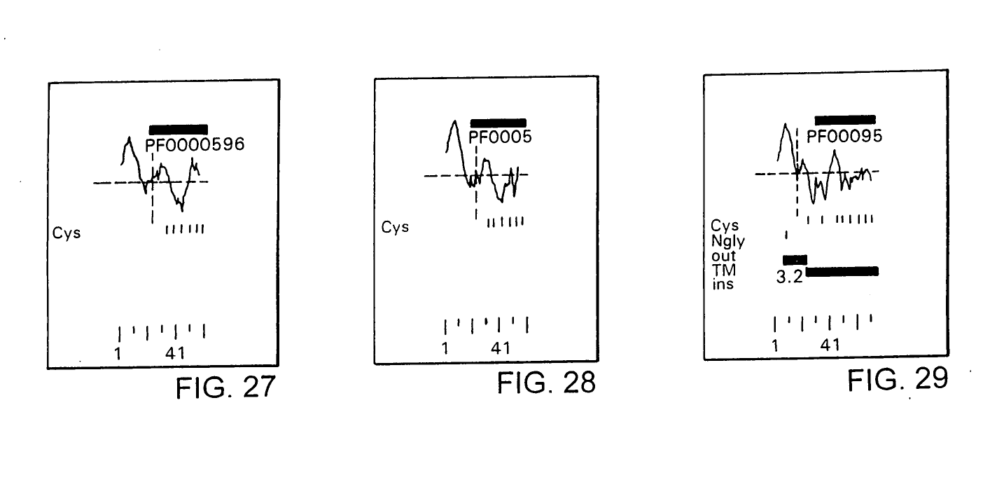

Preferred epitopes encompassed by the antigenic peptide are regions of polypeptides of the invention that are located on the surface of the protein, e.g., hydrophilic regions, and lack cysteines of n-glycosylation sites. FIGS. 3, 6, 12, 14, 17, 19, 27, 28, and 29, and are hydropathy plots of polypeptides of the invention. Hydropathy analysis, or similar analyses, can be used to identify hydrophilic regions of polypeptides of the invention.

A polypeptide-of-the-invention immunogen typically is used to prepare antibodies by immunizing a suitable subject, (e.g., rabbit, goat, mouse or other mammal) with the immunogen. An appropriate immunogenic preparation can contain, for example, recombinantly expressed polypeptide of the invention or a chemically synthesized polypeptide of the invention. The preparation can further include an adjuvant, such as Freund's complete or incomplete adjuvant, or similar immunostimulatory agent. Immunization of a suitable subject with an immunogenic polypeptide-of-the-invention preparation induces a polyclonal anti-polypeptide-of-the-invention antibody response.

Accordingly, another aspect of the invention pertains to anti-polypeptide-of-the-invention antibodies. The term “antibody” as used herein refers to immunoglobulin molecules and immunologically active portions of immunoglobulin molecules, i.e., molecules that contain an antigen binding site which specifically binds an antigen, such as a polypeptide of the invention. A molecule which specifically binds to a polypeptide of the invention is a molecule which binds a polypeptide of the invention, but does not substantially bind other molecules in a sample, e.g., a biological sample, which naturally contains a polypeptide of the invention. Examples of immunologically active portions of immunoglobulin molecules include F(ab) and F(ab)₂ fragments which can be generated by treating the antibody with an enzyme such as pepsin or papein, especially. The invention provides polyclonal and monoclonal antibodies that bind a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal anti-polypeptide-of-the-invention antibodies can be prepared as described above by immunizing a suitable subject with a polypeptide-of-the-invention immunogen. The anti-polypeptide-of-the-invention antibody titer in the immunized subject can be monitored over time by standard techniques, such as with an enzyme linked immunosorbent assay (ELISA) using immobilized polypeptide of the invention. If desired, the antibody molecules directed against a polypeptide of the invention can be isolated from the mammal (e.g., from the blood) and further purified by well-known techniques, such as protein A chromatography to obtain the IgG fraction. At an appropriate time after immunization, e.g., when the anti-polypeptide-of-the-invention antibody titers are highest, antibody-producing cells can be obtained from the subject and used to prepare monoclonal antibodies by standard techniques, such as the hybridoma technique originally described by Kohler and Milstein (1975) Nature 256:495497, the human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing various antibodies, monoclonal antibody hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with a polypeptide-of-the-invention immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating an anti-polypeptide-of-the-invention monoclonal antibody (see, e.g., Current Protocols in Immunology, supra; Galfre et al. (1977) Nature 266:55052; R. H. Kenneth, in Monoclonal Antibodies: A New Dimension In Biological Analyses, Plenum Publishing Corp., New York, N.Y. (1980); and Lemer (1981) Yale J. Biol. Med., 54:387402. Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line, e.g., a myeloma cell line that is sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a polypeptide of the invention, e.g., using a standard ELISA assay.

Alternative to preparing monoclonal antibody-secreting hybridomas, a monoclonal anti-polypeptide-of-the-invention antibody can be identified and isolated by screening a recombinant combinatorial immunoglobulin library (e.g., an antibody phage display library) with a polypeptide of the invention to thereby isolate immunoglobulin library members that bind a polypeptide of the invention. Kits for generating and screening phage display libraries are commercially available (e.g., the Pharmacia Recombinant Phage Antibody System, Catalog No. 27-9400-01; and the Stratagene SurjZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant anti-polypeptide-of-the-invention antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope.

First, a non-human monoclonal antibody which binds a selected antigen (epitope), e.g., an antibody which inhibits activity, is identified. The heavy chain and the light chain of the non-human antibody are cloned and used to create phage display Fab fragments. For example, the heavy chain gene can be cloned into a plasmid vector so that the heavy chain can be secreted from bacteria. The light chain gene can be cloned into a phage coat protein gene so that the light chain can be expressed on the surface of phage. A repertoire (random collection) of human light chains fused to phage is used to infect the bacteria which express the non-human heavy chain. The resulting progeny phage display hybrid antibodies (human light chain/non-human heavy chain). The selected antigen is used in a panning screen to select phage which bind the selected antigen. Several rounds of selection may be required to identify such phage. Next, human light chain genes are isolated from the selected phage which bind the selected antigen. These selected human light chain genes are then used to guide the selection of human heavy chain genes as follows. The selected human light chain genes are inserted into vectors for expression by bacteria. Bacteria expressing the selected human light chains are infected with a repertoire of human heavy chains fused to phage. The resulting progeny phage display human antibodies (human light chain/human heavy chain).

Next, the selected antigen is used in a panning screen to select phage which bind the selected antigen. The phage selected in this step display a completely human antibody which recognizes the same epitope recognized by the original selected, non-human monoclonal antibody. The genes encoding both the heavy and light chains are readily isolated and can be further manipulated for production of human antibody. This technology is described by Jespers et al. (1994, Bio/technology 12:899-903).

An anti-polypeptide-of-the-invention antibody (e.g., monoclonal antibody) can be used to isolate a polypeptide of the invention by standard techniques, such as affinity chromatography or immunoprecipitation. An anti-polypeptide-of-the-invention antibody can facilitate the purification of natural polypeptide of the invention from cells and of recombinantly produced polypeptide of the invention expressed in host cells. Moreover, an anti-polypeptide-of-the-invention antibody can be used to detect polypeptide of the invention (e.g., in a cellular lysate or cell supernatant) in order to evaluate the abundance and pattern of expression of the polypeptide of the invention. Anti-polypeptide-of-the-invention antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

An antibody (or fragment thereof) can be conjugated to a therapeutic moiety such as a cytotoxin, a therapeutic agent, or a radioactive agent (e.g., a radioactive metal ion). Cytotoxins and cytotoxic agents include any agent that is detrimental to cells. Examples of such agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, and 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin {formerly designated daunomycin} and doxorubicin), antibiotics (e.g., dactinomycin {formerly designated actinomycin}, bleomycin, mithramycin, and anthramycin), and anti-mitotic agents (e.g., vincristine and vinblastine).

Conjugated antibodies of the invention can be used for modifying a given biological response, the drug moiety not being limited to classical chemical therapeutic agents. For example, the drug moiety can be a protein or polypeptide possessing a desired biological activity. Such proteins include, for example, toxins such as abrin, ricin A, Pseudomonas exotoxin, or diphtheria toxin; proteins such as tumor necrosis factor, alpha-interferon, beta-interferon, nerve growth factor, platelet derived growth factor, tissue plasminogen activator; and biological response modifiers such as lymphokines, interleukin-1, interleukin-2, interleukin-6, granulocyte macrophage colony stimulating factor, granulocyte colony stimulating factor, or other growth factors.

Techniques for conjugating a therapeutic moiety to an antibody are well known (see, e.g., Arnon et al., 1985, “Monoclonal Antibodies For Immunotargeting Of Drugs In Cancer Therapy”, in Monoclonal Antibodies And Cancer Therapy, Reisfeld et al., Eds., Alan R. Liss, Inc. pp. 243-256; Hellstrom et al., 1987, “Antibodies For Drug Delivery”, in Controlled Drug Delivery, 2nd ed., Robinson et al., Eds., Marcel Dekker, Inc., pp. 623-653; Thorpe, 1985, “Antibody Carriers Of Cytotoxic Agents In Cancer Therapy: A Review”, in Monoclonal Antibodies'84: Biological And Clinical Applications, Pinchera et al., Eds., pp. 475-506; “Analysis, Results, And Future Prospective Of The Therapeutic Use Of Radiolabeled Antibody In Cancer Therapy”, in Monoclonal Antibodies For Cancer Detection And Therapy, Baldwin et al., Eds., Academic Press, pp. 303-316, 1985; and Thorpe et al., 1982, Immunol. Rev., 62:119-158). Alternatively, an antibody can be conjugated to a second antibody to form an antibody heteroconjugate as described by Segal in U.S. Pat. No. 4,676,980.

Recombinant Expression Vectors and Host Cells

Another aspect of the invention pertains to vectors, preferably expression vectors, containing a nucleic acid encoding polypeptide of the invention (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., polypeptides of the invention, mutant forms of polypeptide of the invention, fusion proteins, etc.).

The recombinant expression vectors of the invention can be designed for expression of nucleic acid or polypeptide of the invention in prokaryotic or eukaryotic cells, e.g., bacterial cells such as E. coli, insect cells (using baculovirus expression vectors), yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:3140), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the expression vector of a nucleic acid of the invention is a yeast expression vector. Examples of vectors for expression in yeast S. cerivisae include pYepSec1 (Baldari et al. (1987) EMBO J. 6:229-234), pMFa (Kurjan and Herskowitz, (1982) Cell 30:933-943), pJRY88 (Schultz et al. (1987) Gene 54:113-123), pYES2 (Invitrogen Corporation, San Diego, Calif.), and pPicZ (InVitrogen Corp, San Diego, Calif.).

Alternatively, nucleic acids or polypeptides of the invention can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid or polypeptide of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al. (supra).

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to a nucleic acid of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, nucleic acids or polypeptides of the invention can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding polypeptide of the invention or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) polypeptide of the invention. Accordingly, the invention further provides methods for producing polypeptide of the invention using the host cells of the invention. In one embodiment, the method comprises culturing the host cell of invention (into which a recombinant expression vector encoding polypeptide of the invention has been introduced) in a suitable medium such that polypeptide of the invention is produced. In another embodiment, the method further comprises isolating polypeptide of the invention from the medium or the host cell.

The host cells of the invention can also be used to produce nonhuman transgenic animals. For example, in one embodiment, a host cell of the invention is a fertilized oocyte or an embryonic stem cell into which sequences coding for polypeptide of the invention have been introduced. Such host cells can then be used to create non-human transgenic animals in which exogenous sequences of nucleic acid of the invention have been introduced into their genome or: homologous recombinant animals in which endogenous sequences of nucleic acid of the invention have been altered. Such animals are useful for studying the function and/or activity of nucleic acid of the invention and for identifying and/or evaluating modulators of activity of nucleic acid of the invention. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene of the invention has been altered by homologous recombination between the endogenous gene and an exogenous DNA molecule introduced into a cell of the animal, e.g., an embryonic cell of the animal, prior to development of the animal.

A transgenic animal of the invention can be created by introducing nucleic acid encoding polypeptide of the invention into the male pronuclei of a fertilized oocyte, e.g., by microinjection, retroviral infection, and allowing the oocyte to develop in a pseudopregnant female foster animal. The cDNA sequence of the invention, e.g., that of SEQ ID NO:1, SEQ ID NO:3, SEQ ID NO:9, SEQ ID NO:11, SEQ ID NO:29, SEQ ID NO:31, SEQ ID NO:40, SEQ ID NO:46, SEQ ID NO:47, SEQ ID NO:48, SEQ ID NO:49, SEQ ID NO:50, SEQ ID NO:51, SEQ ID NO:52, or SEQ ID NO:53 can be introduced as a transgene into the genome of a non-human animal. Alternatively, a nonhuman homologue of the human gene of the invention, can be isolated based on hybridization to the human cDNA of the invention and used as a transgene. Intronic sequences and polyadenylation signals can also be included in the transgene to increase the efficiency of expression of the transgene. A tissue-specific regulatory sequence(s) can be operably linked to the transgene of a nucleic acid of the invention to direct expression of polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene of a nucleic acid of the invention in its genome and/or expression of mRNA of the invention in tissues or cells of the animals. A transgenic founder animal can then be used to breed additional animals carrying the transgene. Moreover, transgenic animals carrying a transgene encoding polypeptide of the invention can further be bred to other transgenic animals carrying other transgenes.

To create an homologous recombinant animal, a vector is prepared which contains at least a portion of a gene of the invention (e.g., a human or a non-human homolog of the gene of the invention, e.g., a murine gene of the invention) into which a deletion, addition or substitution has been introduced to thereby alter, e.g., functionally disrupt, the gene of the invention. In a preferred embodiment, the vector is designed such that, upon homologous recombination, the endogenous gene of the invention is functionally disrupted (i.e., no longer encodes a functional protein; also referred to as a “knock out” vector). Alternatively, the vector can be designed such that, upon homologous recombination, the endogenous gene of the invention is mutated or otherwise altered but still encodes functional protein (e.g., the upstream regulatory region can be altered to thereby alter the expression of the endogenous polypeptide of the invention). In the homologous recombination vector, the altered portion of the gene of the invention is flanked at its 5′ and 3′ ends by additional nucleic acid of the gene of the invention to allow for homologous recombination to occur between the exogenous gene of the invention carried by the vector and an endogenous gene of the invention in an embryonic stem cell. The additional flanking nucleic acid of the invention is of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced gene of the invention has homologously recombined with the endogenous gene of the invention are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

Pharmaceutical Compositions

The nucleic acids of the invention, polypeptides of the invention, and anti-polypeptide-of-the-invention antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid of the invention. Such methods comprise formulating a pharmaceutically acceptable carrier with an agent which modulates expression or activity of a polypeptide or nucleic acid of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid of the invention and one or more additional active compounds.

The agent which modulates expression or activity can, for example, be a small molecule. For example, such small molecules include peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents and protein or polypeptide agents depends upon a number of factors within the ken of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid or polypeptide of the invention. Examples of doses of a small molecule include milligram or microgram amounts per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). Examples of doses of a protein or polypeptide include gram, milligram or microgram amounts per kilogram of subject or sample weight (e.g., about 1 microgram per kilogram to about 5 grams per kilogram, about 100 micrograms per kilogram to about 500 milligrams per kilogram, or about 1 milligram per kilogram to about 50 milligrams per kilogram). For antibodies, examples of dosages are from about 0.1 milligram per kilogram to 100 milligrams per kilogram of body weight (generally 10 milligrams per kilogram to 20 milligrams per kilogram). If the antibody is to act in the brain, a dosage of 50 milligrams per kilogram to 100 milligrams per kilogram is usually appropriate. It is furthermore understood that appropriate doses of one of these agents depend upon the potency of the agent with respect to the expression or activity to be modulated. Such appropriate doses can be determined using the assays described herein. When one or more of these agents is to be administered to an animal (e.g., a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide of the invention or anti-polypeptide-of-the-invention antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

It is recognized that the pharmaceutical compositions and methods described herein can be used independently or in combination with one another. That is, subjects can be administered one or more of the pharmaceutical compositions, e.g., pharmaceutical compositions comprising a nucleic acid molecule or protein of the invention or a modulator thereof, subjected to one or more of the therapeutic methods described herein, or both, in temporally overlapping or non-overlapping regimens. When therapies overlap temporally, the therapies may generally occur in any order and can be simultaneous (e.g., administered simultaneously together in a composite composition or simultaneously but as separate compositions) or interspersed. By way of example, a subject afflicted with a disorder described herein can be simultaneously or sequentially administered both a cytotoxic agent which selectively kills aberrant cells and an antibody (e.g., an antibody of the invention) which can, in one embodiment, be conjugated or linked with a therapeutic agent, a cytotoxic agent, an imaging agent, or the like.

The pharmaceutical compositions can be included in a container, pack, or dispenser together with instructions for administration.

Uses and Methods of the Invention

The nucleic acid molecules, proteins, protein homologues, and antibodies described herein can be used in one or more of the following methods: a) screening assays; b) detection assays (e.g., chromosomal mapping, tissue typing, forensic biology); c) predictive medicine (e.g., diagnostic assays, prognostic assays, monitoring clinical trials, and pharmacogenomics); and d) methods of treatment (e.g., therapeutic and prophylactic). The isolated nucleic acid molecules of the invention can be used to express polypeptide of the invention (e.g., via a recombinant expression vector in a host cell in gene therapy applications), to detect mRNA of the invention (e.g., in a biological sample) or a genetic lesion in a gene of the invention, and to modulate activity of a nucleic acid of the invention. In addition, the polypeptides of the invention can be used to screen drugs or compounds which modulate the activity or expression of nucleic acids or polypeptides of the invention as well as to treat disorders characterized by insufficient or excessive production of polypeptide of the invention or production of forms of polypeptide of the invention which have decreased or aberrant activity compared to wild type polypeptide of the invention. In addition, the anti-polypeptide-of-the-invention antibodies of the invention can be used to detect and isolate polypeptides of the invention and modulate activity of polypeptides of the invention.

This invention further pertains to novel agents identified by the above-described screening assays and uses thereof for treatments as described herein.

Screening Assays

The invention provides a method (also referred to herein as a “screening assay”) for identifying modulators, i.e., candidate or test compounds or agents (e.g., peptides, peptidomimetics, small molecules or other drugs) which bind to polypeptides of the invention or have a stimulatory or inhibitory effect on, for example, TANGO-139, 125, 110, 175, or WDNM-2 expression or TANGO-139, 125, 110, 175, or WDNM-2 activity.

In one embodiment, the invention provides assays for screening candidate or test compounds which bind with or modulate the activity of the membrane-bound form of a polypeptide of the invention or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer, or small molecule libraries of compounds (Lam (1997) Anticancer Drug Des. 12:145).

Examples of methods useful for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds can be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (Patent numbers 5,571,698; 5,403,484; and 5,223,409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

In one embodiment, an assay is a cell-based assay in which a cell which expresses a membrane-bound form of a polypeptide of the invention, or a biologically active portion thereof, on the cell surface is contacted with a test compound and the ability of the test compound to bind with the polypeptide is determined. The cell, for example, can be a yeast cell or a cell of mammalian origin. Determining the ability of the test compound to bind with the polypeptide can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radio-emission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of a polypeptide of the invention, or a biologically active portion thereof, on the cell surface with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind with the polypeptide or a biologically active portion thereof as compared to the known compound.

In another embodiment, the assay involves assessment of an activity characteristic of the polypeptide, wherein binding of the test compound with the polypeptide or a biologically active portion thereof alters (i.e., increases or decreases) the activity of the polypeptide.

In one embodiment, an assay of the present invention is a cell-free assay comprising contacting a polypeptide of the invention or biologically active portion thereof with a test compound and determining the ability of the test compound to bind to the polypeptide of the invention or biologically active portion thereof. Binding of the test compound to the polypeptide of the invention can be determined either directly or indirectly as described above. In a preferred embodiment, the assay includes contacting the polypeptide of the invention or biologically active portion thereof with a known compound which binds polypeptide of the invention to form an assay mixture, contacting the assay mixture with a test compound, and determining the ability of the test compound to interact with a polypeptide of the invention, wherein determining the ability of the test compound to interact with a polypeptide of the invention comprises determining the ability of the test compound to preferentially bind to polypeptide of the invention or biologically active portion thereof as compared to the known compound.

In another embodiment, an assay is a cell-free assay comprising contacting polypeptide of the invention or biologically active portion thereof with a test compound and determining the ability of the test compound to modulate (e.g., stimulate or inhibit) the activity of the polypeptide of the invention or biologically active portion thereof. Determining the ability of the test compound to modulate the activity of polypeptide of the invention can be accomplished, for example, by determining the ability of the polypeptide of the invention to bind to a target molecule of the polypeptide of the invention by one of the methods described above for determining direct binding. In an alternative embodiment, determining the ability of the test compound to modulate the activity of polypeptide of the invention can be accomplished by determining the ability of the polypeptide of the invention to further modulate a target molecule of the polypeptide of the invention. For example, the catalytic/enzymatic activity of the target molecule on an appropriate substrate can be determined as previously described.

In yet another embodiment, the cell-free assay comprises contacting the polypeptide of the invention or biologically active portion thereof with a known compound which binds polypeptide of the invention to form an assay mixture, contacting the assay mixture with a test compound, and determining the ability of the test compound to interact with a polypeptide of the invention, wherein determining the ability of the test compound to interact with a polypeptide of the invention comprises determining the ability of the polypeptide of the invention to preferentially bind to or modulate the activity of a target molecule of the polypeptide of the invention.

In more than one embodiment of the above assay methods of the present invention, it may be desirable to immobilize either the polypeptide of the invention or its target molecule to facilitate separation of complexed from uncomplexed forms of one or both of the proteins, as well as to accommodate automation of the assay. Binding of a test compound to polypeptide of the invention, or interaction of polypeptide of the invention with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/polypeptide-of-the-invention fusion proteins or glutathione-S-transferase/target fusion proteins can be adsorbed onto glutathione sepharose beads (Sigma Chemical; St. Louis, Mo.) or glutathione derivatized microtitre plates, which are then combined with the test compound or the test compound and either the non-adsorbed target protein or polypeptide of the invention, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of binding or activity of polypeptide of the invention determined using standard techniques.

Other techniques for immobilizing proteins on matrices can also be used in the screening assays of the invention. For example, either polypeptide of the invention or its target molecule can be immobilized utilizing conjugation of biotin and streptavidin. Biotinylated polypeptide of the invention or target molecules can be prepared from biotin-NHS(N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with polypeptide of the invention or target molecules but which do not interfere with binding of the polypeptide of the invention to its target molecule can be derivatized to the wells of the plate, and unbound target or polypeptide of the invention trapped in the wells by antibody conjugation. Methods for detecting such complexes, in addition to those described above for the GST-immobilized complexes, include immunodetection of complexes using antibodies reactive with the polypeptide of the invention or target molecule, as well as enzyme-linked assays which rely on detecting an enzymatic activity associated with the polypeptide of the invention or target molecule.

In general, determining the ability of the test compound to modulate the activity of a polypeptide of the invention or a biologically active portion thereof can be accomplished, for example, by determining the ability of the polypeptide of the invention to bind to or interact with a target molecule of the polypeptide of the invention. As used herein, a “target molecule” is a molecule with which a polypeptide of the invention binds or interacts in nature, for example, a molecule on the surface of a cell, e.g., an integrin or a extracellular. A target molecule of a polypeptide of the invention can be a non-polypeptide-of-the-invention molecule or a polypeptide of the present invention. The target, for example, can be a extracellular protein which has catalytic activity e.g., a proteinase particularly a serine proteinase.

Determining the ability of the polypeptide of the invention to bind to or interact with a target molecule of the polypeptide of the invention can be accomplished by one of the methods described above for determining direct binding. In a preferred embodiment, determining the ability of the polypeptide of the invention to bind to or interact with a target molecule of the polypeptide of the invention can be accomplished by determining the activity of the target molecule. For example, the activity of the target molecule can be determined by detecting catalytic/enzymatic activity of the target (e.g., a proteinase) on an appropriate substrate, detecting the induction of a reporter gene (e.g., a regulatory element responsive to a TANGO-139, 125, 110, 175 or WDNM-2 generated signal operatively linked to a nucleic acid encoding a detectable marker, e.g. luciferase), or detecting a cellular response.

In another embodiment, modulators of expression of nucleic acids or polypeptides of the invention are identified in a method in which a cell is contacted with a candidate compound and the expression of mRNA or polypeptide of the invention in the cell is determined. The level of expression of mRNA or polypeptide of the invention in the presence of the candidate compound is compared to the level of expression of mRNA or polypeptide of the invention in the absence of the candidate compound. The candidate compound can then be identified as a modulator of expression of mRNA or polypeptide of the invention based on this comparison. For example, when expression of mRNA or polypeptide of the invention is greater (statistically significantly greater) in the presence of the candidate compound than in its absence, the candidate compound is identified as a stimulator of expression of mRNA or polypeptide of the invention. Alternatively, when expression of mRNA or polypeptide of the invention is less (statistically significantly less) in the presence of the candidate compound than in its absence, the candidate compound is identified as an inhibitor of expression of mRNA or polypeptide of the invention. The level of expression of mRNA or polypeptide of the invention in the cells can be determined by methods described herein for detecting mRNA or polypeptide of the invention.

In yet another aspect of the invention, the polypeptides of the invention can be used as “bait proteins” in a two-hybrid assay or three hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with polypeptides of the invention (“polypeptide-of-the-invention-binding proteins” or “polypeptide-of-the-invention-bp”) and modulate activity of polypeptide of the invention. Such polypeptide-of-the-invention-binding proteins are also likely to be involved in the propagation of signals by the polypeptides of the invention as, for example, upstream or downstream elements of the TANGO-139, 125, 110, 175, or WDNM-2 pathway.

The two-hybrid system is based on the modular nature of most transcription factors, which consist of separable DNA-binding and activation domains. Briefly, the assay utilizes two different DNA constructs. In one construct, the gene that codes for a polypeptide of the invention is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a TANGO-139, 125, 110, 175, or WDNM-2-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with TANGO-139, 125, 110, 175, or WDNM-2.

This invention further pertains to novel agents identified by the above-described screening assays and uses thereof for treatments as described herein.

Detection Assays

Portions or fragments of the cDNA sequences identified herein (and the corresponding complete gene sequences) can be used in numerous ways as polynucleotide reagents. For example, these sequences can be used to: (i) map their respective genes on a chromosome and, thus, locate gene regions associated with genetic disease; (ii) identify an individual from a minute biological sample (tissue typing); and (iii) aid in forensic identification of a biological sample. These applications are described in the subsections below.

Chromosome Mapping

Once the sequence (or a portion of the sequence) of a gene has been isolated, this sequence can be used to map the location of the gene on a chromosome. Accordingly, nucleic acids of the invention described herein or fragments thereof, can be used to map the location of genes of the invention on a chromosome. The mapping of the sequences of nucleic acids of the invention to chromosomes is an important first step in correlating these sequences with genes associated with disease.

Briefly, genes of the invention can be mapped to chromosomes by preparing PCR primers (preferably 15-25 bp in length) from the sequences of nucleic acids of the invention. Computer analysis of sequences of nucleic acids of the invention can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the sequences of nucleic acids of the invention will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow (because they lack a particular enzyme), but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the sequences of nucleic acids of the invention to design oligonucleotide primers, sublocalization can be achieved with panels of fragments from specific chromosomes. Other mapping strategies which can similarly be used to map a sequence of a nucleic acid of the invention to its chromosome include in situ hybridization (described in Fan et al. (1990) Proc. Natl. Acad. Sci. USA 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical, e.g., colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., (Human Chromosomes: A Manual of Basic Techniques (Pergamon Press, New York, 1988)).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature 325:783-787.

Moreover, differences in the DNA sequences between individuals affected and unaffected with a disease associated with the genes of the invention can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The sequences of nucleic acids of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the sequences of nucleic acids of the invention described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The sequences of nucleic acids of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. For example, the noncoding sequences of SEQ ID NO:1, SEQ ID NO:9, SEQ ID NO:29, SEQ ID NO:46, SEQ ID NO:47, SEQ ID NO:48, or SEQ ID NO:49 can comfortably provide positive individual identification with a panel of perhaps 10 to 1,000 primers which each yield a noncoding amplified sequence of 100 bases. If predicted coding sequences, such as those in SEQ ID NO:3, SEQ ID NO:11, SEQ ID NO:31, SEQ ID NO:40, SEQ ID NO:50, SEQ ID NO:51, SEQ ID NO:52, or SEQ ID NO:53 are used, a more appropriate number of primers for positive individual identification would be 500-2,000.

If a panel of reagents from sequences of nucleic acids of the invention described herein is used to generate a unique identification database for an individual, those same reagents can later be used to identify tissue from that individual. Using the unique identification database, positive identification of the individual, living or dead, can be made from extremely small tissue samples.

Use of Partial TANGO-139, 125, 110, 175, or WDNM-2 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. For example, sequences targeted to noncoding regions of SEQ ID NO:1, SEQ ID NO:9, SEQ ID NO:29, SEQ ID NO:46, SEQ ID NO:47, SEQ ID NO:48, or SEQ ID NO:49 are particularly appropriate for this use as greater numbers of polymorphisms occur in the noncoding regions, making it easier to differentiate individuals using this technique. Examples of polynucleotide reagents include the sequences of nucleic acids of the invention or portions thereof, e.g., fragments derived from the noncoding regions of SEQ ID NO:1, SEQ ID NO:9, SEQ ID NO:29, SEQ ID NO:46, SEQ ID NO:47, SEQ ID NO:48, or SEQ ID NO:49 having a length of at least 20 or 30 bases.

The sequences of nucleic acids of the invention described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such nucleic-acid-of-the invention probes can be used to identify tissue by species and/or by organ type.

In a similar fashion, these reagents, e.g., nucleic-acid-of-the-invention primers or probes can be used to screen tissue culture for contamination (i.e., screen for the presence of a mixture of different types of cells in a culture).

Predictive Medicine

The present invention also pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining expression of polypeptides and/or nucleic acids of the invention as well as activity of nucleic acids or polypeptides of the invention, in the context of a biological sample (e.g., blood, serum, cells, tissue) to thereby determine whether an individual is afflicted with a disease or disorder, or is at risk of developing a disorder, associated with aberrant expression or activity of nucleic acids of polypeptides of the invention. The invention also provides for prognostic (or predictive) assays for determining whether an individual is at risk of developing a disorder associated with expression or activity of nucleic acids or polypeptides of the invention. For example, mutations in a gene of the invention can be assayed in a biological sample. Such assays can be used for prognostic or predictive purpose to thereby prophylactically treat an individual prior to the onset of a disorder characterized by or associated with expression or activity or nucleic acids or polypeptides of the invention.

As an alternative to making determinations based on the absolute expression level of selected genes, determinations may be based on the normalized expression levels of these genes. Expression levels are normalized by correcting the absolute expression level of a gene encoding a polypeptide of the invention by comparing its expression to the expression of a different gene, e.g., a housekeeping gene that is constitutively expressed. Suitable genes for normalization include housekeeping genes such as the actin gene. This normalization allows the comparison of the expression level in one sample (e.g., a patient sample), to another sample, or between samples from different sources.

Alternatively, the expression level can be provided as a relative expression level. To determine a relative expression level of a gene, the level of expression of the gene is determined for 10 or more samples of different endothelial (e.g. intestinal endothelium, airway endothelium, or other mucosal epithelium) cell isolates, preferably 50 or more samples, prior to the determination of the expression level for the sample in question. The mean expression level of each of the genes assayed in the larger number of samples is determined and this is used as a baseline expression level for the gene(s) in question. The expression level of the gene determined for the test sample (absolute level of expression) is then divided by the mean expression value obtained for that gene. This provides a relative expression level and aids in identifying extreme cases of disorders associated with aberrant expression of a gene encoding a polypeptide of the invention protein or with aberrant expression of a ligand thereof.

Preferably, the samples used in the baseline determination will be from either or both of cells which aberrantly express a gene encoding a polypeptide of the invention or a ligand thereof (i.e. ‘diseased cells’) and cells which express a gene encoding a polypeptide of the invention at a normal levelor a ligand thereof (i.e. ‘normal’ cells). The choice of the cell source is dependent on the use of the relative expression level. Using expression found in normal tissues as a mean expression score aids in validating whether aberrance in expression of a gene encoding a polypeptide of the invention occurs specifically in diseased cells. Such a use is particularly important in identifying whether a gene encoding a polypeptide of the invention can serve as a target gene. In addition, as more data is accumulated, the mean expression value can be revised, providing improved relative expression values based on accumulated data. Expression data from endothelial cells (e.g. mucosal endothelial cells) provides a means for grading the severity of the disorder.

Another aspect of the invention provides methods for determining expression or activity of nucleic acids or polypeptides of the invention in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs or other compounds) on the expression or activity of nucleic acids or polypeptides of the invention in clinical trials.

These and other agents are described in further detail in the following sections.

Diagnostic Assays

An exemplary method for detecting the presence or absence of nucleic acids or polypeptide of the invention in a biological sample involves obtaining a biological sample from a test subject and contacting the biological sample with a compound or an agent capable of detecting polypeptide of the invention or nucleic acid (e.g., mRNA, genomic DNA) that encodes polypeptide of the invention such that the presence of polypeptide or nucleic acids of the invention is detected in the biological sample. A preferred agent for detecting mRNA or genomic DNA of the invention is a labeled nucleic acid probe capable of hybridizing to mRNA or genomic DNA of the invention. The nucleic acid probe can be, for example, a full-length nucleic acid of the invention, such as the nucleic acid of SEQ ID NO:33, SEQ ID NO:35, SEQ ID NO:41, SEQ ID NO:43, or SEQ ID NO:45, or a portion thereof, such as an oligonucleotide of at least 15, 30, 50, 100, 250 or 400 nucleotides in length and sufficient to specifically hybridize under stringent conditions to mRNA or genomic DNA of the invention. Other suitable probes for use in the diagnostic assays of the invention are described herein.

A preferred agent for detecting polypeptide of the invention is an antibody capable of binding to polypeptide of the invention, preferably an antibody with a detectable label. Antibodies can be polyclonal, or more preferably, monoclonal. An intact antibody, or a fragment thereof (e.g., Fab or F(ab)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect mRNA, polypeptide, or genomic DNA of the invention in a biological sample in vitro as well as in vivo. For example, in vitro techniques for detection of mRNA of the invention include Northern hybridizations and in situ hybridizations. In vitro techniques for detection of polypeptide of the invention include enzyme linked immunosorbent assays (ELISAs), Western blots, immunoprecipitations and immunofluorescence. In vitro techniques for detection of genomic DNA of the invention include Southern hybridizations. Furthermore, in vivo techniques for detection of polypeptide of the invention include introducing into a subject a labeled anti-polypeptide-of-the-invention antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting polypeptide, mRNA, or genomic DNA of the invention, such that the presence of polypeptide, mRNA, or genomic DNA of the invention is detected in the biological sample, and comparing the presence of polypeptide, mRNA, or genomic DNA of the invention in the control sample with the presence of polypeptide, mRNA, or genomic DNA of the invention in the test sample.

The invention also encompasses kits for detecting the presence of nucleic acids or polypeptides of the invention in a biological sample (a test sample). Such kits can be used to determine if a subject is suffering from or is at increased risk of developing a disorder associated with aberrant expression of nucleic acids or polypeptides of the invention (e.g., an immunological disorder). For example, the kit can comprise a labeled compound or agent capable of detecting polypeptide or mRNA of the invention in a biological sample and means for determining the amount of polypeptide or mRNA of the invention in the sample (e.g., an anti-polypeptide-of-the-invention antibody or an oligonucleotide probe which binds to DNA encoding polypeptide of the invention (e.g., SEQ ID NO:1, SEQ ID NO:3, SEQ ID NO:9, SEQ ID NO:11, SEQ ID NO:29, SEQ ID NO:31, SEQ ID NO:40, SEQ ID NO:46, SEQ ID NO:47, SEQ ID NO:48, SEQ ID NO:49, SEQ ID NO:50, SEQ ID NO:51, SEQ ID NO:52, or SEQ ID NO:53). Kits may also include instruction for observing that the tested subject is suffering from or is at risk of developing a disorder associated with aberrant expression of nucleic acid or polypeptide of the invention if the amount of polypeptide of mRNA of the invention is above or below a normal level.

For antibody-based kits, the kit may comprise, for example: (1) a first antibody (e.g., attached to a solid support) which binds to polypeptide of the invention; and, optionally, (2) a second, different antibody which binds to polypeptide of the invention or the first antibody and is conjugated to a detectable agent.

For oligonucleotide-based kits, the kit may comprise, for example: (1) an oligonucleotide, e.g., a detectably labelled oligonucleotide, which hybridizes to the sequence of a nucleic acid of the invention or (2) a pair of primers useful for amplifying a nucleic acid of the invention.

The kit may also comprise, e.g., a buffering agent, a preservative, or a protein stabilizing agent. The kit may also comprise components necessary for detecting the detectable agent (e.g., an enzyme or a substrate). The kit may also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing a disorder associated with aberrant expression of nucleic acids or polypeptides of the invention.

Prognostic Assays

The methods described herein can furthermore be utilized as diagnostic or prognostic assays to identify subjects having or at risk of developing a disease or disorder associated with aberrant expression or activity of nucleic acids or polypeptides of the invention. For example, the assays described herein, such as the preceding diagnostic assays or the following assays, can be utilized to identify a subject having or at risk of developing a disorder associated with expression or activity of a nucleic acid or polypeptide of the invention. Alternatively, the prognostic assays can be utilized to identify a subject having or at risk for developing such a disease or disorder. Thus, the present invention provides a method in which a test sample is obtained from a subject and nucleic acid (e.g., mRNA, genomic DNA) or polypeptide of the invention is detected, wherein the presence of nucleic acid or polypeptide of the invention is diagnostic for a subject having or at risk of developing a disease or disorder associated with aberrant expression or activity of nucleic acids or polypeptides of the invention. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant expression or activity of nucleic acids or polypeptides of the invention. For example, such methods can be used to determine whether a subject can be effectively treated with a specific agent or class of agents (e.g., agents of a type which decrease TANGO-139, 125, 110, 175, WDNM-2 activity). Thus, the present invention provides methods for determining whether a subject can be effectively treated with an agent for a disorder associated with aberrant expression or activity of nucleic acids or polypeptides of the invention in which a test sample is obtained and nucleic acid or polypeptide of the invention is detected (e.g., wherein the presence of nucleic acid or polypeptide of the invention is diagnostic for a subject that can be administered the agent to treat a disorder associated with aberrant expression or activity of nucleic acid or polypeptide of the invention).

The methods of the invention can also be used to detect genetic lesions or mutations in a gene of the invention, thereby determining if a subject with the lesioned gene is at risk for a disorder characterized by aberrant cell proliferation and/or differentiation. In preferred embodiments, the methods include detecting, in a sample of cells from the subject, the presence or absence of a genetic lesion or mutation characterized by at least one of an alteration affecting the integrity of a gene encoding a polypeptide of the invention, or the mis-expression of the gene of the invention. For example, such genetic lesions or mutations can be detected by ascertaining the existence of at least one of: 1) a deletion of one or more nucleotides from a gene of the invention; 2) an addition of one or more nucleotides to a gene of the invention; 3) a substitution of one or more nucleotides of a gene of the invention; 4) a chromosomal rearrangement of a gene of the invention; 5) an alteration in the level of a messenger RNA transcript of a gene of the invention; 6) an aberrant modification of a gene of the invention, such as of the methylation pattern of the genomic DNA; 7) the presence of a non-wild type splicing pattern of a messenger RNA transcript of a gene of the invention; 8) a non-wild type level of a polypeptide of the invention; 9) allelic loss of a gene of the invention; and 10) an inappropriate post-translational modification of a polypeptide of the invention. As described herein, there are a large number of assay techniques known in the art which can be used for detecting lesions or mutations in a gene of the invention. A preferred biological sample is a peripheral blood leukocyte sample isolated by conventional means from a subject.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a gene of the invention (see, e.g., Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a gene of the invention under conditions such that hybridization and amplification of the gene of the invention (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh, et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a gene of the invention from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations in nucleic acids of the invention can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). For example, genetic mutations in nucleic acids of the invention can be identified in two-dimensional arrays containing light-generated DNA probes as described in Cronin et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence a gene of the invention and detect mutations by comparing the sequence of the sample gene of the invention with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a gene of the invention include methods in which protection from cleavage agents is used to detect mismatched bases in RNA/RNA or RNA/DNA heteroduplexes (Myers et al. (1985) Science 230:1242). In general, the technique of “mismatch cleavage” entails providing heteroduplexes formed by hybridizing (labeled) RNA or DNA containing the wild-type sequence of a nucleic acid of the invention with potentially mutant RNA or DNA obtained from a tissue sample. The double-stranded duplexes are treated with an agent which cleaves single-stranded regions of the duplex such as which will exist due to basepair mismatches between the control and sample strands. RNA/DNA duplexes can be treated with RNase to digest mismatched regions, and DNA/DNA hybrids can be treated with S1 nuclease to digest mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al. (1988) Proc. Natl. Acad. Sci. USA 85:4397; Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in cDNAs of the invention obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on the sequence of a nucleic acid of the invention, e.g., a wild-type sequence of a nucleic acid of the invention, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in genes of the invention. For example, single strand conformation polymorphism (SSCP) may be used to detect differences in electrophoretic mobility between mutant and wild type nucleic acids (Orita et al. (1989) Proc. Natl. Acad. Sci. USA 86:2766; see also Cotton (1993) Mutat. Res. 285:125-144; Hayashi (1992) Genet. Anal. Tech. Appl. 9:73-79). Single-stranded DNA fragments of sample and control nucleic acids of the invention will be denatured and allowed to renature. The secondary structure of single-stranded nucleic acids varies according to sequence, and the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a gene of the invention.

Furthermore, any cell type or tissue, preferably peripheral blood leukocytes, in which nucleic acid or polypeptide of the invention is expressed may be utilized in the prognostic assays described herein.

Pharmacogenomics

Agents, or modulators which have a stimulatory or inhibitory effect on activity of nucleic acids or polypeptides of the invention (e.g., gene expression of nucleic acids of the invention) as identified by a screening assay described herein can be administered to individuals to treat (prophylactically or therapeutically) disorders (e.g., proliferative) associated with aberrant activity of nucleic acids or polypeptides of the invention. In conjunction with such treatment, the pharinacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of polypeptide of the invention, expression of nucleic acid of the invention, or mutation content of genes of the invention in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.”. Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM shows no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of polypeptides of the invention, expression of nucleic acids of the invention, or mutation content of genes of the invention in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of a nucleic acid or polypeptide of the invention, such as a modulator identified by one of the exemplary screening assays described herein.

Monitoring of Effects During Clinical Trials

Monitoring the influence of agents (e.g., drugs, compounds) on the expression or activity of nucleic acids or polypeptides of the invention (e.g., the ability to modulate aberrant cell proliferation and/or differentiation) can be applied not only in basic drug screening, but also in clinical trials. For example, the effectiveness of an agent, as determined by a screening assay as described herein, to increase gene expression, protein levels, or protein activity of nucleic acids or polypeptides of the invention, can be monitored in clinical trials of subjects exhibiting decreased gene expression, protein levels, or protein activity of nucleic acids or polypeptides of the invention. Alternatively, the effectiveness of an agent, as determined by a screening assay, to decrease gene expression, protein levels, or protein activity of nucleic acids or polypeptides of the invention, can be monitored in clinical trials of subjects exhibiting increased gene expression, protein levels, or protein activity of nucleic acids or polypeptides of the invention. In such clinical trials, the expression or activity of genes of the invention and, preferably, other genes that have been implicated in, for example, a cellular proliferation disorder can be used as a marker of the immune responsiveness of a particular cell.

For example, and not by way of limitation, genes, including genes of the invention, that are modulated in cells by treatment with an agent (e.g., compound, drug or small molecule) which modulates activity of genes of the invention (e.g., as identified in a screening assay described herein) can be identified. Thus, to study the effect of agents on cellular proliferation disorders, for example, in a clinical trial, cells can be isolated and RNA prepared and analyzed for the levels of expression of genes of the invention and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of genes of the invention or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of a nucleic acid (including mRNA or genomic DNA) or polypeptide of the invention in the preadministration sample; (iii) obtaining one or more post-administration samples from the subject; (iv) detecting the level of expression or activity of the nucleic acid (including mRNA or genomic DNA) or polypeptide of the invention in the post-administration samples; (v) comparing the level of expression or activity of the nucleic acid (including mRNA or genomic DNA) or polypeptide of the invention in the pre-administration sample with the nucleic acid (including mRNA or genomic DNA) or polypeptide of the invention in the post administration sample or samples; and (vi) altering the administration of the agent to the subject accordingly. For example, increased administration of the agent may be desirable to increase the expression or activity of a nucleic acid or polypeptide of the invention to higher levels than detected, i.e., to increase the effectiveness of the agent. Alternatively, decreased administration of the agent may be desirable to decrease expression or activity of a nucleic acid or polypeptide of the invention to lower levels than detected, i.e., to decrease the effectiveness of the agent.

Methods of Treatment

The present invention provides for both prophylactic and therapeutic methods of treating a subject at risk of (or susceptible to) a disorder or having a disorder associated with aberrant expression or activity of a nucleic acid or polypeptide of the invention. Such disorders include, but are by no means limited to, the following illustrative examples:
TANGO-139: e.g., kidney defects such as kidney failure or hyperplasia.
TANGO-125: e.g., wound healing and cancer.
TANGO-110: e.g., neoplasia, inappropriate angiogenesis, or inappropriate tissue regeneration.
TANGO-175 or WDNM-2: e.g., cancer, inflammatory disorders, and hematopoietic disorders.

Further examples of disorders are provided below.

Prophylactic Methods

In one aspect, the invention provides a method for preventing in a subject, a disease or condition associated with an aberrant expression or activity of a nucleic acid or polypeptide of the invention, by administering to the subject an agent which modulates expression of a nucleic acid or polypeptide of the invention or at least one activity of a nucleic acid or polypeptide of the invention. Subjects at risk for a disease which is caused or contributed to by aberrant expression or activity of a nucleic acid or polypeptide of the invention can be identified by, for example, any or a combination of diagnostic or prognostic assays as described herein. Administration of a prophylactic agent can occur prior to the manifestation of symptoms characteristic of the aberrancy of a nucleic acid or polypeptide of the invention, such that a disease or disorder is prevented or, alternatively, delayed in its progression. Depending on the type of aberrancy of a nucleic acid or polypeptide of the invention, for example, an agonist of a polypeptide of the invention or an antagonist agent of a polypeptide of the invention can be used for treating the subject. The appropriate agent can be determined based on screening assays described herein.

Therapeutic Methods

Another aspect of the invention pertains to methods of modulating expression or activity of a nucleic acid or polypeptide for therapeutic purposes. The modulatory method of the invention involves contacting a cell with an agent that modulates one or more of the activities of the activity of a polypeptide of the invention associated with the cell. An agent that modulates activity of a polypeptide of the invention can be an agent as described herein, such as a nucleic acid or a protein, a naturally-occurring cognate ligand of a polypeptide of the invention, a peptide, a peptidomimetic of a polypeptide of the invention, or other small molecule. In one embodiment, the agent stimulates one or more of the biological activities of a polypeptide of the invention. Examples of such stimulatory agents include active polypeptides of the invention and a nucleic acid molecule encoding a polypeptide of the invention that has been introduced into the cell. In another embodiment, the agent inhibits one or more of the biological activities of a polypeptide of the invention. Examples of such inhibitory agents include antisense molecules of nucleic acids of the invention and anti-polypeptide-of-the-invention antibodies. These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant expression or activity of a nucleic acid or polypeptide molecule of the invention molecule. In one embodiment, the method involves administering an agent (e.g., an agent identified by a screening assay described herein), or combination of agents that modulates (e.g., upregulates or downregulates) expression or activity of a nucleic acid or polypeptide of the invention. In another embodiment, the method involves administering a nucleic acid or polypeptide molecule of the invention as therapy to compensate for reduced or aberrant expression or activity of a nucleic acid or polypeptide of the invention.

Stimulation of activity of a nucleic acid or polypeptide of the invention is desirable in situations in which a nucleic acid or polypeptide of the invention is abnormally downregulated and/or in which increased activity of a nucleic acid or polypeptide of the invention is likely to have a beneficial effect. Conversely, inhibition of activity of a nucleic acid or polypeptide of the invention is desirable in situations in which a nucleic acid or polypeptide of the invention is abnormally upregulated and/or in which decreased activity of a nucleic acid or polypeptide of the invention is likely to have a beneficial effect.

Disorders

Tissue Related Disorders

TANGO 139, 125, 110, 175 or WDNM-2 polypeptides, nucleic acids, and modulators thereof can be used to modulate the function, morphology, proliferation and/or differentiation of cells in the tissues in which it is expressed. Such molecules can be used to treat disorders associated with abnormal or aberrant metabolism or function of cells in the tissues in which it is expressed. Tissues in which nucleic acids and polypeptides of the invention are expressed include, for example, pancreas, kidney, testis, heart, brain, liver, placenta, lung, skeletal muscle, or small intestine.

In another example, TANGO 125 and 110 polypeptides, nucleic acids, and modulators thereof can be used to treat pancreatic disorders, such as pancreatitis (e.g., acute hemorrhagic pancreatitis and chronic pancreatitis), pancreatic cysts (e.g., congenital cysts, pseudocysts, and benign or malignant neoplastic cysts), pancreatic tumors (e.g., pancreatic carcinoma and adenoma), diabetes mellitus (e.g., insulin- and non-insulin-dependent types, impaired glucose tolerance, and gestational diabetes), or islet cell tumors (e.g., insulinomas, adenomas, Zollinger-Ellison syndrome, glucagonomas, and somatostatinoma).

As TANGO 125, 110, and 175 exhibits expression in the heart, TANGO 125, 110, and 175 nucleic acids, proteins, and modulators thereof can be used to treat heart disorders, e.g., ischemic heart disease, atherosclerosis, hypertension, angina pectoris, Hypertrophic Cardiomyopathy, and congenital heart disease.

In another example, TANGO 125 and 110 polypeptides, nucleic acids, and modulators thereof can be used to treat placental disorders, such as toxemia of pregnancy (e.g., preeclampsia and eclampsia), placentitis, or spontaneous abortion.

In another example, TANGO 125, 110, and 175 polypeptides, nucleic acids, and modulators thereof can be used to treat pulmonary (lung) disorders, such as atelectasis, cystic fibrosis, rheumatoid lung disease, pulmonary congestion or edema, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, bronchiolitis, Goodpasture's syndrome, idiopathic pulmonary fibrosis, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchiolovlveolar carcinoma, bronchial carcinoid, hamartoma, and mesenchymal tumors).

In another example, TANGO 125, 110, and 175 polypeptides, nucleic acids, and modulators thereof can be used to treat disorders of skeletal muscle, such as muscular dystrophy (e.g., Duchenne Muscular Dystrophy, Becker Muscular Dystrophy, Emery-Dreifuss Muscular Dystrophy, Limb-Girdle Muscular Dystrophy, Facioscapulohumeral Muscular Dystrophy, Myotonic Dystrophy, Oculopharyngeal Muscular Dystrophy, Distal Muscular Dystrophy, and Congenital Muscular Dystrophy), motor neuron diseases (e.g., Amyotrophic Lateral Sclerosis, Infantile Progressive Spinal Muscular Atrophy, Intermediate Spinal Muscular Atrophy, Spinal Bulbar Muscular Atrophy, and Adult Spinal Muscular Atrophy), myopathies (e.g., inflammatory myopathies (e.g., Dermatomyositis and Polymyositis), Myotonia Congenita, Paramyotonia Congenita, Central Core Disease, Nemaline Myopathy, Myotubular Myopathy, and Periodic Paralysis), and metabolic diseases of muscle (e.g., Phosphorylase Deficiency, Acid Maltase Deficiency, Phosphofructokinase Deficiency, Debrancher Enzyme Deficiency, Mitochondrial Myopathy, Carnitine Deficiency, Carnitine Palmityl Transferase Deficiency, Phosphoglycerate Kinase Deficiency, Phosphoglycerate Mutase Deficiency, Lactate Dehydrogenase Deficiency, and Myoadenylate Deaminase Deficiency).

In another example, TANGO 125, 110, and 175 polypeptides, nucleic acids, and modulators thereof can be used to treat cardiovascular disorders, such as ischemic heart disease (e.g., angina pectoris, myocardial infarction, and chronic ischemic heart disease), hypertensive heart disease, pulmonary heart disease, valvular heart disease (e.g., rheumatic fever and rheumatic heart disease, endocarditis, mitral valve prolapse, and aortic valve stenosis), congenital heart disease (e.g., valvular and vascular obstructive lesions, atrial or ventricular septal defect, and patent ductus arteriosus), or myocardial disease (e.g., myocarditis, congestive cardiomyopathy, and hypertrophic cariomyopathy).

In another example, TANGO 125, 110, and 175 polypeptides, nucleic acids, and modulators thereof can be used to treat hepatic (liver) disorders, such as jaundice, hepatic failure, hereditary hyperbiliruinemias (e.g., Gilbert's syndrome, Crigler-Naijar syndromes and Dubin-Johnson and Rotor's syndromes), hepatic circulatory disorders (e.g., hepatic vein thrombosis and portal vein obstruction and thrombosis), hepatitis (e.g., chronic active hepatitis, acute viral hepatitis, and toxic and drug-induced hepatitis), cirrhosis (e.g., alcoholic cirrhosis, biliary cirrhosis, and hemochromatosis), or malignant tumors (e.g., primary carcinoma, hepatoma, hepatoblastoma, liver cysts, and angiosarcoma).

In another example, TANGO 139, 125, 110, and 175 polypeptides, nucleic acids, and modulators thereof can be used to treat renal (kidney) disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, polycystic kidney disease, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, gout, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

In another example, TANGO 139, 125, and 175 polypeptides, nucleic acids, and modulators thereof can be used to treat testicular disorders, such as unilateral testicular enlargement (e.g., nontuberculous, granulomatous orchitis); inflammatory diseases resulting in testicular dysfunction (e.g., gonorrhea and mumps); cryptorchidism; sperm cell disorders (e.g., immotile cilia syndrome and germinal cell aplasia); acquired testicular defects (e.g., viral orchitis); and tumors (e.g., germ cell tumors, interstitial cell tumors, androblastoma, testicular lymphoma and adenomatoid tumors).

As TANGO 175 was found in a uterine smooth muscle library, TANGO 175 polypeptides, nucleic acids, and modulators thereof can be used to treat uterine disorders, e.g., hyperplasia of the endometrium, uterine cancers (e.g., uterine leiomyomoma, uterine cellular leiomyoma, leiomyosarcoma of the uterus, malignant mixed mullerian Tumor of uterus, uterine Sarcoma), and dysfunctional uterine bleeding (DUB).

In another example, TANGO 125 and 110 polypeptides, nucleic acids, and modulators thereof can be used to treat disorders of the brain, such as cerebral edema, hydrocephalus, brain herniations, iatrogenic disease (due to, e.g., infection, toxins, or drugs), inflammations (e.g., bacterial and viral meningitis, encephalitis, and cerebral toxoplasmosis), cerebrovascular diseases (e.g., hypoxia, ischemia, and infarction, intracranial hemorrhage and vascular malformations, and hypertensive encephalopathy), and tumors (e.g., neuroglial tumors, neuronal tumors, tumors of pineal cells, meningeal tumors, primary and secondary lymphomas, intracranial tumors, and medulloblastoma), and to treat injury or trauma to the brain.

As TANGO 110 was originally found in a fetal spleen library, TANGO 110 nucleic acids, proteins, and modulators thereof can be used to modulate the proliferation, differentiation, and/or function of cells that form the spleen, e.g., cells of the splenic connective tissue, e.g., splenic smooth muscle cells and/or endothelial cells of the splenic blood vessels. TANGO 110 nucleic acids, proteins, and modulators thereof can also be used to modulate the proliferation, differentiation, and/or function of cells that are processed, e.g., regenerated or phagocytized within the spleen, e.g., erythrocytes and/or B and T lymphocytes and macrophages. Thus TANGO 110 nucleic acids, proteins, and modulators thereof can be used to treat spleen, e.g., the fetal spleen, associated diseases and disorders. Examples of splenic diseases and disorders include e.g., splenic lymphoma and/or splenomegaly, and/or phagocytotic disorders, e.g., those inhibiting macrophage engulfment of bacteria and viruses in the bloodstream.

As murine TANGO-175 was originally found in a bone marrow library, TANGO-175 nucleic acids, proteins, and modulators thereof can be used to modulate the proliferation, differentiation, and/or function of cells that appear in the bone marrow, e.g., stem cells (e.g., hematopoietic stem cells), and blood cells, e.g., erythrocytes, platelets, and leukocytes. Thus TANGO-175 nucleic acids, proteins, and modulators thereof can be used to treat bone marrow, blood, and hematopoietic associated diseases and disorders, e.g., acute myeloid leukemia, hemophilia, leukemia, anemia (e.g., sickle cell anemia), and thalassemia.

In another example, TANGO 125 polypeptides, nucleic acids, and modulators thereof can be used to treat prostate disorders, such as inflammatory diseases (e.g., acute and chronic prostatitis and granulomatous prostatitis), hyperplasia (e.g., benign prostatic hypertrophy or hyperplasia), or tumors (e.g., carcinomas).

In another example, TANGO 125 polypeptides, nucleic acids, and modulators thereof can be used to treat ovarian disorders, such as ovarian endometriosis, non-neoplastic cysts (e.g., follicular and luteal cysts and polycystic ovaries) and tumors (e.g., tumors of surface epithelium, germ cell tumors, ovarian fibroma, sex cord-stromal tumors, and ovarian cancers (e.g., metastatic carcinomas, and ovarian teratoma).

In another example, TANGO 125 polypeptides, nucleic acids, and modulators thereof can be used to treat intestinal disorders, such as ischemic bowel disease, infective enterocolitis, Crohn's disease, benign tumors, malignant tumors (e.g., argentaffinomas, lymphomas, adenocarcinomas, and sarcomas), malabsorption syndromes (e.g., celiac disease, tropical sprue, Whipple's disease, and abetalipoproteinemia), obstructive lesions, hernias, intestinal adhesions, intussusception, or volvulus.

In another example, TANGO 125 polypeptides, nucleic acids, and modulators thereof can be used to treat colonic disorders, such as congenital anomalies (e.g., megacolon and imperforate anus), idiopathic disorders (e.g., diverticular disease and melanosis coli), vascular lesions (e.g., ischemic colistis, hemorrhoids, angiodysplasia), inflammatory diseases (e.g., colitis (e.g., idiopathic ulcerative colitis, pseudomembranous colitis), and lymphopathia venereum), Crohn's disease, and tumors (e.g., hyperplastic polyps, adenomatous polyps, bronchogenic cancer, colonic carcinoma, squamous cell carcinoma, adenoacanthomas, sarcomas, lymphomas, argentaffinomas, carcinoids, and melanocarcinomas).

General Classes of Disorders

For example, such molecules can be used to treat proliferative disorders, i.e., neoplasms or tumors (e.g., a carcinoma, a sarcoma, adenoma, or myeloid leukemia).

Disorders associated with abnormal TANGO-139, 125, 110, 175, or WDNM-2 activity or expression may include proliferative disorders (e.g., carcinoma, lymphoma, e.g., follicular lymphoma).

Disorders associated with abnormal TANGO-139, 125, 110, 175, or WDNM-2 activity or expression may include inflammatory disorders (e.g., bacterial infection, psoriasis, septicemia, cerebral malaria, inflammatory bowel disease (e.g., ulcerative colitis, Crohn's disease), arthritis (e.g., rheumatoid arthritis, osteoarthritis), and allergic inflammatory disorders (e.g., asthma, psoriasis)).

Disorders associated with abnormal TANGO-175, or WDNM-2 activity also include apoptotic disorders (e.g., rheumatoid arthritis, systemic lupus erythematosus, insulin-dependent diabetes mellitus).

Other TANGO-125, 110, 175, or WDNM-2 associated disorders may include differentiative and apoptotic disorders, and disorders related to angiogenesis (e.g., tumor formation and/or metastasis, cancer). Modulators of TANGO-125, 110, 175, or WDNM-2 expression and/or activity can be used to treat such disorders.

As integrin family members play a role in immune response, TANGO-175 or WDNM-2 nucleic acids, proteins, and modulators thereof can be used to treat immune related disorders, e.g., immunodeficiency disorders (e.g., HIV), viral disorders (e.g., infection by HSV), cell growth disorders, e.g., cancers (e.g., carcinoma, lymphoma, e.g., follicular lymphoma), autoimmune disorders (e.g., arthritis, graft rejection (e.g., allograft rejection), T cell autoimmune disorders (e.g., AIDS)), and inflammatory disorders (e.g., bacterial or viral infection, psoriasis, septicemia, cerebral malaria, inflammatory bowel disease (e.g., ulcerative colitis, Crohn's disease), arthritis (e.g., rheumatoid arthritis, osteoarthritis), allergic inflammatory disorders (e.g., asthma, psoriasis)).

As integrin family members play a role in cell growth, survival, proliferation, and migration, TANGO-175 or WDNM-2 nucleic acids, proteins, and modulators thereof can be used to treat apoptotic disorders (e.g., rheumatoid arthritis, systemic lupus erythematosus, insulin-dependent diabetes mellitus) proliferative disorders (e.g., cancers, e.g., B cell cancers stimulated by TNF), and disorders abnormal vascularization (e.g., cancer). In addition, TANGO-175 or WDNM-2 nucleic acids, proteins, and modulators thereof can also be used to promote vascularization (angiogenesis).

As integrins are cell adhesion molecules, TANGO-175 or WDNM-2 nucleic acids, proteins, and modulators thereof can be used to modulate disorders associated with adhesion and migration of cells, e.g., platelet aggregation disorders (e.g., Glanzmann's thromboasthemia, which is a bleeding disorders characterized by failure of platelet aggregation in response to cell stimuli), inflammatory disorders (e.g., leukocyte adhesion deficiency, which is a disorder associated with impaired migration of neutrophils to sites of extravascular inflammation), disorders associated with abnormal tissue migration during embryo development, and tumor metastasis.

Reproductive Disorders

TANGO-139, 125, 110, and 175 can be used to treat other reproductive disorders, including ovulation disorder, blockage of the fallopian tubes (e.g., due to pelvic inflammatory disease or endometriosis), disorders due to infections (e.g., toxic shock syndrome, chlamydia infection, Herpes infection, human papillomavirus infection), and ovarian disorders (e.g., ovarian cyst, ovarian fibroma, ovarian endometriosis, ovarian teratoma).

This invention is further illustrated by the following examples which should not be construed as limiting. The contents of all references, patents and published patent applications cited throughout this application are hereby incorporated by reference.

### EXAMPLES

### Example 1

### Isolation and Characterization of Human T139 cDNA

RNA was isolated from human fetal kidney tissue, and the polyA+ fraction was purified using Oligotex beads (Qiagen). Three micrograms of polyA+ RNA were used to synthesize a cDNA library using the Superscript cDNA Synthesis kit (Gibco BRL; Gaithersburg, Md.). Complementary DNA was directionally cloned into the expression plasmid pMET7 using the SalI and NotI sites in the polylinker to construct a plasmid library. Transformants were picked and grown for single-pass sequencing. One cDNA clone (jthKa115e09) was identified that encoded a protein with homology to testis-specific protein-1 (TPX-1), an acrosomal sperm protein that is a member of the SCP-like family of cysteine-rich secreted proteins. JthKa115e09 contains an open reading frame of 446 amino acids, which is referred to as “Tango 139”.

### Example 2

### Distribution of T139 mRNA in Human Tissues

The expression of T139 was analyzed using Northern blot hybridization. Oligonucleotide primers (5′CCATGCTGCATCCAGAG 3′ (SEQ ID NO:7); 5′ CACAGACAAAGGCTTCTATC 3′ (SEQ ID NO:8)) were used to amplify a 543 bp fragment from the coding region of jthKa114e09, and the DNA was radioactively labeled with ³²P-dCTP using a Prime-It kit (Stratagene, La Jolla, Calif.) according to the supplier's instructions. Filters containing human mRNA (MTNI and MTNII from Clontech, Palo Alto, Calif.) were probed in ExpressHyb hybridization solution (Clontech) and washed at high stringency according to manufacturer's recommendations.

Tango 139 is expressed at high levels as a transcript of about 2.0 kb in the kidney, with lower levels in the testis. In addition, there are additional transcripts in both kidney and testis at about 2.4 and 3.5 kb. No other tissues examined (heart, brain, placenta, lung, liver, skeletal muscle, pancreas, spleen, thymus, ovaries, small intestine, colon and peripheral blood leukocytes) showed any expression.

### Example 3

### Characterization of T139 Proteins

In this example, the predicted amino acid sequence of human T139 protein was compared to amino acid sequences of known proteins and various motifs were identified. In addition, the molecular weight of the human T139 proteins was predicted.

The human T139 cDNA isolated as described above (FIG. 1; SEQ ID NO:1) encodes a 446 amino acid protein (FIG. 1; SEQ ID NO:2). The signal peptide prediction program SIGNALP (Nielsen et al. (1997) Protein Engineering 10: 1-6) predicted that T139 includes a 26 amino acid signal peptide (amino acid 1 to about amino acid 26 of SEQ ID NO:2) preceding the 420 amino acid mature protein (about amino acid 27 to amino acid 446; SEQ ID NO:4). A hydropathy plot of T139 is presented in FIG. 3. This plot shows the location of cysteines (“cys”; short vertical lines just below plot) and the PFAM identifiers (PF00188, PF00008, and PF00059; bars just above plot). For general information regarding PFAM identifiers refer to Sonnhammer et al. (1997) Protein 28:405-420 and http://www.psc.edu/general/software/packages/pfam/pfam.html.

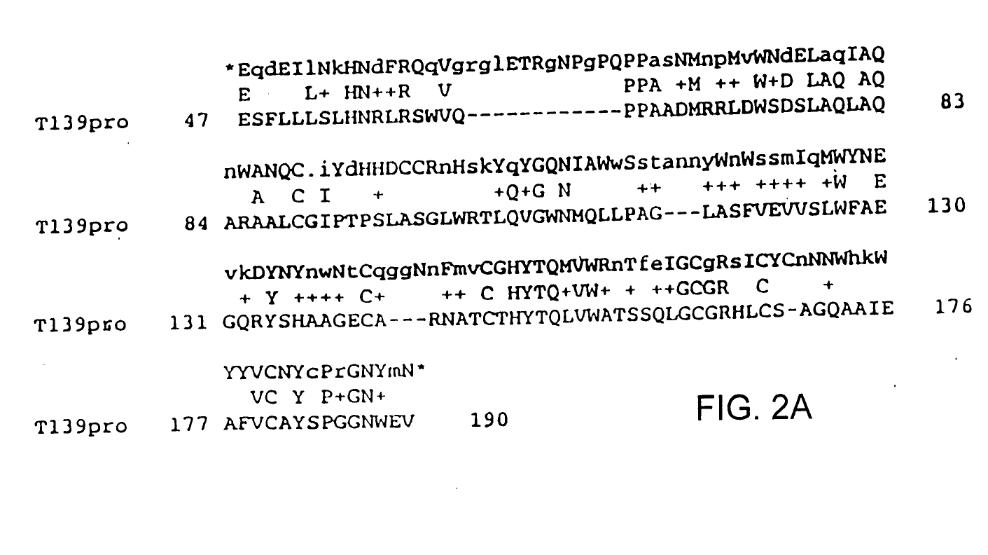

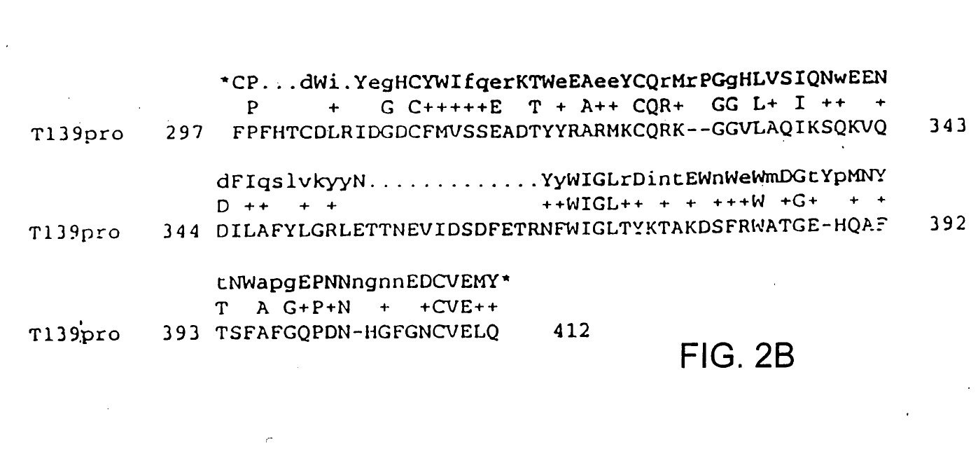

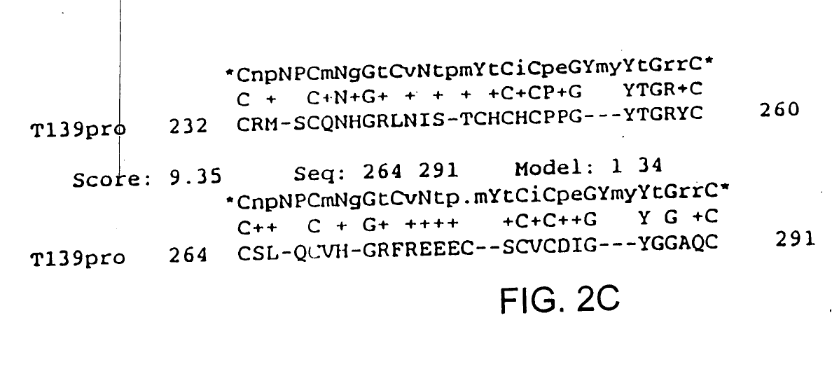

As shown in FIG. 2A, T139 has a region of homology (amino acids 47 to 190, of SEQ ID NO:2) to a SCP-like domain consensus sequence (PF00188, of SEQ ID NO:2). FIG. 2B shows the region of homology (amino acids 297 to 412, SEQ ID NO:2) to the C-type lectin domain consensus sequence (PF00059). Although significant homology was observed, the four cysteines in this region of T139 do not match the four conserved cysteines in the consensus sequence; the alignment only recognized three of the four cysteines in this region of T139 as identical to the consensus cysteines. FIG. 2C shows the regions of homology (amino acids 232 to 260 of SEQ ID NO:2; (EGF1) and 264 to 291 of SEQ ID NO:2; (EGF2)) to a EGF-like domain consensus sequence (PF00008). Although both the EGF1 and EGF2 domains contain six cysteines as does the consensus, the alignment only recognizes five cysteines as matching the consensus. Mature T139 has a predicted MW of 49 kDa (47 kDa with the signal peptide removed), not including post-translational modifications. A signal peptide is predicted to exist from amino acids 1 to 26, using the prediction program SIGNALP (Nielsen et al. (1997) Protein Engineering 10:1-6).

### Example 4

### Preparation of T139 Proteins

Recombinant T129 can be produced in a variety of expression systems. For example, the mature T129 peptide can be expressed as a recombinant glutathione-S-transferase (GST) fusion protein in E. coli and the fusion protein can be isolated and characterized. Specifically, as described above, T129 can be fused to GST and this fusion protein can be expressed in E. coli strain PEB 199. Expression of the GST-T129 fusion protein in PEB 199 can be induced with IPTG. The recombinant fusion protein can be purified from crude bacterial lysates of the induced PEB 199 strain by affinity chromatography on glutathione beads.

### Example 5

### Isolation and Characterization of Human T125 cDNAs

Human aortic endothelial cells (obtained from Clonetics Corporation; San Diego, Calif.) were expanded in culture with Endothelial Cell Growth Media (EGM; Clonetics) according to the recommendations of the supplier. When the cells reached ˜80-90% confluence, they were stimulated with TNF (10 ng/ml) and cycloheximide (CFI; 40 micrograms/ml) for 4 hours. Total RNA was isolated using the RNeasy Midi Kit (Qiagen; Chatsworth, Calif.), and the poly A+ fraction was further purified using Oligotex beads (Qiagen).

Three micrograms of poly A+ RNA were used to synthesize a cDNA library using the Superscript cDNA Synthesis kit (Gibco BRL; Gaithersburg, Md.). Complementary DNA was directionally cloned into the expression plasmid pMET7 using the SalI and NotI sites in the polylinker to construct a plasmid library. Transformants were picked and grown up for single-pass sequencing. A partial cDNA clone (jthdc042c10) was identified that encoded a protein with homology to a Genbank entry (gi-1841553) which appeared to encode a secreted protein with two EGF domains (note: This Genbank entry seems to be a condensation of genomic sequence relying on EST sequence to define the coding region, and there may be some errors or alternative splicing within the entry.) Jthdc042c10 was completely sequenced, and lacked an appropriate start codon. Therefore additional homologous clones in the library were identified by database searches and sequenced. One clone (jthdc054a01) contained a 273 amino acid open reading frame that was ˜37% identical with gi-1841553, and contained a predicted signal sequence (amino acids 1-22). Two regions of Tango 125 showed similarity to EGF domains (amino acids 107-134 and amino acids 141-176 of SEQ ID NO:10), and there was complete conservation of all cysteines between Tango 125 and gi-1841553.

### Example 6

### Distribution of T125 mRNA in Human Tissues

The expression of T125 was analyzed using Northern blot hybridization. Primers (5′ GCTCACGGGGACCCTGTC 3′ (SEQ ID NO:27) and 5′CAGTGCCTGCGAGGCCAG 3′ (SEQ ID NO:28)) were used to amplify a 585 bp fragment from the 5′ end of the T125 coding region. The DNA was radioactively labeled with ³²P-dCTP using the Prime-It kit (Stratagene, La Jolla, Calif.) according to the instructions of the supplier. Filters containing human mRNA (MTNI and MTNII from Clontech, Palo Alto, Calif.) were probed in ExpressHyb hybridization solution (Clontech) and washed at high stringency according to manufacturer's recommendations.

T125 is expressed as series of transcripts between 1.3 and 3 kb. These transcripts are found at variable levels in all tissues examined (spleen, thymus, prostate, testes, ovary, small intestine, colon, heart, brain, placenta, lung, liver, skeletal muscle, kidney and pancreas) with the exception of peripheral blood leukocytes in which expression was not detected. The highest levels of T125 expression were observed in the placenta as a 3 kb transcript, with the next highest levels found in spleen and testis as ˜2 and 1.5 kb transcripts respectively.

The various size transcripts seen on the Northern blots could be consistent with alternative splicing of the T125 gene. Although there were no changes in the coding region between the clones that were sequenced, the clones appeared to be partially spliced transcripts. It is unknown at this point if the alternative splicing is important for the regulation of expression, or whether additional clones containing variations in the coding sequence may also be expressed.

Human in situ expression analysis revealed that T125 is expressed in lung (ubiquitous with multifocal areas of higher expression), thymus (ubiquitous with multifocal areas of higher expression), heart, kidney, liver, non-follicular regions of the spleen. Expression was also observed, at a lower level, in brain and placenta. In situ expression analysis of human embryonic tissues revealed that T125 is expressed in most tissues with the highest expression in heart, lung, kidney, and early fetal liver (E13.5 through E15.5).

### Example 7

### Characterization of T125 Proteins

In this example, the predicted amino acid sequence of human T125 protein was compared to amino acid sequences of known proteins and various motifs were identified. In addition, the molecular weight of the human T125 proteins was predicted.

The human T125 cDNA isolated as described above (FIG. 4; SEQ ID NO:9) encodes a 273 amino acid protein (FIG. 4; SEQ ID NO:10). The signal peptide prediction program SIGNALP (Nielsen et al. (1997) Protein Engineering 10: 1-6) predicted that T125 includes a 22 amino acid signal peptide (amino acid 1 to about amino acid 22 of SEQ ID NO:2) preceding the 252 amino acid mature protein (about amino acid 23 to amino acid 274; SEQ ID NO:12).

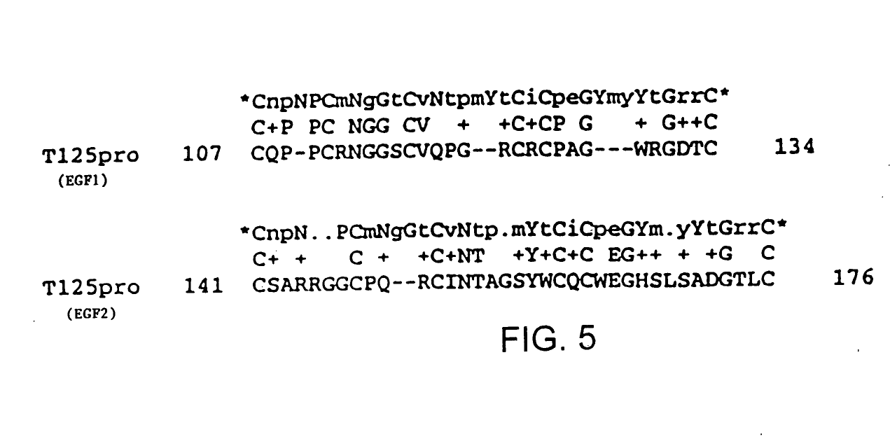

As shown in FIG. 5, T125 has two regions of homology (amino acids 107 to 134 of SEQ ID NO:10; and amino acids 141 to 176 of SEQ ID NO:10) to the EGF-like domain consensus sequence. Both regions contain the six conserved cysteines and two conserved glycines between the fifth and sixth cysteine in the consensus sequence. The mature T125 protein is predicted to have a MW of 30 kDa (27 kDa without the signal peptide).

### Example 8

### Alternatively Spliced Forms of Human T125

Additional analysis revealed that the human T125 cDNA shown in FIG. 4 represents one of four alternatively spliced forms of human T125. The three additional forms, T125a, T125b, and T125c are depicted in FIG. 8, FIG. 9, and FIG. 10 respectively. FIG. 8 depicts the cDNA sequence (SEQ ID NO:16) and predicted amino acid sequence (SEQ ID NO:17) of human T125a. The open reading frame of SEQ ID NO:16 extends from nucleotide 194 to nucleotide 442 of SEQ ID NO:16 (SEQ ID NO:18). FIG. 9 depicts the cDNA sequence (SEQ ID NO:19) and predicted amino acid sequence (SEQ ID NO:20) of human T125b. The open reading frame of SEQ ID NO:19 extends from nucleotide 194 to nucleotide 934 of SEQ ID NO:19 (SEQ ID NO:21). FIG. 10 depicts the cDNA sequence (SEQ ID NO:22) and predicted amino acid sequence (SEQ ID NO:23) of T125c. The open reading frame of SEQ ID NO:22 extends from nucleotide 194 to nucleotide 823 of SEQ ID NO:22 (SEQ ID NO:24).

The four forms arise from the use of three exons. All four forms include exon 1. The form of human T125 (called T125) depicted in FIG. 4 includes exon 2 and exon 3 in addition to exon 1. T125a includes exon 2 in addition to exon 1. T125b includes exon 1 only. T125c includes exon 3 in addition to exon 1. The coding sequence of both T125b and T125c begins at an ATG that is upstream of the ATG that is the beginning of the coding sequence for T125 and T125b. T125 may be subject to two types of post-transcriptional regulation: choice of initiation site and choice of splicing.

### Example 9

### Identification of Murine T125 and Distribution of T125 in Murine Tissue

A full-length murine T125 cDNA clone was isolated. This 846 nucleotide cDNA is depicted in FIG. 7 (SEQ ID NO:13). The open reading frame of this molecule extends from nucleotide 13 to nucleotide 837 of SEQ ID NO:13 (SEQ ID NO:14) and encodes a 275 amino acid protein (SEQ ID NO:15).

Northern blot analysis revealed that murine T125 is expressed at a moderate level in heart, lung, and liver and at a lower level in brain and kidney.

In situ expression analysis revealed that murine T125 is expressed in lung (ubiquitous with multifocal areas of higher expression), thymus (ubiquitous with multifocal areas of higher expression), liver (ubiquitous with probable expression in hepatocytes), kidney (ubiquitous), spleen (non-follicular), brain (low, but ubiquitous), placenta (ubiquitous, inner mass). In situ expression analysis of murine embryonic tissue revealed ubiquitous expression at E13.5 through E15.5, with higher expression in lung, heart, liver, and kidney. At E16.5 through E18.5 and at P1.5, the ubiquitous expression of T125 decreases with higher signal persisting in lung, heart, and kidney.

Overexpression of murine T125 in mice using a retroviral expression system revealed the T125 overexpression may reduce triglyceride levels by nearly 50%.

### Example 10

### Preparation of T125 Proteins

Recombinant T125 can be produced in a variety of expression systems. For example, the mature T125 polypeptide can be expressed as a recombinant glutathione-S-transferase (GST) fusion protein in E. coli, and the fusion protein can be isolated and characterized. Specifically, as described above, T125 can be fused to GST, and this fusion protein can be expressed in E. coli strain PEB199. Expression of the GST-T125 fusion protein in PEB 199 can be induced with IPTG. The recombinant fusion protein can be purified from crude bacterial lysates of the induced PEB199 strain by affinity chromatography using glutathione beads.

### Example 11

### Creation of Flag-tagged T125

A flag epitope-tagged version of T125 is constructed by PCR amplifying a T125 gene using a 3′ primer that includes a nucleotide sequence encoding the DYKDDDDK flag epitope (SEQ ID NO:68) followed by a termination codon. The amplified clone is inserted into a pMET vector and the resulting construct is used to transiently transfected into HEK 293T cells in 150 mM plates using Lipofectamine (GIBCO/BRL, Gaithersburg Md.) according to the manufacturer's protocol. The cells are used to express flag-tagged T! @%.

### Example 12

### Retroviral Delivery of T125

Full length human or murine T125 is expressed in vivo mediated by retroviral infection. A sequence encoding a selected T125 is cloned into the retroviral vector MSCVneo (Hawley et al. (1994) Gene Therapy 1:136-138), and sequence verified. Bone marrow from 5-fluorouracil treated mice infected with the retrovirus is then transplanted into irradiated mouse recipients.

### Example 13

### T125 alkaline phosphatase N-terminal fusion protein

A vector expression a T125-alkaline phosphatase fusion protein is prepared by ligating a sequence encoding a selected T125 into AP-Tag3 vector (Tartaglia et al. (1995) Cell 83:1263-1271). The full-length open-reading frame of T125 is PCR amplified using a 5′ primer incorporating a BglII restriction site prior to the nucleotides encoding the first amino acids of T125 and a 3′ primer including a XhoI restriction site immediately following the termination codon of T125. Thus the open reading frame of the complete construct includes the complete sequence of human placental alkaline phosphatase, including the signal peptide, followed by T125 sequence.

The resulting vector is transiently transfected into HEK 293T cells in 150 mM plates using Lipofectamine (GIBCO/BRL) according to the manufacturer's protocol. Seventy-two hours post-transfection, the serum-free conditioned media (OptiMEM, GIBCO/BRL) is harvested, spun and filtered. Alkaline phosphatase activity in conditioned media is quantitated using an enzymatic assay kit (Phospha-Light, Tropix Inc.) according to the manufacturer's instructions. Conditioned medium samples are analyzed by SDS-PAGE followed by Western blot using anti-human alkaline phosphatase antibodies diluted 1:250 (Genzyme Corp., Cambridge Mass.) and detected by chemiluminescence.

### Example 14

### Isolation and Characterization of Human T110 cDNAs

A cDNA library was prepared from polyA mRNA isolated from ratPC12 cells (PC6-3 subline) that had been cultured in the absence of neurotrophic factors (NGF) for 12 hours. Random 5′ sequencing yielded a single clone with homology to the D. melanogaster fj gene. This partial rat clone was used to screen mouse and human fetal brain cDNA libraries. These screens have yielded clones containing mouse T110 and human Ti 10.

Complete sequencing of the human T110 clone revealed an approximately 2.4 kb cDNA insert with a ¹³¹I base pair open reading frame predicted to encode a novel secreted protein, i.e., human T110. Complete sequencing of the mouse T110 clone revealed an approximately 2.1 kb cDNA insert with a 1350 base pair open reading frame predicted to encode a novel secreted protein, i.e., mouse T110. The mouse and human protein sequences are about 85% identical. The major region of divergence is towards the N-terminus.

FIG. 16 depicts the cDNA sequence (SEQ ID NO:29) and predicted amino acid sequence (SEQ ID NO:32) of a potential alternative human T110 translation product. The open reading frame extends from nucleotide 2 to 1441 of SEQ ID NO:29).

FIG. 18 depicts the cDNA sequence (SEQ ID NO:33) and predicted amino acid sequence (SEQ ID NO:36) of a potential alternative murine T110 translation product. The open reading frame extends from nucleotide 1 to 1452 of SEQ ID NO:33.

### Example 15

### Distribution of T110 mRNA in Human Tissues

The expression of T110 was analyzed using Northern blot hybridization. In rat, the Northern blot analysis of adult tissues showed highest expression in brain and kidney. Expression was also observed in heart and lung. No mRNA was detected in spleen, liver, skeletal muscle or testis.

To examine the tissue distribution of human T110, the rat partial cDNA sequence was used as a probe for the Northern blot analysis. The cDNA was radioactively labeled with ³²P-dCTP using the Prime-It kit (Stratagene; La Jolla, Calif.) according to the instructions of the supplier. Filters containing human mRNA (MTNI and MTNII: Clontech; Palo Alto, Calif.) were probed in ExpressHyb hybridization solution (Clontech, Palo Alto, Calif.) and washed at high stringency according to manufacturer's recommendations.

These studies revealed that human T110 was expressed as an approximately 2.4 kilobase transcript at highest level in brain, heart, placenta, and pancreas. Lower levels of transcript were seen in liver, skeletal muscle, and kidney. Transcript was not detected in lung. Embryonic expression was seen in week 8-9 fetus and week 20 liver and spleen mixed tissue.

In situ expression assays on mouse embryos revealed that T110 is expressed in the nervous system. In adult mice, in situ expression assays revealed that T110 is expressed in discrete regions of the brain, including the cerebellum and olfactory bulb, and in the non-islet cells of the pancreas.

### Example 16

### Characterization of T110 Proteins

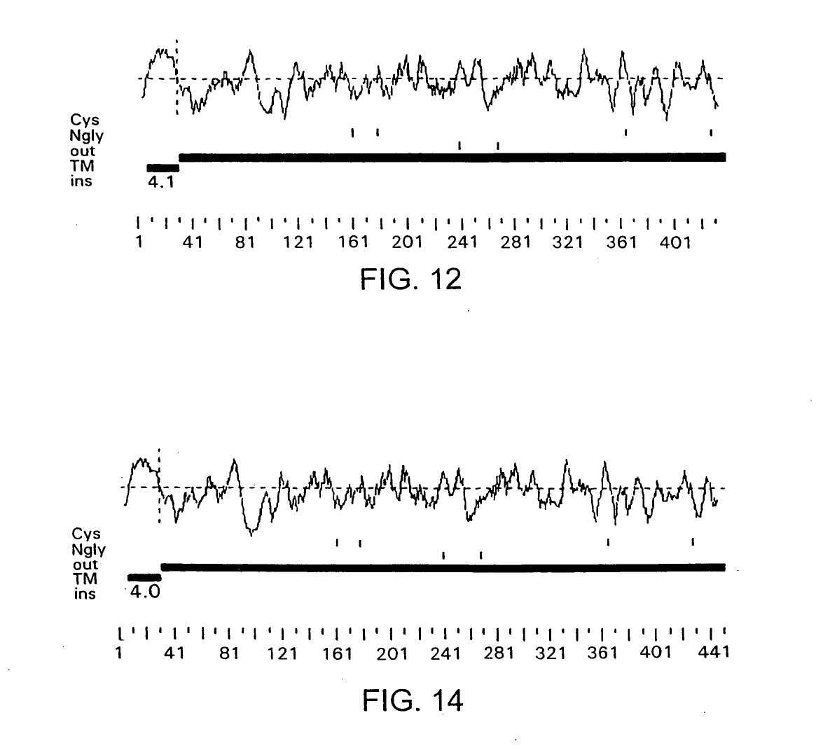

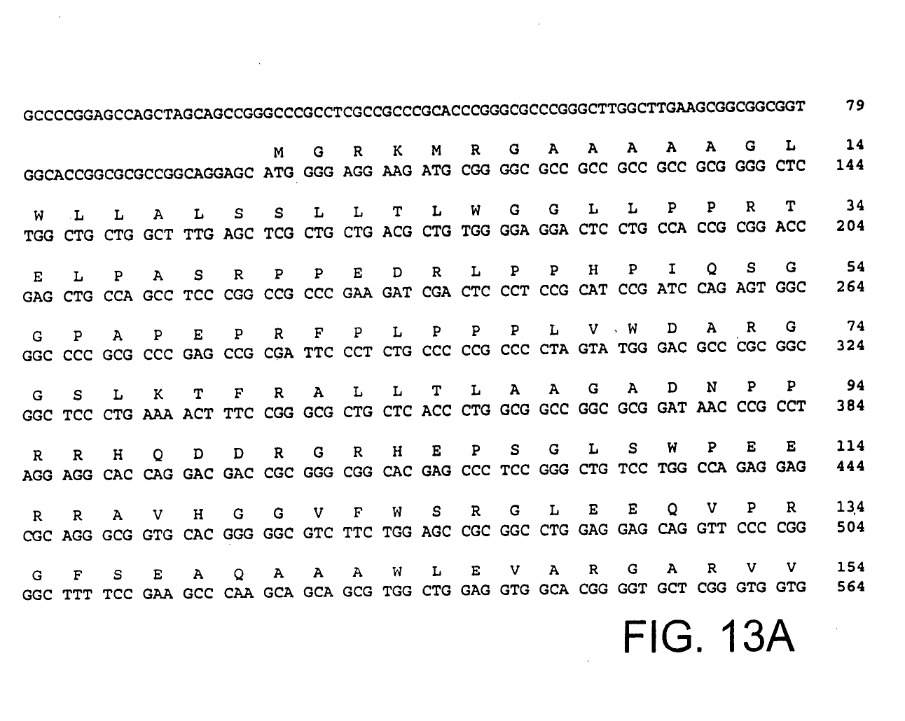

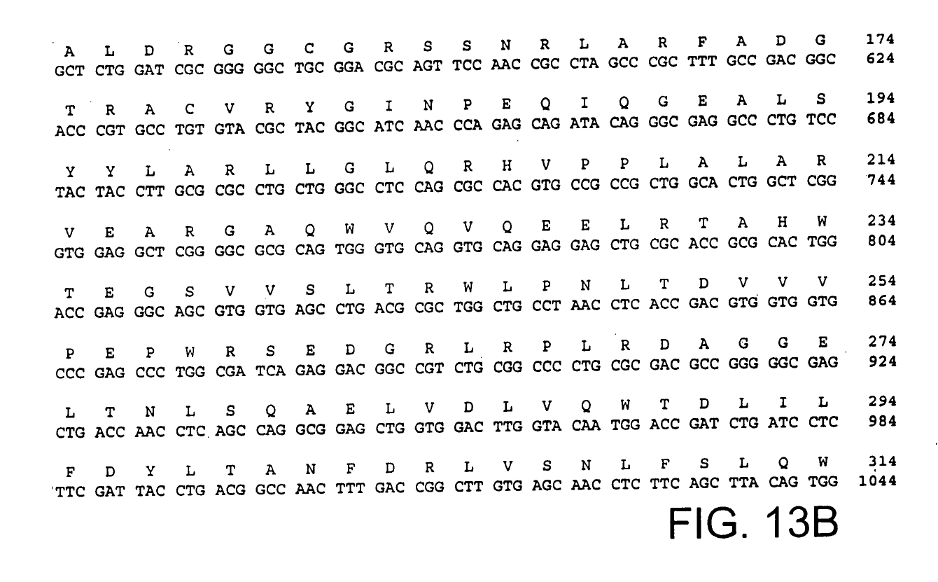

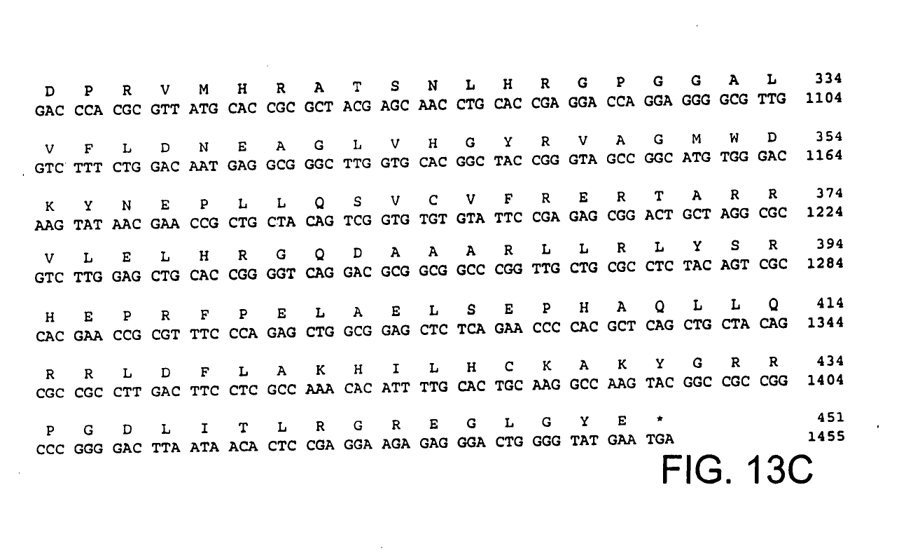

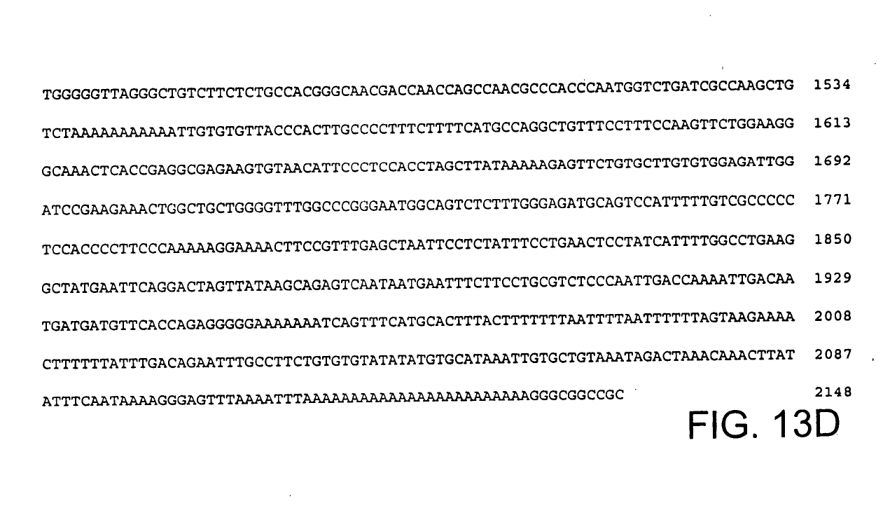

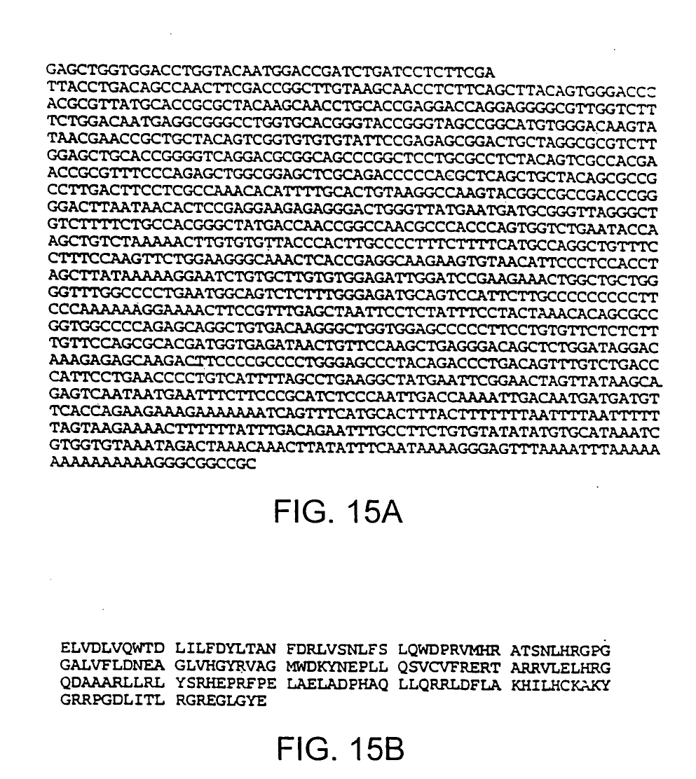

The human T110 cDNA (FIG. 11; SEQ ID NO:29) isolated as described above encodes a 437 amino acid protein (FIG. 11; SEQ ID NO:30). A hydropathy plot of T110 is presented in FIG. 12. This plot shows the presence of a signal sequence (amino acids 1-28) and a hydrophobic region that may indicate a transmembrane domain (amino acid 7-30) that acts as an internal signal sequence.

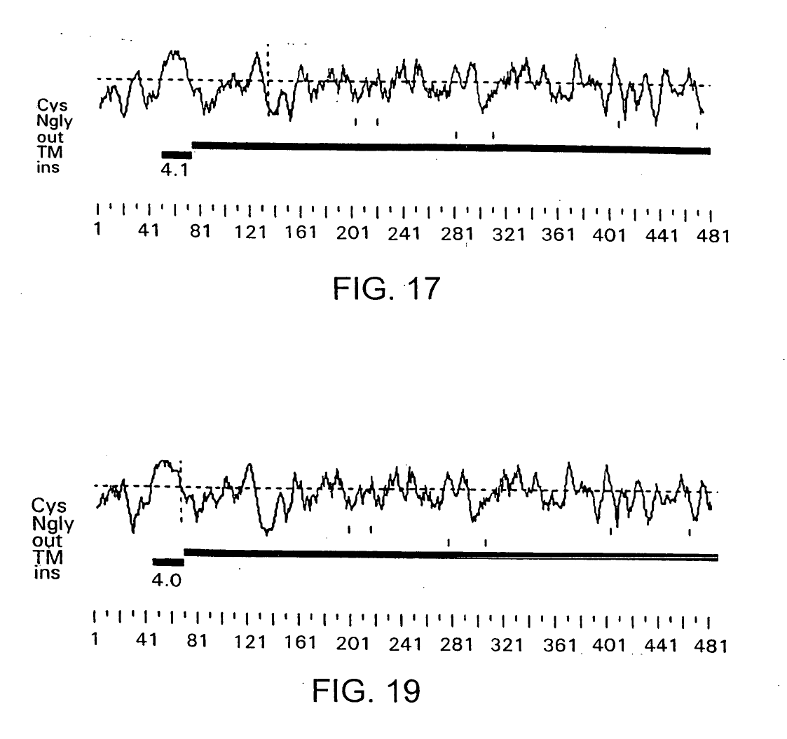

FIG. 17 is a plot showing predicted structural features of a potential alternative human T110 protein (SEQ ID NO:32). This figure shows predicted alpha helix regions (Garnier-Robson and Chou-Fasman), predicted beta sheet regions (Garnier-Robson and Chou-Fasman), predicted turn regions (Gamier-Robson and Chou-Fasman), predicted coil regions (Garnier-Robson), predicted hydrophilicity, predicted alpha amphipathic regions (Eisenberg) predicted beta amphipathic regions (Eisenberg), predicted flexible regions (Karplus-Schultz), predicted antigenic index (Jameson-Wolf), and surface probability (Emini).

A sequence alignment of human T110 protein and D. melanogaster fj protein, as shown in FIG. 16, reveals that both proteins are of similar size, contain a single predicted hydrophobic region as the transmembrane and internal signal sequence, and include a large extracellular domain with two pairs of conserved cysteine residues. In this alignment, which includes gaps, the proteins are 20.7% identical and 35.9% similar.

Mature human T110 has a predicted MW of 48 kDa, not including post-translational modifications.

A secretion assay revealed that T110 is a secreted protein. It may be secreted using a signal peptide (amino acids 1-28) or a transmembrane region (amino acids 7-30) that acts as an internal signal sequence.

### Example 17

### Preparation of T110 Proteins

Recombinant T110 can be produced in a variety of expression systems. For example, the mature T110 peptide can be expressed as a recombinant glutathione-S-transferase (GST) fusion protein in E. coli and the fusion protein can be isolated and characterized. Specifically, as described above, T 110 can be fused to GST and this fusion protein can be expressed in E. coli strain PEB 199. Expression of the GST-T110 fusion protein in PEB199 can be induced with IPTG. The recombinant fusion protein can be purified from crude bacterial lysates of the induced PEB 199 strain by affinity chromatography on glutathione beads.

### Example 18

### Identification and Characterization of Murine and Human TANGO-175 cDNAs

A partial cDNA encoding murine TANGO-175 was identified by subtractive cDNA hybridization using stimulated and unstimulated bone marrow cells. The bone marrow cells were obtained from the femurs of adult C57BL/6 female mice following the procedure of StemCell Technologies, Inc. (StemCell Technologies, Inc., Vancouver, Canada) with minor changes. Briefly, bone marrow was flushed from the femurs using phosphate buffered saline (PBS), pH 7.4, supplemented with 5% heat-inactivated fetal calf serum (PBS/5% HIFCS). After creating a single cell suspension by repetitive pipetting of the bone marrow, the cells were washed once in PBS/5% HIFCS, and the red blood cells were lysed by incubation with 3M ammonium chloride for 3 minutes on ice. Following termination of lysis by addition of PBS/5% HIFCS, the bone marrow cells were washed once more with PBS/5% HIFCS and plated at 8×10⁷ cells/20 ml/75 cm² flask in murine myeloid long-term culture medium (MyeloCult™ M5300, StemCell Technologies, Inc., Vancouver, Canada). The cultures were incubated at 33° C. in a 5% CO₂ humidified chamber for three weeks. Half the medium was replaced weekly with fresh medium. Following 3 weeks of incubation, the bone marrow cultures were stimulated for 2 hours at 33° C. with 50 ng/ml phorbol 12-myristate 13-acetate (TPA; Sigma, Inc.) and 1 μM ionomycin (Sigma, Inc.).

Total RNA was then isolated from stimulated bone marrow cells, and from unstimulated sister cultures, using Qiagen RNeasy Maxi Kit (Qiagen, Inc.). The polyA+ RNA was isolated from each total RNA pool using the Oligotex mRNA Kit (Qiagen, Inc.) and then treated with RNase-free DNase (Boehringer Mannheim).

The DNase-treated, polyA+ RNA was subjected to “PCR select” using the PCR-Select cDNA Subtraction Kit (Clontech, Inc.). The cDNA of unstimulated bone marrow cells was obtained and subtracted from that of stimulated bone marrow cells. The PCR-amplified, differentially expressed cDNA was subcloned using TA Cloning Kit (Invitrogen, Inc.), transformed into ElectroMAX DH10B cells (Gibco BRL) and plated onto LB/amp plates. The DNA from individual transformant colonies was isolated and sequenced using an automated sequencer. The clone sequences were analyzed by comparison to available protein databases using the BLAST algorithm.

One clone, etmM031 (encoding the amino acid sequqnce shown in FIG. 26; SEQ ID NO:60), was found encode a protein (later named TANGO-175) having significant homology to murine the WDNM-1 protein (Dear and Kefford (1991) Biochem. Biophys. Res. Comm. 176:247-54; FIG. 26, SEQ ID NO:58).

The nucleotide sequence of clone etmM013 was used to search the IMAGE EST database. This search led to the identification of EST W11247. A clone corresponding to this EST was fully sequenced (FIG. 22; SEQ ID NO:43) and found to encode full-length murine TANGO-175. This clone was used to search the human IMAGE EST database in an effort to identify an EST having homology to the murine TANGO-175 cDNA described above. This search led to the identification of EST W52431. A clone corresponding to EST W52431 was fully sequenced (FIG. 30; SEQ ID NO:62). This clone does not appear to encode a human homologue of murine TANGO-175. However, analysis of the three potential reading frames of the clone suggested that a change in the reading frame just after nucleotide 49 would result in the encoding of a protein with considerable homology to murine TANGO-175 protein. Based on this analysis four human TANGO-175 cDNAs, all of which encode the same protein, were devised.

The four cDNAs encoding human TANGO-175 (FIGS. 23A-D; SEQ ID NOs:46-49; SEQ ID NOs:50-53, open reading frame only) all encode the same protein (FIGS. 23A-D; SEQ ID NO:54) and differ only in the codon encoding amino acid 10. The cDNAs are 501 nucleotides long, including untranslated regions, and have a 183 nucleotide open reading frame (nucleotides 23-204 of SEQ ID NOS:46-49, SEQ ID NOS:50-53) which encodes a 61 amino acid protein (SEQ ID NO:54). Based on the sequence of the clone corresponding to EST 52431, FIG. 23A is thought most likely to represent a naturally occurring cDNA encoding human Tango-175. Human TANGO-175 protein is predicted to be a 4 kDa protein (excluding post-translational modifications).

### Example 19

### Distribution of TANGO-175 mRNA in Human and Murine Tissues

The expression patterns of murine and human TANGO-175 were analyzed using Northern blot hybridization.

An approximately 0.5 kb murine TANGO-175 mRNA transcript was identified in liver, spleen, heart, kidney, and skeletal muscle. The expression in liver was far higher than in spleen, heart, kidney, or skeletal muscle

An approximately 0.5 kb human TANGO-175 mRNA transcript was identified in lymph node, spleen, thymus, uterus, and lung.

Endogenous murine TANGO-175 gene expression was determined using the Perkin-Elmer/ABI 7700 Sequence Detection System which employs TaqMan technology. Briefly, TaqMan technology relies on standard RT-PCR with the addition of a third gene-specific oligonucleotide (referred to as a probe) which has a fluorescent dye coupled to its 5′ end (typically 6-FAM) and a quenching dye at the 3′ end (typically TAMRA). When the fluorescently tagged oligonucleotide is intact, the fluorescent signal from the 5′ dye is quenched. As PCR proceeds, the 5′ to 3′ nucleolytic activity of taq polymerase digest the labeled primer, producing a free nucleotide labeled with 6-FAM, which is now detected as a fluorescent signal. The PCR cycle where fluorescence is first released and detected is directly proportional to the starting amount of the gene of interest in the test sample, thus providing a way of quantitating the initial template concentration. Samples can be internally controlled by the addition of a second set of primers/probe specific for a housekeeping gene such as GAPDH which has been labeled with a different fluor on the 5′ end (typically JOE).

To determine the level of TANGO-175 in various murine tissues a primer/probe set was designed using Primer Express software and primary cDNA sequence information. Total RNA was prepared from a series of murine tissues using an RNeasy kit from Qiagen. First strand cDNA was prepared from one ug total RNA using an oligo dT primer and Superscript II reverse transcriptase (Gibco/BRL). cDNA obtained from approximately 50 ng total RNA was used per TaqMan reaction. Normal tissues tested include mouse brain, heart, liver, lung, spleen, testis, kidney and megakaryocytes. Expression was greatest in liver (approximately 10-fold greater than that signal seen for GAPDH) followed by spleen, megakaryocytes and lung. TANGO-175 was expressed weakly in testis, heart and kidney and absent in total brain.

In situ hybridization analysis in mice revealed that TANGO-175 is expressed hepatocytes. Within the liver expression was not detected in vascular endothelium and associated muscle cells, mesenchymal cells of the capsule, and areas of extramedullary hematopoesis. This same analysis revealed that TANGO-175 appears to be ubiquitously expressed in adult thymus. In situ expression analysis in mice revealed that TANGO-175 is expressed in fetal liver beginning at day E14.5. Expression in this tissue increases to a maximum at day E16.5 and stays at that level at least through post-natal day 1.5. In this analysis, expression was not detected in pancreas, placenta, eye, heart, thymus, spleen, kidney, lung, brain, colon, small intestine, skeletal muscle, and smooth muscle.

### Example 20

### Identification and Characterization of Murine WDNM-2

Using a composite nucleotide sequence based on the nucleotide sequences of human TANGO-175, murine TANGO-175, and rat WDNM-1, the IMAGE EST database was searched in an effort to identify clones which might encode unknown proteins having homology to human and murine TANGO-175. This search led to the identification of clone mine17967 (FIG. 24; SEQ ID NO:55, SEQ ID NO:57 open reading frame only). This clone is predicted to encode a 76 amino acid protein (SEQ ID NO:56) later named WDNM-2.

### Example 21

### Characterization of TANGO-175 and Murine WDNM-2

In this example, the predicted amino acid sequence of the TANGO-175 proteins and murine WDNM-2 are compared to amino acid sequences of known proteins and various motifs are identified.

The murine TANGO-175 cDNA (SEQ ID NO:43) has a 189 nucleotide open reading frame (nucleotides 18-206 of SEQ ID NO:43; SEQ ID NO:45) which encodes a 63 amino acid protein (SEQ ID NO:44). This protein includes a predicted signal sequence of about 24 amino acids (from amino acid 1 to about amino acid 24 of SEQ ID NO:44) and a predicted mature protein of about 39 amino acids (from about amino acid 25 to amino acid 63 of SEQ ID NO:44; SEQ ID NO:63). Murine TANGO-175 protein possesses six cysteine residues which form interdomain bonds which stabilize the protein and are likely to be essential for biological activity. The six cysteine residues, C1-C6, occur at amino acid 35, 39, 45, 51, 56 and 60 of SEQ ID NO:44, respectively. Murine TANGO-175 also includes an RGD motif, which likely mediates cell attachment to the TANGO-175 protein.

Murine TANGO-175 protein has some sequence similarity to the amino acid sequence of murine WDNM-1 (mWDNM-1; SEQ ID NO:58), rat WDNM-1 (rWDNM; SEQ ID NO:59), and murine anti-leukoproteinase (mALP; SEQ ID NO:61) (FIG. 26).

A search for regions with homology to an identified Hidden Markov Motif identified amino acids 23-63 of murine TANGO-175 as having homology to PF00095, corresponding Whey Acidic Protein ‘four-disulfide core’. This search also identified amino acids 34-60 of murine TANGO-175 as having homology to PF000396, corresponding to granulin. For general information regarding Hidden Markov Motifs, refer to Sonnhammer et al. (1997 Protein 28:405-420) and http://www.psc.edu/general/software/packages/pfam/pfam.html.

The nucleotide sequences encoding human TANGO-175 (FIGS. 23A, 23B, 23C, and 23D; SEQ ID NO:4649) encode a 61 amino acid protein (FIGS. 23A-D; SEQ ID NO:54). The signal peptide prediction program SIGNALP Optimized Tool (Nielsen et al. (1997) Protein Engineering 10:1-6) predicted that TANGO-175 includes a 24 amino acid signal peptide (amino acid 1 to about amino acid 24 of SEQ ID NO:54) preceding the mature protein (about amino acid 25 to amino acid 61; SEQ ID NO:54; SEQ ID NO:64).

Human TANGO-175 contains a three-disulfide core pattern of cysteines found in murine TANGO-175. Thus, human TANGO-175 protein possesses six cysteine residues, cysteines C1-C6, which occur at amino acids 33, 37, 43, 49, 54 and 58 of SEQ ID NO:54, respectively. These cysteine residues form interdomain disulfide bonds which stabilize the human TANGO-175 protein. Cysteines C1-C5, C2-C4 and C3-C6 pair to form disulfide bonds. Like murine TANGO-175, human TANGO-175 protein has some sequence similarity to murine WDNM-1 (mWDNM-1; SEQ ID NO:58), rat WDNM-1 (rWDNM; SEQ ID NO:59), and murine anti-leukoproteinase (mALP; SEQ ID NO:61) (FIG. 26).

A search for regions with homology to an identified Hidden Markov Motif identified amino acids 22-61 of human TANGO-175 as having homology to PF00095, corresponding Whey Acidic Protein ‘four-disulfide core’. This search also identified amino acids 32-58 of human TANGO-175 as having homology to PF000396, corresponding to granulin.

FIG. 26 is an alignment of the amino acid sequence of murine WDNM-2 (SEQ ID NO:56) with murine WDNM-1 (mWDNM-1; SEQ ID NO:58), rat WDNM-1 (rWDNM; SEQ ID NO:59), etmM031 (SEQ ID NO:60), murine TANGO-175 (mT.175orf; SEQ ID NO:44), human TANGO-175 (hT.175prot; SEQ ID NO:54), and murine anti-leukoproteinase (mALP; SEQ ID NO:61). Based on this alignment, Murine TANGO-175 has 15 residues identical to rat WDNM-1; 16 residues identical to murine WDNM-1; and 19 residues identical to murine anti-leukoproteinase. Similarly as shown in FIG. 26, human Tango-175 has 19 residues identical to rat WDNM-1; 20 residues identical to mouse WDNM-1; 12 residues identical to murine anti-leukoproteinase. FIG. 26 also shows that WDNM-2 has 37 residues identical to rat WDNM-1; 51 residues identical to murine WDNM-1; and 25 residues identical to murine anti-leukoproteinase.

### Example 22

### Assay Confirming that TANGO-175 is Secreted

Secretion assays reveal that human TANGO-175 is secreted when expressed in 293T cells. The secretion assay was performed as follows. Approximately 8×10⁵ 293T cells were plated per well in a 6-well plate, and the cells were incubated in growth medium (DMEM, 10% fetal bovine serum, penicillin/strepomycin) at 33° C., 5% CO₂ overnight. The 293T cells were transfected with 2 μg of full-length human TANGO 175 inserted in the pMET7 vector/well and 10 μg LipofectAMINE (GIBCO/BRL Cat. #18324-012)/well according to the protocol for GIBCO/BRL LipofectAMINE. The growth medium was replaced 5 hours later to allow the cells to recover overnight. Next, the medium was removed and each well was gently washed twice with DMEM without methionine and cysteine (ICN Cat. #16-424-54). Next, 1 ml DMEM without methionine and cysteine with 50 μCi Trans-³⁵S (ICN Cat. #51006) was added to each well and the cells were incubated at 33° C., 5% CO₂ for the appropriate time period. A 150 μl aliquot of conditioned medium was obtained and 150 μl of 2×SDS sample buffer was added to the aliquot. The sample was heat-inactivated and loaded on a 4-20% SDS-PAGE gel. The gel was fixed and the presence of secreted protein was detected by autoradiography.

### Example 23

### Preparation of TANGO-0.175 Proteins

Recombinant TANGO-175 can be produced in a variety of expression systems. For example, the mature TANGO-175 peptide can be expressed as a recombinant glutathione-S-transferase (GST) fusion protein in E. coli and the fusion protein can be isolated and characterized. Specifically, as described above, TANGO-175 can be fused to GST and this fusion protein can be expressed in E. coli strain PEB 199. Expression of the GST-TANGO-175 fusion protein in PEB199 can be induced with IPTG. The recombinant fusion protein can be purified from crude bacterial lysates of the induced PEB 199 strain by affinity chromatography on glutathione beads.

### Example 24

### Assaying the Expression of TANGO-175 in a Murine Model of Mice with Septic Shock

To determine whether TANGO-175 is expressed in response to septic shock a mouse model of septic shock was used. Mice were injected intravenously with either 20 mg/kg lipolysaccharide (LPS) or, as a control, PBS, and sacrificed at 2, 8 or 24 hours post-injection. Organs were harvested and cDNA was prepared for use in TaqMan as described above. The level of TANGO-175 gene expression was significantly upregulated in liver, heart and spleen by 8 hours post-LPS compared to PBS controls.

### Example 25

### Measurement of TANGO-175 or WDNM-2 Activity

The ability of a TANGO-175 or WDNM-2 polypeptide or a variant thereof to modulate hematopoiesis can be measured using the assay described by Goselink et al. (J. Exp Med. 184:1305-12, 1996). Alternatively, a colony formation assay can be used. Briefly, a single cell suspension of washed, RBC-free bone marrow cells is obtained as described above and diluted to 5×10⁴ cells/ml in methylcellulose (StemCell Technologies, Inc.). Next, 0.1 ml of diluted bone marrow cells in methylcellulose are added to the wells of a 96-well round bottom tissue culture plate (Corning) containing 11 ul of supernatant. The plates are incubated at 33° C. in a 5% CO₂ humidified chamber for 7 days at which time the number of colonies in each well are counted.

The ability of a TANGO-175 or WDNM-2 polypeptide or a variant thereof to modulate LPS-responsiveness can be measured using the assay described by Jin et al. (Cell 88:417-26, 1997).

Alternatively, the ability to modulate the effect of septic shock in mice is evaluated using the mouse septic shock model. Briefly, the protein being tested is administered to mice prior to or simultaneously with administration of 20 mg/kg LPS or or PBS (which serves as a control). The mice are then sacrificed at 2, 8 or 24 hours post-injection of the mixture. The modulatory effect of TANGO-175 on LPS-induced septic shock in mice is evaluated.

The ability of a TANGO-175 or WDNM-2 polypeptide or variant thereof to inoculate coagulation can be tested using standard assays. Kits for performing coagulation assays are available from American Bioproducts Company (New Jersey) and Helene Laboratories (San Rafeal, Calif.).

Equivalents

Those skilled in the art will recognize, or be able to ascertain using no more than routine experimentation, many equivalents to the specific embodiments of the invention described herein. Such equivalents are intended to be encompassed by the following claims.

1. An isolated nucleic acid molecule selected from the group consisting of:
a) a nucleic acid molecule having a nucleotide sequence which is at least 90% identical to the nucleotide sequence of any of SEQ ID NOs:1, 3, 9, 11, 29, 31, 40, 33, 35, 41, 37, 43, 45, 46, 47, 48, 49, 50, 51, 52, 53, 55, and 57, and the nucleotide sequence of any of the clones deposited as ATCC Accession numbers 98693, 98801, 98802, and 98694, or a complement thereof;
b) a nucleic acid molecule comprising at least 15 nucleotide residues and having a nucleotide sequence identical to at least 15 consecutive nucleotide residues of any of SEQ ID NOs:1, 3, 9, 11, 29, 31, 40, 33, 35, 41, 37, 43, 45, 46, 47, 48, 49, 50, 51, 52, 53, 55, and 57, and the nucleotide sequence of any of the clones deposited as ATCC Accession numbers 98693, 98801, 98802, and 98694, or a complement thereof;
c) a nucleic acid molecule which encodes a polypeptide comprising the amino acid sequence of any of SEQ ID NOs:2, 4, 10, 12, 30, 42, 32, 34, 36, 38, 44, 63, 54, 64, 56, and 67, and the amino acid sequence encoded by the nucleotide sequence of any of the clones deposited as ATCC Accession numbers 98693, 98801, 98802, and 98694;
d) a nucleic acid molecule which encodes a fragment of a polypeptide comprising the amino acid sequence of any of SEQ ID NOs:2, 4, 10, 12, 30, 42, 32, 34, 36, 38, 44, 63, 54, 64, 56, and 67, and the amino acid sequence encoded by the nucleotide sequence of any of the clones deposited as ATCC Accession numbers 98693, 98801, 98802, and 98694, wherein the fragment comprises at least 10 consecutive amino acid residues of any of SEQ ID NOs:2, 4, 10, 12, 30, 42, 32, 34, 36, 38, 44, 63, 54, 64, 56, and 67, and the amino acid sequence encoded by the nucleotide sequence of any of the clones deposited as ATCC Accession numbers 98693, 98801, 98802, and 98694; and
e) a nucleic acid molecule which encodes a fragment of a polypeptide comprising the amino acid sequence of any of SEQ ID NOs:2, 4, 10, 12, 30, 42, 32, 34, 36, 38, 44, 63, 54, 64, 56, and 67, and the amino acid sequence encoded by the nucleotide sequence of any of the clones deposited as ATCC Accession numbers 98693, 98801, 98802, and 98694, wherein the fragment comprises consecutive amino acid residues corresponding to at least half of the full length of any of SEQ ID NOs:2, 4, 10, 12, 30, 42, 32, 34, 36, 38, 44, 63, 54, 64, 56, and 67, and the amino acid sequence encoded by the nucleotide sequence of any of the clones deposited as ATCC Accession numbers 98693, 98801, 98802, and 98694; and
f) a nucleic acid molecule which encodes a naturally occurring allelic variant of a polypeptide comprising the amino acid sequence of any of SEQ ID NOs: 2, 4, 10, 12, 30, 42, 32, 34, 36, 38, 44, 63, 54, 64, 56, and 67, wherein the nucleic acid molecule hybridizes with a nucleic acid molecule consisting of the nucleotide sequence of any of SEQ ID NOs:1, 3, 9, 11, 29, 31, 40, 33, 35, 41, 37, 43, 45, 46, 47, 48, 49, 50, 51, 52, 53, 55, and 57, and the nucleotide sequence of any of the clones deposited as ATCC Accession numbers 98693, 98801, 98802, and 98694, or a complement thereof under stringent conditions.

2. The isolated nucleic acid molecule of claim 1, which is selected from the group consisting of:
a) a nucleic acid having the nucleotide sequence of any of SEQ ID NOs:1, 3, 9, 11, 29, 31, 40, 33, 35, 41, 37, 43, 45, 46, 47, 48, 49, 50, 51, 52, 53, 55, and 57, and the nucleotide sequence of any of the clones deposited as ATCC Accession numbers. 98693, 98801, 98802, and 98694, or a complement thereof; and
b) a nucleic acid molecule which encodes a polypeptide having the amino acid sequence of any of SEQ ID NOs:2, 4, 10, 12, 30, 42, 32, 34, 36, 38, 44, 63, 54, 64, 56, and 67 and the amino acid sequence encoded by the nucleotide sequence of any of the clones deposited as ATCC Accession numbers 98693, 98801, 98802, and 98694, or a complement thereof.

3. The nucleic acid molecule of claim 1, further comprising vector nucleic acid sequences.

4. The nucleic acid molecule of claim 1 further comprising nucleic acid sequences encoding a heterologous polypeptide.

5. A host cell which contains the nucleic acid molecule of claim 1.

6. The host cell of claim 5 which is a mammalian host cell.

7. A non-human mammalian host cell containing the nucleic acid molecule of claim 1.

8. An isolated polypeptide selected from the group consisting of:
a) a fragment of a polypeptide comprising the amino acid sequence of any of SEQ ID NOs:2, 4, 10, 12, 30, 42, 32, 34, 36, 38, 44, 63, 54, 64, 56, and 67 and the amino acid sequence encoded by the nucleotide sequence of any of the clones deposited as ATCC Accession numbers 98693, 98801, 98802, and 98694;
b) a naturally occurring allelic variant of a polypeptide comprising the amino acid sequence of any of SEQ ID NOs:2, 4, 10, 12, 30, 42, 32, 34, 36, 38, 44, 63, 54, 64, 56, and 67, wherein the polypeptide is encoded by a nucleic acid molecule which hybridizes with a nucleic acid molecule consisting of the nucleotide sequence of any of SEQ ID NOs:1, 3, 9, 11, 29, 31, 40, 33, 35, 41, 37, 43, 45, 46, 47, 48, 49, 50, 51, 52, 53, 55, and 57, and the nucleotide sequence of any of the clones deposited as ATCC Accession numbers 98693, 98801, 98802, and 98694, or a complement thereof under stringent conditions; and
c) a polypeptide which is encoded by a nucleic acid molecule comprising a nucleotide sequence which is at least 90% identical to a nucleic acid consisting of the nucleotide sequence of any of SEQ ID NOs:1, 3, 9, 11, 29, 31, 40, 33, 35, 41, 37, 43, 45, 46, 47, 48, 49, 50, 51, 52, 53, 55, and 57, and the nucleotide sequence of any of the clones deposited as ATCC Accession numbers 98693, 98801, 98802, and 98694, or a complement thereof.

9. The isolated polypeptide of claim 8 having the amino acid sequence of any of SEQ ID NOs:2, 4, 10, 12, 30, 42, 32, 34, 36, 38, 44, 63, 54, 64, 56, and 67, and the amino acid sequence encoded by the nucleotide sequence of any of the clones deposited as ATCC Accession numbers 98693, 98801, 98802, and 98694.

10. The polypeptide of claim 8, wherein the amino acid sequence of the polypeptide further comprises heterologous amino acid residues.

11. An antibody which selectively binds with the polypeptide of claim 8.

12. A method for producing a polypeptide selected from the group consisting of:
a) a polypeptide comprising the amino acid sequence of any of SEQ ID NOs:2, 4, 10, 12, 30, 42, 32, 34, 36, 38, 44, 63, 54, 64, 56, and 67 and the amino acid sequence encoded by the nucleotide sequence of any of the clones deposited as ATCC Accession numbers 98693, 98801, 98802, and 98694;
b) a polypeptide comprising a fragment of the amino acid sequence of any of SEQ ID NOs:2, 4, 10, 12, 30, 42, 32, 34, 36, 38, 44, 63, 54, 64, 56, and 67 and the amino acid sequence encoded by the nucleotide sequence of any of the clones deposited as ATCC Accession numbers 98693, 98801, 98802, and 98694, wherein the fragment comprises at least 10 contiguous amino acids of any of SEQ ID NOs:2, 4, 10, 12, 30, 42, 32, 34, 36, 38, 44, 63, 54, 64, 56, and 67 and the amino acid sequence encoded by the nucleotide sequence of any of the clones deposited as ATCC Accession numbers 98693, 98801, 98802, and 98694; and
c) a naturally occurring allelic variant of a polypeptide comprising the amino acid sequence of any of SEQ ID NOs:2, 4, 10, 12, 30, 42, 32, 34, 36, 38, 44, 63, 54, 64, 56, and 67, or a complement thereof, wherein the polypeptide is encoded by a nucleic acid molecule which hybridizes with a nucleic acid molecule consisting of the nucleotide sequence of any of SEQ ID NOs:1, 3, 9, 11, 29, 31, 40, 33, 35, 41, 37, 43, 45, 46, 47, 48, 49, 50, 51, 52, 53, 55, and 57, and the nucleotide sequence of any of the clones deposited as ATCC Accession numbers 98693, 98801, 98802, and 98694, or a complement thereof under stringent conditions;
the method comprising culturing the host cell of claim 5 under conditions in which the nucleic acid molecule is expressed.

13. A method for detecting the presence of a polypeptide of claim 8 in a sample, comprising:
a) contacting the sample with a compound which selectively binds with a polypeptide of claim 8; and
b) determining whether the compound binds with the polypeptide in the sample.

14. The method of claim 13, wherein the compound which binds with the polypeptide is an antibody.

15. A kit comprising a compound which selectively binds with a polypeptide of claim 8 and instructions for use.

16. A method for detecting the presence of a nucleic acid molecule of claim 1 in a sample, comprising the steps of:
a) contacting the sample with a nucleic acid probe or primer which selectively hybridizes with the nucleic acid molecule; and
b) determining whether the nucleic acid probe or primer binds with a nucleic acid molecule in the sample.

17. The method of claim 16, wherein the sample comprises mRNA molecules and is contacted with a nucleic acid probe.

18. A kit comprising a compound which selectively hybridizes with a nucleic acid molecule of claim 1 and instructions for use.

19. A method for identifying a compound which binds with a polypeptide of claim 8 comprising the steps of:
a) contacting a polypeptide, or a cell expressing a polypeptide of claim 8 with a test compound; and
b) determining whether the polypeptide binds with the test compound.

20. The method of claim 19, wherein the binding of the test compound to the polypeptide is detected by a method selected from the group consisting of:
a) detection of binding by direct detecting of test compound/polypeptide binding;
b) detection of binding using a competition binding assay;
c) detection of binding using an assay for an activity characteristic of the polypeptide.

21. A method for modulating the activity of a polypeptide of claim 8 comprising contacting a polypeptide or a cell expressing a polypeptide of claim 8 with a compound which binds with the polypeptide in a sufficient concentration to modulate the activity of the polypeptide.

22. A method for identifying a compound which modulates the activity of a polypeptide of claim 8, comprising:
a) contacting a polypeptide of claim 8 with a test compound; and
b) determining the effect of the test compound on the activity of the polypeptide to thereby identify a compound which modulates the activity of the polypeptide.

23. An antibody substance which selectively binds with the polypeptide of claim 8.

24. A method of making an antibody substance which selectively binds with the polypeptide of claim 8, the method comprising providing the polypeptide to an immunocompetent vertebrate and thereafter harvesting from the vertebrate blood or serum comprising the antibody substance.

25. A method of making an antibody substance which selectively binds with the polypeptide of claim 8, the method comprising contacting the polypeptide with a plurality of particles which individually comprise an antibody substance and a a nucleic acid encoding the antibody substance, segregating a particle which selectively binds with the polypeptide, and expressing the antibody substance from the nucleic acid of the segregated particle.

26-64. (Presently Canceled)


SEQUENCE LISTING (68 sequences)

SEQ ID NO: 1 - DNA, 1856 bp, Homo sapiens
  CDS (95)...(1432)
gtcgacccacgcgtccggctccatccagcctgagaaacaagccgggtggctgagccaggctgtgcacggagtgcctgacgggcccaacagacccatgctgcatccagagacctcccctggccgggggcatctcctggctgtgctcctggccctccttggcaccgcctgggcagaggtgtggccaccccagctgcaggagcaggctccgatggccggagccctgaacaggaaggagagtttcttgctcctctccctgcacaaccgcctgcgcagctgggtccagccccctgcggctgacatgcggaggctggactggagtgacagcctggcccaactggctcaagccagggcagccctctgtggaatcccaaccccgagcctggcgtccggcctgtggcgcaccctgcaagtgggctggaacatgcagctgctgcccgcgggcttggcgtcctttgttgaagtggtcagcctatggtttgcagaggggcagcggtacagccacgcggcaggagagtgtgctcgcaacgccacctgcacccactacacgcagctcgtgtgggccacctcaagccagctgggctgtgggcggcacctgtgctctgcaggccaggcagcgatagaagcctttgtctgtgcctactcccccggaggcaactgggaggtcaacgggaagacaatcatcccctataagaagggtgcctggtgttcgctctgcacagccagtgtctcaggctgcttcaaagcctgggaccatgcaggggggctctgtgaggtccccaggaatccttgtcgcatgagctgccagaaccatggacgtctcaacatcagcacctgccactgccactgtccccctggctacacgggcagatactgccaagtgaggtgcagcctgcagtgtgtgcacggccggttccgggaggaggagtgctcgtgcgtctgtgacatcggctacgggggagcccagtgtgccaccaaggtgcattttcccttccacacctgtgacctgaggatcgacggagactgcttcatggtgtcttcagaggcagacacctattacagagccaggatgaaatgtcagaggaaaggcggggtgctggcccagatcaagagccagaaagtgcaggacatcctcgccttctatctgggccgcctggagaccaccaacgaggtgattgacagtgacttcgagaccaggaacttctggatcgggctcacctacaagaccgccaaggactccttccgctgggccacaggggagcaccaggccttcaccagttttgcctttgggcagcctgacaaccacgggtttggcaactgcgtggagctgcaggcttcagctgccttcaactggaacaaccagcgctgcaaaacccgaaaccgttacatctgccagtttgcccaggagcacatctcccggtggggcccagggtcctgaggcctgaccacatggctccctcgcctgccctgggagcaccggctctgcttacctgtccgcccacctgtctggaacaagggccaggttaagaccacatgcctcatgtccaaagaggtctcagaccttgcacaatgccagaagttgggcagagagaggcagggaggccagtgagggccagggagtgagtgttagaagaagctggggcccttcgcctgcttttgattgggaagatgggcttcaattagatggcaaaggagaggacaccgccagtggtccaaaaaggctgctctcttccacctggcccagaccctgtggggcagcggagcttccctgtggcatgaaccccacagggtattaaattatgaatcagctgaaaaaaaaaaaaaaaaaaaaaaaaaaaaaaaaaaaaaagggcggccgc

SEQ ID NO: 2 - protein, 446 aa, Homo sapiens [sequence omitted]

SEQ ID NO: 3 - DNA, 1338 bp, Homo sapiens
atgctgcatccagagacctcccctggccgggggcatctcctggctgtgctcctggccctccttggcaccgcctgggcagaggtgtggccaccccagctgcaggagcaggctccgatggccggagccctgaacaggaaggagagtttcttgctcctctccctgcacaaccgcctgcgcagctgggtccagccccctgcggctgacatgcggaggctggactggagtgacagcctggcccaactggctcaagccagggcagccctctgtggaatcccaaccccgagcctggcgtccggcctgtggcgcaccctgcaagtgggctggaacatgcagctgctgcccgcgggcttggcgtcctttgttgaagtggtcagcctatggtttgcagaggggcagcggtacagccacgcggcaggagagtgtgctcgcaacgccacctgcacccactacacgcagctcgtgtgggccacctcaagccagctgggctgtgggcggcacctgtgctctgcaggccaggcagcgatagaagcctttgtctgtgcctactcccccggaggcaactgggaggtcaacgggaagacaatcatcccctataagaagggtgcctggtgttcgctctgcacagccagtgtctcaggctgcttcaaagcctgggaccatgcaggggggctctgtgaggtccccaggaatccttgtcgcatgagctgccagaaccatggacgtctcaacatcagcacctgccactgccactgtccccctggctacacgggcagatactgccaagtgaggtgcagcctgcagtgtgtgcacggccggttccgggaggaggagtgctcgtgcgtctgtgacatcggctacgggggagcccagtgtgccaccaaggtgcattttcccttccacacctgtgacctgaggatcgacggagactgcttcatggtgtcttcagaggcagacacctattacagagccaggatgaaatgtcagaggaaaggcggggtgctggcccagatcaagagccagaaagtgcaggacatcctcgccttctatctgggccgcctggagaccaccaacgaggtgattgacagtgacttcgagaccaggaacttctggatcgggctcacctacaagaccgccaaggactccttccgctgggccacaggggagcaccaggccttcaccagttttgcctttgggcagcctgacaaccacgggtttggcaactgcgtggagctgcaggcttcagctgccttcaactggaacaaccagcgctgcaaaacccgaaaccgttacatctgccagtttgcccaggagcacatctcccggtggggcccagggtcc

SEQ ID NO: 4 - protein, 420 aa, Homo sapiens
EVWPPQLQEQAPMAGALNRKESFLLLSLHNRLRSWVQPPAADMRRLDWSDSLAQLAQARAALCGIPTPSLASGLWRTLQVGWNMQLLPAGLASFVEVVSLWFAEGQRYSHAAGECARNATCTHYTQLVWATSSQLGCGRHLCSAGQAAIEAFVCAYSPGGNWEVNGKTIIPYKKGAWCSLCTASVSGCFKAWDHAGGLCEVPRNPCRMSCQNHGRLNISTCHCHCPPGYTGRYCQVRCSLQCVHGRFREEECSCVCDIGYGGAQCATKVHFPFHTCDLRIDGDCFMVSSEADTYYRARMKCQRKGGVLAQIKSQKVQDILAFYLGRLETTNEVIDSDFETRNFWIGLTYKTAKDSFRWATGEHQAFTSFAFGQPDNHGFGNCVELQASAAFNWNNQRCKTRNRYICQFAQEHISRWGPGS

SEQ ID NO: 5 - DNA, 30 bp, Artificial Sequence
  oligonucleotide for PCR
ctctggatgcagcatgggtctgttgggccc

SEQ ID NO: 6 - DNA, 20 bp, Artificial Sequence
  oligonucleotide for PCR
gatgcagcatgggtctgttg

SEQ ID NO: 7 - DNA, 17 bp, Artificial Sequence
  oligonucleotide for PCR
ccatgctgcatccagag

SEQ ID NO: 8 - DNA, 20 bp, Artificial Sequence
  oligonucleotide for PCR
cacagacaaaggcttctatc

SEQ ID NO: 9 - DNA, 1512 bp, Homo sapiens
  CDS (274)...(1092)
gtcgacccacgcgtccgctcaggaggtgcctccaggcggccagtgggcctgaggccccagcaagggctagggtccatctccagtcccaggacacagcagcggccaccatggccacgcctgggctccagcagcatcagcagcccccaggaccggggaggcacaggtggcccccaccacccggaggagcagctcctgcccctgtccgggggatgactgattctcctccgccaggccacccagaggagaaggccaccccgcctggaggcacaggccatgaggggctctcaggaggtgctgctgatgtggcttctggtgttggcagtgggcggcacagagcacgcctaccggcccggccgtagggtgtgtgctgtccgggctcacggggaccctgtctccgagtcgttcgtgcagcgtgtgtaccagcccttcctcaccacctgcgacgggcaccgggcctgcagcacctaccgaaccatctataggaccgcctaccgccgcagccctgggctggcccctgccaggcctcgctacgcgtgctgccccggctggaagaggaccagcgggcttcctggggcctgtggagcagcaatatgccagccgccatgccggaacggagggagctgtgtccagcctggccgctgccgctgccctgcaggatggcggggtgacacttgccagtcagatgtggatgaatgcagtgctaggaggggcggctgtccccagcgctgcatcaacaccgccggcagttactggtgccagtgttgggaggggcacagcctgtctgcagacggtacactctgtgtgcccaagggagggccccccagggtggcccccaacccgacaggagtggacagtgcaatgaaggaagaagtgcagaggctgcagtccagggtggacctgctggaggagaagctgcagctggtgctggccccactgcacagcctggcctcgcaggcactggagcatgggctcccggaccccggcagcctcctggtgcactccttccagcagctcggccgcatcgactccctgagcgagcagatttccttcctggaggagcagctggggtcctgctcctgcaagaaagactcgtgactgcccagcgccccaggctggactgagcccctcacgccgccctgcagcccccatgcccctgcccaacatgctgggggtccagaagccacctcggggtgactgagcggaaggccaggcagggccttcctcctcttcctcctccccttcctcgggaggctccccagaccctggcatgggatgggctgggatcttctctgtgaatccacccctggctacccccaccctggctaccccaacggcatcccaaggccaggtgggccctcagctgagggaaggtacgagctccctgctggagcctgggacccatggcacaggccaggcagcccggaggctgggtggggcctcagtgggggctgctgcctgacccccagcacaataaaaatgaaacgtgaaaaaaaaaaaaaaaagggcggccgc

SEQ ID NO: 10 - protein, 273 aa, Homo sapiens [sequence omitted]

SEQ ID NO: 11 - DNA, 819 bp, Homo sapiens
atgaggggctctcaggaggtgctgctgatgtggcttctggtgttggcagtgggcggcacagagcacgcctaccggcccggccgtagggtgtgtgctgtccgggctcacggggaccctgtctccgagtcgttcgtgcagcgtgtgtaccagcccttcctcaccacctgcgacgggcaccgggcctgcagcacctaccgaaccatctataggaccgcctaccgccgcagccctgggctggcccctgccaggcctcgctacgcgtgctgccccggctggaagaggaccagcgggcttcctggggcctgtggagcagcaatatgccagccgccatgccggaacggagggagctgtgtccagcctggccgctgccgctgccctgcaggatggcggggtgacacttgccagtcagatgtggatgaatgcagtgctaggaggggcggctgtccccagcgctgcatcaacaccgccggcagttactggtgccagtgttgggaggggcacagcctgtctgcagacggtacactctgtgtgcccaagggagggccccccagggtggcccccaacccgacaggagtggacagtgcaatgaaggaagaagtgcagaggctgcagtccagggtggacctgctggaggagaagctgcagctggtgctggccccactgcacagcctggcctcgcaggcactggagcatgggctcccggaccccggcagcctcctggtgcactccttccagcagctcggccgcatcgactccctgagcgagcagatttccttcctggaggagcagctggggtcctgctcctgcaagaaagactcg

SEQ ID NO: 12 - protein, 251 aa, Homo sapiens
AYRPGRRVCAVRAHGDPVSESFVQRVYQPFLTTCDGHRACSTYRTIYRTAYRRSPGLAPARPRYACCPGWKRTSGLPGACGAAICQPPCRNGGSCVQPGRCRCPAGWRGDTCQSDVDECSARRGGCPQRCINTAGSYWCQCWEGHSLSADGTLCVPKGGPPRVAPNPTGVDSAMKEEVQRLQSRVDLLEEKLQLVLAPLHSLASQALEHGLPDPGSLLVHSFQQLGRIDSLSEQISFLEEQLGSCSCKKDS

SEQ ID NO: 13 - DNA, 846 bp, Mus musculus
  CDS (13)...(837)
ggtaccgccaccatgtggggctccggagaactgcttgtagcatggtttctagtgttggcagcagatggtactactgagcatgtctacagacccagccgtagagtgtgtactgtggggatttccggaggttccatctcggagacctttgtgcagcgtgtataccagccttacctcaccacttgcgacggacacagagcctgcagcacctaccgaaccatctaccggactgcctatcgccgtagccctggggtgactcccgcaaggcctcgctatgcttgctgccctggttggaagaggaccagtgggctccctggggcttgtggagcagcaatatgccagcctccatgtgggaatggagggagttgcatccgcccaggacactgccgctgccctgtgggatggcagggagatacttgccagacagatgttgatgaatgcagtacaggagaggccagttgtccccagcgctgtgtcaatactgtgggaagttactggtgccagggatgggagggacaaagcccatctgcagatgggacgcgctgcctgtctaaggaggggccctccccggtggccccaaaccccacagcaggagtggacagcatggcgagagaggaggtgtacaggctgcaggctcgggttgatgtgctagaacagaaactgcagttggtgctggccccactgcacagcctggcctctcggtccacagagcatgggctacaagatcctggcagcctgctggcccattccttccagcagctggaccgaattgattcactgagtgagcaggtgtccttcttggaggaacatctggggtcctgctcctgcaaaaaagatctgtgactcgag

SEQ ID NO: 14 - DNA, 825 bp, Mus musculus
atgtggggctccggagaactgcttgtagcatggtttctagtgttggcagcagatggtactactgagcatgtctacagacccagccgtagagtgtgtactgtggggatttccggaggttccatctcggagacctttgtgcagcgtgtataccagccttacctcaccacttgcgacggacacagagcctgcagcacctaccgaaccatctaccggactgcctatcgccgtagccctggggtgactcccgcaaggcctcgctatgcttgctgccctggttggaagaggaccagtgggctccctggggcttgtggagcagcaatatgccagcctccatgtgggaatggagggagttgcatccgcccaggacactgccgctgccctgtgggatggcagggagatacttgccagacagatgttgatgaatgcagtacaggagaggccagttgtccccagcgctgtgtcaatactgtgggaagttactggtgccagggatgggagggacaaagcccatctgcagatgggacgcgctgcctgtctaaggaggggccctccccggtggccccaaaccccacagcaggagtggacagcatggcgagagaggaggtgtacaggctgcaggctcgggttgatgtgctagaacagaaactgcagttggtgctggccccactgcacagcctggcctctcggtccacagagcatgggctacaagatcctggcagcctgctggcccattccttccagcagctggaccgaattgattcactgagtgagcaggtgtccttcttggaggaacatctggggtcctgctcctgcaaaaaagatctg

SEQ ID NO: 15 - protein, 275 aa, Mus musculus
MWGSGELLVAWFLVLAADGTTEHVYRPSRRVCTVGISGGSISETFVQRVYQPYLTTCDGHRACSTYRTIYRTAYRRSPGVTPARPRYACCPGWKRTSGLPGACGAAICQPPCGNGGSCIRPGHCRCPVGWQGDTCQTDVDECSTGEASCPQRCVNTVGSYWCQGWEGQSPSADGTRCLSKEGPSPVAPNPTAGVDSMAREEVYRLQARVDVLEQKLQLVLAPLHSLASRSTEHGLQDPGSLLAHSFQQLDRIDSLSEQVSFLEEHLGSCSCKKDL

SEQ ID NO: 16 - DNA, 1475 bp, Homo sapiens
  CDS (194)...(442)
ttaggatcgaccccgcgtctgcggacgcgtgggcggacgcgtccgcaagctggccctgcacggctgcaagggaggctcctgtggacaggccaggcaggtgggcctcaggaggtgcctccaggcggccagtgggcctgaggccccagcaagggctagggtccatctccagtcccaggacacagcagcggccaccatggccacgcctgggctccagcagcatcagcagcccccaggaccggggaggcacaggtggcccccaccacccggaggagcagctcctgcccctgtccgggggatgactgattctcctccgccaggccacccagaggagaaggccaccccgcctggaggcacaggccatgaggggctctcaggaggtgctgctgatgtggcttctggtgttggcagtgggcggcacagagcacgcctaccggcccggccgtagggtgtgtgctgtccgggctcacggggaccctgtctccgagtcgttcgtgcagcgtgtgtaccagcccttcctcaccacctgcgacgggcaccgggcctgcagcacctaccggccagccgccatgccggaacggagggagctgtgtccagcctggccgctgccgctgccctgcaggatggcggggtgacacttgccagtcagatgtggatgaatgcagtgctaggaggggcggctgtccccagcgctgcatcaacaccgccggcagttactggtgccagtgttgggaggggcacagcctgtctgcagacggtacactctgtgtgcccaagggagggccccccagggtggcccccaacccgacaggagtggacagtgcaatgaaggaagaagtgcagaggctgcagtccagggtggacctgctggaggagaagctgcagctggtgctggccccactgcacagcctggcctcgcaggcactggagcatgggctcccggaccccggcagcctcctggtgcactccttccagcagctcggccgcatcgactccctgagcgagcagatttccttcctggaggagcagctggggtcctgctcctgcaagaaagactcgtgactgcccagcgccccaggctggactgagcccctcacgccgccctgcagcccccatgcccctgcccaacatgctgggggtccagaagccacctcggggtgactgagcggaaggccaggcagggccttcctcctcttcctcctccccttcctcgggaggctccccagaccctggcatgggatgggctgggatcttctctgtgaatccacccctggctacccccaccctggctaccccaacggcatcccaaggccaggtgggccctcagctgagggaaggtacgagctccctgctggagcctgggacccatggcacaggccaggcagcccggaggctgggtggggcctcagtgggggctgctgcctgacccccagcacaataaaaatgaaacgtgaaaaaaaaaaaaaaaagggcggccg

SEQ ID NO: 17 - protein, 83 aa, Homo sapiens
MATPGLQQHQQPPGPGRHRWPPPPGGAAPAPVRGMTDSPPPGHPEEKATPPGGTGHEGLSGGAADVASGVGSGRHRARLPARP

SEQ ID NO: 18 - DNA, 249 bp, Homo sapiens
atggccacgcctgggctccagcagcatcagcagcccccaggaccggggaggcacaggtggcccccaccacccggaggagcagctcctgcccctgtccgggggatgactgattctcctccgccaggccacccagaggagaaggccaccccgcctggaggcacaggccatgaggggctctcaggaggtgctgctgatgtggcttctggtgttggcagtgggcggcacagagcacgcctaccggcccggccg

SEQ ID NO: 19 - DNA, 1353 bp, Homo sapiens
  CDS (194)...(934)
ttaggatcgaccccgcgtctgcggacgcgtgggcggacgcgtccgcaagctggccctgcacggctgcaagggaggctcctgtggacaggccaggcaggtgggcctcaggaggtgcctccaggcggccagtgggcctgaggccccagcaagggctagggtccatctccagtcccaggacacagcagcggccaccatggccacgcctgggctccagcagcatcagcagcccccaggaccggggaggcacaggtggcccccaccacccggaggagcagctcctgcccctgtccgggggatgactgattctcctccgccagccgtagggtgtgtgctgtccgggctcacggggaccctgtctccgagtcgttcgtgcagcgtgtgtaccagcccttcctcaccacctgcgacgggcaccgggcctgcagcacctaccggccagccgccatgccggaacggagggagctgtgtccagcctggccgctgccgctgccctgcaggatggcggggtgacacttgccagtcagatgtggatgaatgcagtgctaggaggggcggctgtccccagcgctgcatcaacaccgccggcagttactggtgccagtgttgggaggggcacagcctgtctgcagacggtacactctgtgtgcccaagggagggccccccagggtggcccccaacccgacaggagtggacagtgcaatgaaggaagaagtgcagaggctgcagtccagggtggacctgctggaggagaagctgcagctggtgctggccccactgcacagcctggcctcgcaggcactggagcatgggctcccggaccccggcagcctcctggtgcactccttccagcagctcggccgcatcgactccctgagcgagcagatttccttcctggaggagcagctggggtcctgctcctgcaagaaagactcgtgactgcccagcgccccaggctggactgagcccctcacgccgccctgcagcccccatgcccctgcccaacatgctgggggtccagaagccacctcggggtgactgagcggaaggccaggcagggccttcctcctcttcctcctccccttcctcgggaggctccccagaccctggcatgggatgggctgggatcttctctgtgaatccacccctggctacccccaccctggctaccccaacggcatcccaaggccaggtgggccctcagctgagggaaggtacgagctccctgctggagcctgggacccatggcacaggccaggcagcccggaggctgggtggggcctcagtgggggctgctgcctgacccccagcacaataaaaatgaaacgtgaaaaaaaaaaaaaaaagggcggccg

SEQ ID NO: 20 - protein, 247 aa, Homo sapiens
MATPGLQQHQQPPGPGRHRWPPPPGGAAPAPVRGMTDSPPPAVGCVLSGLTGTLSPSRSCSVCTSPSSPPATGTGPAAPTGQPPCRNGGSCVQPGRCRCPAGWRGDTCQSDVDECSARRGGCPQRCINTAGSYWCQCWEGHSLSADGTLCVPKGGPPRVAPNPTGVDSAMKEEVQRLQSRVDLLEEKLQLVLAPLHSLASQALEHGLPDPGSLLVHSFQQLGRIDSLSEQISFLEEQLGSCSCKKDS

SEQ ID NO: 21 - DNA, 741 bp, Homo sapiens
atggccacgcctgggctccagcagcatcagcagcccccaggaccggggaggcacaggtggcccccaccacccggaggagcagctcctgcccctgtccgggggatgactgattctcctccgccagccgtagggtgtgtgctgtccgggctcacggggaccctgtctccgagtcgttcgtgcagcgtgtgtaccagcccttcctcaccacctgcgacgggcaccgggcctgcagcacctaccggccagccgccatgccggaacggagggagctgtgtccagcctggccgctgccgctgccctgcaggatggcggggtgacacttgccagtcagatgtggatgaatgcagtgctaggaggggcggctgtccccagcgctgcatcaacaccgccggcagttactggtgccagtgttgggaggggcacagcctgtctgcagacggtacactctgtgtgcccaagggagggccccccagggtggcccccaacccgacaggagtggacagtgcaatgaaggaagaagtgcagaggctgcagtccagggtggacctgctggaggagaagctgcagctggtgctggccccactgcacagcctggcctcgcaggcactggagcatgggctcccggaccccggcagcctcctggtgcactccttccagcagctcggccgcatcgactccctgagcgagcagatttccttcctggaggagcagctggggtcctgctcctgcaagaaagactcg

SEQ ID NO: 22 - DNA, 1475 bp, Homo sapiens
  CDS (194)...(823)
ttaggatcgaccccgcgtctgcggacgcgtgggcggacgcgtccgcaagctggccctgcacggctgcaagggaggctcctgtggacaggccaggcaggtgggcctcaggaggtgcctccaggcggccagtgggcctgaggccccagcaagggctagggtccatctccagtcccaggacacagcagcggccaccatggccacgcctgggctccagcagcatcagcagcccccaggaccggggaggcacaggtggcccccaccacccggaggagcagctcctgcccctgtccgggggatgactgattctcctccgccagccgtagggtgtgtgctgtccgggctcacggggaccctgtctccgagtcgttcgtgcagcgtgtgtaccagcccttcctcaccacctgcgacgggcaccgggcctgcagcacctaccgaaccatctataggaccgcctaccgccgcagccctgggctggcccctgccaggcctcgctacgcgtgctgccccggctggaagaggaccagcgggcttcctggggcctgtggagcagcaatatgccagccgccatgccggaacggagggagctgtgtccagcctggccgctgccgctgccctgcaggatggcggggtgacacttgccagtcagatgtggatgaatgcagtgctaggaggggcggctgtccccagcgctgcatcaacaccgccggcagttactggtgccagtgttgggaggggcacagcctgtctgcagacggtacactctgtgtgcccaagggagggccccccagggtggcccccaacccgacaggagtggacagtgcaatgaaggaagaagtgcagaggctgcagtccagggtggacctgctggaggagaagctgcagctggtgctggccccactgcacagcctggcctcgcaggcactggagcatgggctcccggaccccggcagcctcctggtgcactccttccagcagctcggccgcatcgactccctgagcgagcagatttccttcctggaggagcagctggggtcctgctcctgcaagaaagactcgtgactgcccagcgccccaggctggactgagcccctcacgccgccctgcagcccccatgcccctgcccaacatgctgggggtccagaagccacctcggggtgactgagcggaaggccaggcagggccttcctcctcttcctcctccccttcctcgggaggctccccagaccctggcatgggatgggctgggatcttctctgtgaatccacccctggctacccccaccctggctaccccaacggcatcccaaggccaggtgggccctcagctgagggaaggtacgagctccctgctggagcctgggacccatggcacaggccaggcagcccggaggctgggtggggcctcagtgggggctgctgcctgacccccagcacaataaaaatgaaacgtgaaaaaaaaaaaaaaaagggcggccg

SEQ ID NO: 23 - protein, 210 aa, Homo sapiens
MATPGLQQHQQPPGPGRHRWPPPPGGAAPAPVRGMTDSPPPAVGCVLSGLTGTLSPSRSCSVCTSPSSPPATGTGPAAPTEPSIGPPTAAALGWPLPGLATRAAPAGRGPAGFLGPVEQQYASRHAGTEGAVSSLAAAAALQDGGVTLASQMWMNAVLGGAAVPSAASTPPAVTGASVGRGTACLQTVHSVCPREGPPGWPPTRQEWTVQ

SEQ ID NO: 24 - DNA, 630 bp, Homo sapiens
atggccacgcctgggctccagcagcatcagcagcccccaggaccggggaggcacaggtggcccccaccacccggaggagcagctcctgcccctgtccgggggatgactgattctcctccgccagccgtagggtgtgtgctgtccgggctcacggggaccctgtctccgagtcgttcgtgcagcgtgtgtaccagcccttcctcaccacctgcgacgggcaccgggcctgcagcacctaccgaaccatctataggaccgcctaccgccgcagccctgggctggcccctgccaggcctcgctacgcgtgctgccccggctggaagaggaccagcgggcttcctggggcctgtggagcagcaatatgccagccgccatgccggaacggagggagctgtgtccagcctggccgctgccgctgccctgcaggatggcggggtgacacttgccagtcagatgtggatgaatgcagtgctaggaggggcggctgtccccagcgctgcatcaacaccgccggcagttactggtgccagtgttgggaggggcacagcctgtctgcagacggtacactctgtgtgcccaagggagggccccccagggtggcccccaacccgacaggagtggacagtgcaa

SEQ ID NO: 25 - DNA, 30 bp, Artificial Sequence
  oligonucleotide for PCR
ctgagagcccctcatggcctgtgcctccag

SEQ ID NO: 26 - DNA, 20 bp, Artificial Sequence
  oligonucleotide for PCR
agcccctcatggcctgtgcc

SEQ ID NO: 27 - DNA, 18 bp, Artificial Sequence
  oligonucleotide for PCR
gctcacggggaccctgtc

SEQ ID NO: 28 - DNA, 18 bp, Artificial Sequence
  oligonucleotide for PCR
cagtgcctgcgaggccag

SEQ ID NO: 29 - DNA, 2401 bp, Homo sapiens
  CDS (131)...(1441)
gcggccgcgatggggccgaagcgcccgaagccccggagcccacaaactgccgggcccgcctcgccgccgggacccgggtgcctgggctcggcttgaagcggcggcggcgcaccggcacagccgcgggagcatgggcaggaggatgcggggcgccgccgccaccgcggggctctggctgctggcgctgggctcgctgctggcgctgtggggagggctcctgccgccgcggaccgagctgcccgcctcccggccgcccgaagaccgactcccacggcgcccggcccggagcggcggccccgcgcccgcgcctcgcttccctctgcccccgcccctggcgtgggacgcccgcggcggctccctgaaaactttccgggcgctgctcaccctggcggccggcgcggacggcccgccccggcagtcccggagcgagcccaggtggcacgtgtcagccaggcagccccggccggaggagagcgccgcggtgcacgggggcgtcttctggagccgcggcctggaggagcaggtgcccccgggcttttcggaggcccaggcggcggcgtggctggaggcggctcgcggcgcccggatggtggccctggagcgcgggggttgcgggcgcagctccaaccgactggcccgttttgccgacggcacccgcgcctgcgtgcgctacggcatcaacccggagcagattcagggcgaggccctgtcttactatctggcgcgcctgctgggcctccagcgccacgtgccgccgctggcactggctcgggtggaggctcggggcgcgcagtgggcgcaggtgcaggaggagctgcgcgctgcgcactggaccgagggcagcgtggtgagcctgacacgctggctgcccaacctcacggacgtggtggtgcccgcgccctggcgctcggaggacggccgtctgcgccccctccgggatgccgggggtgagctggccaacctcagccaggcggagctggtggacctagtacaatggaccgacttaatccttttcgactacctgacggccaacttcgaccggctcgtaagcaacctcttcagcctgcagtgggacccgcgcgtcatgcagcgtgccaccagcaacctgcaccgcggtccgggcggggcgctggtctttctggacaatgaggcgggcttggtgcacggctaccgggtagcaggcatgtgggacaagtataacgagccgctgttgcagtcagtgtgcgtgttccgcgagcggaccgcgcggcgcgtcctggagctgcaccgcggacaggacgccgcggcccggctgctgcgcctctaccggcgccacgagcctcgcttccccgagctggccgcccttgcagacccccacgctcagctgctacagcgccgcctcgacttcctcgccaagcacattttgcactgtaaggccaagtacggccgccggtctgggacttagtgtcaccgggaggaaaagagagagatctggggctggggtatggatgatggggggaagggcggtcgcctctgccactgtcagggaccagccggccaacgcccacccgcaaaggtgtctaaaaacttcagcttttcacccacctgcccctttctttcaatcccacgctggttcctttcaaagttctgggaggacgaacctcaccgaggcgagaagtgtaacattctctccacccagcttataaaaggattctttactgtgccagcacggggattggatccgaagaaactggctactggggtttggcccccgagtggccgtccctgtgggagatgcaccccattcttgggcccccctcattccctttccgaaaaaggaaaacttgcgtttgagccgttgagctaattctgcaattttctaccaaacagagcgctggtggccccggagcagggctgtgacattggctggtggagcccccttcctgtgttctccctttgttccagcgccgcgatggtgagatcactgttccaagcagggggacggctcgcgataggacaaagagagcaggacctccagactctggggagccctgcagaccttgacaatttgcctgactcattcctgacctcttgtcattttggcctgaaggctacaaattcagggtcagctgtatgcactaagtcaaataatgaatttcttcctccctctcgcaaccgaccaaaattttgacaacgatgatgttcaccagaaggaaaaaaaaatcagttttatgcactttattttgttttgattttcattttttattaagaaaaaattttattttacagaatttaccttctctgtatatatgtgcataaagtgtggtgtaaatatactaaacaaacttatatttcaataaaagggagtttaaaatttaaaaaaaaaaaaaaaaaaaaaaaaaaaacggacgcgtgggtcgac

SEQ ID NO: 30 - protein, 437 aa, Homo sapiens
  SIGNAL (1)...(28)
MGRRMRGAAATAGLWLLALGSLLALWGGLLPPRTELPASRPPEDRLPRRPARSGGPAPAPRFPLPPPLAWDARGGSLKTFRALLTLAAGADGPPRQSRSEPRWHVSARQPRPEESAAVHGGVFWSRGLEEQVPPGFSEAQAAAWLEAARGARMVALERGGCGRSSNRLARFADGTRACVRYGINPEQIQGEALSYYLARLLGLQRHVPPLALARVEARGAQWAQVQEELRAAHWTEGSVVSLTRWLPNLTDVVVPAPWRSEDGRLRPLRDAGGELANLSQAELVDLVQWTDLILFDYLTANFDRLVSNLFSLQWDPRVMQRATSNLHRGPGGALVFLDNEAGLVHGYRVAGMWDKYNEPLLQSVCVFRERTARRVLELHRGQDAAARLLRLYRRHEPRFPELAALADPHAQLLQRRLDFLAKHILHCKAKYGRRSGT

SEQ ID NO: 31 - DNA, 1311 bp, Homo sapiens
atgggcaggaggatgcggggcgccgccgccaccgcggggctctggctgctggcgctgggctcgctgctggcgctgtggggagggctcctgccgccgcggaccgagctgcccgcctcccggccgcccgaagaccgactcccacggcgcccggcccggagcggcggccccgcgcccgcgcctcgcttccctctgcccccgcccctggcgtgggacgcccgcggcggctccctgaaaactttccgggcgctgctcaccctggcggccggcgcggacggcccgccccggcagtcccggagcgagcccaggtggcacgtgtcagccaggcagccccggccggaggagagcgccgcggtgcacgggggcgtcttctggagccgcggcctggaggagcaggtgcccccgggcttttcggaggcccaggcggcggcgtggctggaggcggctcgcggcgcccggatggtggccctggagcgcgggggttgcgggcgcagctccaaccgactggcccgttttgccgacggcacccgcgcctgcgtgcgctacggcatcaacccggagcagattcagggcgaggccctgtcttactatctggcgcgcctgctgggcctccagcgccacgtgccgccgctggcactggctcgggtggaggctcggggcgcgcagtgggcgcaggtgcaggaggagctgcgcgctgcgcactggaccgagggcagcgtggtgagcctgacacgctggctgcccaacctcacggacgtggtggtgcccgcgccctggcgctcggaggacggccgtctgcgccccctccgggatgccgggggtgagctggccaacctcagccaggcggagctggtggacctagtacaatggaccgacttaatccttttcgactacctgacggccaacttcgaccggctcgtaagcaacctcttcagcctgcagtgggacccgcgcgtcatgcagcgtgccaccagcaacctgcaccgcggtccgggcggggcgctggtctttctggacaatgaggcgggcttggtgcacggctaccgggtagcaggcatgtgggacaagtataacgagccgctgttgcagtcagtgtgcgtgttccgcgagcggaccgcgcggcgcgtcctggagctgcaccgcggacaggacgccgcggcccggctgctgcgcctctaccggcgccacgagcctcgcttccccgagctggccgcccttgcagacccccacgctcagctgctacagcgccgcctcgacttcctcgccaagcacattttgcactgtaaggccaagtacggccgccggtctgggact

SEQ ID NO: 32 - protein, 480 aa, Homo sapiens
RPRWGRSARSPGAHKLPGPPRRRDPGAWARLEAAAAHRHSRGSMGRRMRGAAATAGLWLLALGSLLALWGGLLPPRTELPASRPPEDRLPRRPARSGGPAPAPRFPLPPPLAWDARGGSLKTFRALLTLAAGADGPPRQSRSEPRWHVSARQPRPEESAAVHGGVFWSRGLEEQVPPGFSEAQAAAWLEAARGARMVALERGGCGRSSNRLARFADGTRACVRYGINPEQIQGEALSYYLARLLGLQRHVPPLALARVEARGAQWAQVQEELRAAHWTEGSVVSLTRWLPNLTDVVVPAPWRSEDGRLRPLRDAGGELANLSQAELVDLVQWTDLILFDYLTANFDRLVSNLFSLQWDPRVMQRATSNLHRGPGGALVFLDNEAGLVHGYRVAGMWDKYNEPLLQSVCVFRERTARRVLELHRGQDAAARLLRLYRRHEPRFPELAALADPHAQLLQRRLDFLAKHILHCKAKYGRRSGT

SEQ ID NO: 33 - DNA, 2148 bp, Mus musculus
  CDS (103)...(1452)
gccccggagccagctagcagccgggcccgcctcgccgcccgcacccgggcgcccgggcttggcttgaagcggcggcggtggcaccggcgcgccggcaggagcatggggaggaagatgcggggcgccgccgccgccgcggggctctggctgctggctttgagctcgctgctgacgctgtggggaggactcctgccaccgcggaccgagctgccagcctcccggccgcccgaagatcgactccctccgcatccgatccagagtggcggccccgcgcccgagccgcgattccctctgcccccgcccctagtatgggacgcccgcggcggctccctgaaaactttccgggcgctgctcaccctggcggccggcgcggataacccgcctaggaggcaccaggacgaccgcgggcggcacgagccctccgggctgtcctggccagaggagcgcagggcggtgcacgggggcgtcttctggagccgcggcctggaggagcaggttccccggggcttttccgaagcccaagcagcagcgtggctggaggtggcacggggtgctcgggtggtggctctggatcgcgggggctgcggacgcagttccaaccgcctagcccgctttgccgacggcacccgtgcctgtgtacgctacggcatcaacccagagcagatacagggcgaggccctgtcctactaccttgcgcgcctgctgggcctccagcgccacgtgccgccgctggcactggctcgggtggaggctcggggcgcgcagtgggtgcaggtgcaggaggagctgcgcaccgcgcactggaccgagggcagcgtggtgagcctgacgcgctggctgcctaacctcaccgacgtggtggtgcccgagccctggcgatcagaggacggccgtctgcggcccctgcgcgacgccgggggcgagctgaccaacctcagccaggcggagctggtggacttggtacaatggaccgatctgatcctcttcgattacctgacggccaactttgaccggcttgtgagcaacctcttcagcttacagtgggacccacgcgttatgcaccgcgctacgagcaacctgcaccgaggaccaggaggggcgttggtctttctggacaatgaggcgggcttggtgcacggctaccgggtagccggcatgtgggacaagtataacgaaccgctgctacagtcggtgtgtgtattccgagagcggactgctaggcgcgtcttggagctgcaccggggtcaggacgcggcggcccggttgctgcgcctctacagtcgccacgaaccgcgtttcccagagctggcggagctctcagaaccccacgctcagctgctacagcgccgccttgacttcctcgccaaacacattttgcactgcaaggccaagtacggccgccggcccggggacttaataacactccgaggaagagagggactggggtatgaatgatgggggttagggctgtcttctctgccacgggcaacgaccaaccagccaacgcccacccaatggtctgatcgccaagctgtctaaaaaaaaaaattgtgtgttacccacttgcccctttcttttcatgccaggctgtttcctttccaagttctggaagggcaaactcaccgaggcgagaagtgtaacattccctccacctagcttataaaaagagttctgtgcttgtgtggagattggatccgaagaaactggctgctggggtttggcccgggaatggcagtctctttgggagatgcagtccatttttgtcgccccctccaccccttcccaaaaaggaaaacttccgtttgagctaattcctctatttcctgaactcctatcattttggcctgaaggctatgaattcaggactagttataagcagagtcaataatgaatttcttcctgcgtctcccaattgaccaaaattgacaatgatgatgttcaccagagggggaaaaaaatcagtttcatgcactttactttttttaattttaattttttagtaagaaaacttttttatttgacagaatttgccttctgtgtgtatatatgtgcataaattgtgctgtaaatagactaaacaaacttatatttcaataaaagggagtttaaaatttaaaaaaaaaaaaaaaaaaaaaaaagggcggccgc

SEQ ID NO: 34 - protein, 450 aa, Mus musculus
MGRKMRGAAAAAGLWLLALSSLLTLWGGLLPPRTELPASRPPEDRLPPHPIQSGGPAPEPRFPLPPPLVWDARGGSLKTFRALLTLAAGADNPPRRHQDDRGRHEPSGLSWPEERRAVHGGVFWSRGLEEQVPRGFSEAQAAAWLEVARGARVVALDRGGCGRSSNRLARFADGTRACVRYGINPEQIQGEALSYYLARLLGLQRHVPPLALARVEARGAQWVQVQEELRTAHWTEGSVVSLTRWLPNLTDVVVPEPWRSEDGRLRPLRDAGGELTNLSQAELVDLVQWTDLILFDYLTANFDRLVSNLFSLQWDPRVMHRATSNLHRGPGGALVFLDNEAGLVHGYRVAGMWDKYNEPLLQSVCVFRERTARRVLELHRGQDAAARLLRLYSRHEPRFPELAELSEPHAQLLQRRLDFLAKHILHCKAKYGRRPGDLITLRGREGLGYE

SEQ ID NO: 35 - DNA, 1350 bp, Mus musculus
atggggaggaagatgcggggcgccgccgccgccgcggggctctggctgctggctttgagctcgctgctgacgctgtggggaggactcctgccaccgcggaccgagctgccagcctcccggccgcccgaagatcgactccctccgcatccgatccagagtggcggccccgcgcccgagccgcgattccctctgcccccgcccctagtatgggacgcccgcggcggctccctgaaaactttccgggcgctgctcaccctggcggccggcgcggataacccgcctaggaggcaccaggacgaccgcgggcggcacgagccctccgggctgtcctggccagaggagcgcagggcggtgcacgggggcgtcttctggagccgcggcctggaggagcaggttccccggggcttttccgaagcccaagcagcagcgtggctggaggtggcacggggtgctcgggtggtggctctggatcgcgggggctgcggacgcagttccaaccgcctagcccgctttgccgacggcacccgtgcctgtgtacgctacggcatcaacccagagcagatacagggcgaggccctgtcctactaccttgcgcgcctgctgggcctccagcgccacgtgccgccgctggcactggctcgggtggaggctcggggcgcgcagtgggtgcaggtgcaggaggagctgcgcaccgcgcactggaccgagggcagcgtggtgagcctgacgcgctggctgcctaacctcaccgacgtggtggtgcccgagccctggcgatcagaggacggccgtctgcggcccctgcgcgacgccgggggcgagctgaccaacctcagccaggcggagctggtggacttggtacaatggaccgatctgatcctcttcgattacctgacggccaactttgaccggcttgtgagcaacctcttcagcttacagtgggacccacgcgttatgcaccgcgctacgagcaacctgcaccgaggaccaggaggggcgttggtctttctggacaatgaggcgggcttggtgcacggctaccgggtagccggcatgtgggacaagtataacgaaccgctgctacagtcggtgtgtgtattccgagagcggactgctaggcgcgtcttggagctgcaccggggtcaggacgcggcggcccggttgctgcgcctctacagtcgccacgaaccgcgtttcccagagctggcggagctctcagaaccccacgctcagctgctacagcgccgccttgacttcctcgccaaacacattttgcactgcaaggccaagtacggccgccggcccggggacttaataacactccgaggaagagagggactggggtatgaa

SEQ ID NO: 36 - protein, 484 aa, Mus musculus
APEPASSRARLAARTRAPGLGLKRRRWHRRAGRSMGRKMRGAAAAAGLWLLALSSLLTLWGGLLPPRTELPASRPPEDRLPPHPIQSGGPAPEPRFPLPPPLVWDARGGSLKTFRALLTLAAGADNPPRRHQDDRGRHEPSGLSWPEERRAVHGGVFWSRGLEEQVPRGFSEAQAAAWLEVARGARVVALDRGGCGRSSNRLARFADGTRACVRYGINPEQIQGEALSYYLARLLGLQRHVPPLALARVEARGAQWVQVQEELRTAHWTEGSVVSLTRWLPNLTDVVVPEPWRSEDGRLRPLRDAGGELTNLSQAELVDLVQWTDLILFDYLTANFDRLVSNLFSLQWDPRVMHRATSNLHRGPGGALVFLDNEAGLVHGYRVAGMWDKYNEPLLQSVCVFRERTARRVLELHRGQDAAARLLRLYSRHEPRFPELAELSEPHAQLLQRRLDFLAKHILHCKAKYGRRPGDLITLRGREGLGYE

SEQ ID NO: 37 - DNA, 1384 bp, Rattus norvegicus
  CDS (1)...(507)
gagctggtggacctggtacaatggaccgatctgatcctcttcgattacctgacagccaacttcgaccggcttgtaagcaacctcttcagcttacagtgggacccacgcgttatgcaccgcgctacaagcaacctgcaccgaggaccaggaggggcgttggtctttctggacaatgaggcgggcctggtgcacgggtaccgggtagccggcatgtgggacaagtataacgaaccgctgctacagtcggtgtgtgtattccgagagcggactgctaggcgcgtcttggagctgcaccggggtcaggacgcggcagcccggctcctgcgcctctacagtcgccacgaaccgcgtttcccagagctggcggagctcgcagacccccacgctcagctgctacagcgccgccttgacttcctcgccaaacacattttgcactgtaaggccaagtacggccgccgacccggggacttaataacactccgaggaagagagggactgggttatgaatgatgcgggttagggctgtcttttctgccacgggctatgaccaaccggccaacgcccacccagtggtctgaataccaagctgtctaaaaacttgtgtgttacccacttgcccctttcttttcatgccaggctgtttcctttccaagttctggaagggcaaactcaccgaggcaagaagtgtaacattccctccacctagcttataaaaaggaatctgtgcttgtgtggagattggatccgaagaaactggctgctggggtttggcccctgaatggcagtctctttgggagatgcagtccattcttgcccccccccttcccaaaaaaggaaaacttccgtttgagctaattcctctatttcctactaaacacagcgccggtggccccagagcaggctgtgacaagggctggtggagcccccttcctgtgttctctctttgttccagcgcacgatggtgagataactgttccaagctgagggacagctctggataggacaaagagagcaagacttccccgcccctgggagccctacagaccctgacagtttgtctgacccattcctgaacccctgtcattttagcctgaaggctatgaattcggaactagttataagcagagtcaataatgaatttcttcccgcatctcccaattgaccaaaattgacaatgatgatgttcaccagaagaaagaaaaaaatcagtttcatgcactttactttttttaattttaattttttagtaagaaaacttttttatttgacagaatttgccttctgtgtatatatgtgcataaatcgtggtgtaaatagactaaacaaacttatatttcaataaaagggagtttaaaatttaaaaaaaaaaaaaaagggcggccgc

SEQ ID NO: 38 - protein, 169 aa, Rattus norvegicus
ELVDLVQWTDLILFDYLTANFDRLVSNLFSLQWDPRVMHRATSNLHRGPGGALVFLDNEAGLVHGYRVAGMWDKYNEPLLQSVCVFRERTARRVLELHRGQDAAARLLRLYSRHEPRFPELAELADPHAQLLQRRLDFLAKHILHCKAKYGRRPGDLITLRGREGLGYE

SEQ ID NO: 39 - protein, 473 aa, Drosophila melanogaster
LPEEQIQMVAVEPLSTYRVEFIKETDELSAEQVFRNAFHLEQDKDAPDSMVVKKLDTNDGSIKEFHVQRTASGRYRKGPERRMSKKMPERVQPQETSRSPTTSPTNPTSEHQAGLIEEDVYWGPTVEQALPKGFAAKDQVSWERFVGEQGRVVRLEQGCGRMQNRMVVFADGTRACAPYRQNTDQIQGEIFSYYLGQLLNISNLAPSAATVVDTSTPNWRAALGDITQAQWKERRPVVLTRWLSDLEPAGIPQPFQPLERHLNKHDVWNLTRHMQSERQAQSQPHGLLKRLGAASSPGSAHQSNAIEETGTGTETANGALVQRLIELAQWSDLIVFDYLIANLDRVVNNLYNFQWNADIMAAPAHNLARQSASQLLVFLDNESGLLHGYRLLKKYEAYHSLLLDNLCVFRRPTIDALRRLRAAGAGRRLRDLFERTTSAGVRDVLPSLPDKSVKILVERIDRVLGQVQKCQGS

SEQ ID NO: 40 - DNA, 1440 bp, Homo sapiens
cggccgcgatggggccgaagcgcccgaagccccggagcccacaaactgccgggcccgcctcgccgccgggacccgggtgcctgggctcggcttgaagcggcggcggcgcaccggcacagccgcgggagcatgggcaggaggatgcggggcgccgccgccaccgcggggctctggctgctggcgctgggctcgctgctggcgctgtggggagggctcctgccgccgcggaccgagctgcccgcctcccggccgcccgaagaccgactcccacggcgcccggcccggagcggcggccccgcgcccgcgcctcgcttccctctgcccccgcccctggcgtgggacgcccgcggcggctccctgaaaactttccgggcgctgctcaccctggcggccggcgcggacggcccgccccggcagtcccggagcgagcccaggtggcacgtgtcagccaggcagccccggccggaggagagcgccgcggtgcacgggggcgtcttctggagccgcggcctggaggagcaggtgcccccgggcttttcggaggcccaggcggcggcgtggctggaggcggctcgcggcgcccggatggtggccctggagcgcgggggttgcgggcgcagctccaaccgactggcccgttttgccgacggcacccgcgcctgcgtgcgctacggcatcaacccggagcagattcagggcgaggccctgtcttactatctggcgcgcctgctgggcctccagcgccacgtgccgccgctggcactggctcgggtggaggctcggggcgcgcagtgggcgcaggtgcaggaggagctgcgcgctgcgcactggaccgagggcagcgtggtgagcctgacacgctggctgcccaacctcacggacgtggtggtgcccgcgccctggcgctcggaggacggccgtctgcgccccctccgggatgccgggggtgagctggccaacctcagccaggcggagctggtggacctagtacaatggaccgacttaatccttttcgactacctgacggccaacttcgaccggctcgtaagcaacctcttcagcctgcagtgggacccgcgcgtcatgcagcgtgccaccagcaacctgcaccgcggtccgggcggggcgctggtctttctggacaatgaggcgggcttggtgcacggctaccgggtagcaggcatgtgggacaagtataacgagccgctgttgcagtcagtgtgcgtgttccgcgagcggaccgcgcggcgcgtcctggagctgcaccgcggacaggacgccgcggcccggctgctgcgcctctaccggcgccacgagcctcgcttccccgagctggccgcccttgcagacccccacgctcagctgctacagcgccgcctcgacttcctcgccaagcacattttgcactgtaaggccaagtacggccgccggtctgggact

SEQ ID NO: 41 - DNA, 1452 bp, Mus musculus
gccccggagccagctagcagccgggcccgcctcgccgcccgcacccgggcgcccgggcttggcttgaagcggcggcggtggcaccggcgcgccggcaggagcatggggaggaagatgcggggcgccgccgccgccgcggggctctggctgctggctttgagctcgctgctgacgctgtggggaggactcctgccaccgcggaccgagctgccagcctcccggccgcccgaagatcgactccctccgcatccgatccagagtggcggccccgcgcccgagccgcgattccctctgcccccgcccctagtatgggacgcccgcggcggctccctgaaaactttccgggcgctgctcaccctggcggccggcgcggataacccgcctaggaggcaccaggacgaccgcgggcggcacgagccctccgggctgtcctggccagaggagcgcagggcggtgcacgggggcgtcttctggagccgcggcctggaggagcaggttccccggggcttttccgaagcccaagcagcagcgtggctggaggtggcacggggtgctcgggtggtggctctggatcgcgggggctgcggacgcagttccaaccgcctagcccgctttgccgacggcacccgtgcctgtgtacgctacggcatcaacccagagcagatacagggcgaggccctgtcctactaccttgcgcgcctgctgggcctccagcgccacgtgccgccgctggcactggctcgggtggaggctcggggcgcgcagtgggtgcaggtgcaggaggagctgcgcaccgcgcactggaccgagggcagcgtggtgagcctgacgcgctggctgcctaacctcaccgacgtggtggtgcccgagccctggcgatcagaggacggccgtctgcggcccctgcgcgacgccgggggcgagctgaccaacctcagccaggcggagctggtggacttggtacaatggaccgatctgatcctcttcgattacctgacggccaactttgaccggcttgtgagcaacctcttcagcttacagtgggacccacgcgttatgcaccgcgctacgagcaacctgcaccgaggaccaggaggggcgttggtctttctggacaatgaggcgggcttggtgcacggctaccgggtagccggcatgtgggacaagtataacgaaccgctgctacagtcggtgtgtgtattccgagagcggactgctaggcgcgtcttggagctgcaccggggtcaggacgcggcggcccggttgctgcgcctctacagtcgccacgaaccgcgtttcccagagctggcggagctctcagaaccccacgctcagctgctacagcgccgccttgacttcctcgccaaacacattttgcactgcaaggccaagtacggccgccggcccggggacttaataacactccgaggaagagagggactggggtatgaa

SEQ ID NO: 42 - protein, 409 aa, Homo sapiens
LLPPRTELPASRPPEDRLPRRPARSGGPAPAPRFPLPPPLAWDARGGSLKTFRALLTLAAGADGPPRQSRSEPRWHVSARQPRPEESAAVHGGVFWSRGLEEQVPPGFSEAQAAAWLEAARGARMVALERGGCGRSSNRLARFADGTRACVRYGINPEQIQGEALSYYLARLLGLQRHVPPLALARVEARGAQWAQVQEELRAAHWTEGSVVSLTRWLPNLTDVVVPAPWRSEDGRLRPLRDAGGELANLSQAELVDLVQWTDLILFDYLTANFDRLVSNLFSLQWDPRVMQRATSNLHRGPGGALVFLDNEAGLVHGYRVAGMWDKYNEPLLQSVCVFRERTARRVLELHRGQDAAARLLRLYRRHEPRFPELAALADPHAQLLQRRLDFLAKHILHCKAKYGRRSGT

SEQ ID NO: 43 - DNA, 384 bp, Mus musculus
  CDS (18)...(206)
catccttcagcagcagcatgaagctaggagccttccttctcttggtgtccctcatcaccctcagcctagaggtacaggagctgcaggctgcagtgagacctctgcagcttttaggcacctgtgctgagctctgccgtggtgactgggactgtgggccagaggaacaatgtgtcagtattggatgcagtcacatctgtactacaaaccaaaaacagcttctacctggaaaaaaaaatgtgtctgtttggagctctgtgaccaagaaaacagttgaaaatggaggccatgtatggagattacaagcagcacagtggagtgggacaaggagttgtttcttttaataaatcattaatgtaaaagtctcaaaaaaaaaaaaaaaaaaaa

SEQ ID NO: 44 - protein, 63 aa, Mus musculus
  SIGNAL (1)...(24)
MKLGAFLLLVSLITLSLEVQELQAAVRPLQLLGTCAELCRGDWDCGPEEQCVSIGCSHICTTN

SEQ ID NO: 45 - DNA, 189 bp, Mus musculus
atgaagctaggagccttccttctcttggtgtccctcatcaccctcagcctagaggtacaggagctgcaggctgcagtgagacctctgcagcttttaggcacctgtgctgagctctgccgtggtgactgggactgtgggccagaggaacaatgtgtcagtattggatgcagtcacatctgtactacaaac

SEQ ID NO: 46 - DNA, 489 bp, Homo sapiens
  CDS (23)...(205)
gaattcggcacgaggcagcaacatgaagttggcagccttcctcctcctggtgatcctcatcatcttcagcctagaggtacaagagcttcaggctgcaggagaccggcttttgggtacctgcgtcgagctctgcacaggtgactgggactgcaaccccggagaccactgtgtcagcaatgggtgtggccatgagtgtgttgcagggtaaggacaggtaaaaacaccaggccctccctgctttctgaaacgttgttcagtctagatgaagagttatcttaaggatcatctttccctaagatcgtcatcccttcctggagttcctatcttccaagatgtgactgtctggagttccttgactaggaagatggatgaaaacagcaagcctgtggatggagactacaggggatatgggaggcagggaagaggggttgtttcttttaataaatcatcattgttaaaagcaaaaaaaaaaaaaaaaaaaaaaaaaaa

SEQ ID NO: 47 - DNA, 489 bp, Homo sapiens
gaattcggcacgaggcagcaacatgaagttggcagccttcctcctcctggttatcctcatcatcttcagcctagaggtacaagagcttcaggctgcaggagaccggcttttgggtacctgcgtcgagctctgcacaggtgactgggactgcaaccccggagaccactgtgtcagcaatgggtgtggccatgagtgtgttgcagggtaaggacaggtaaaaacaccaggccctccctgctttctgaaacgttgttcagtctagatgaagagttatcttaaggatcatctttccctaagatcgtcatcccttcctggagttcctatcttccaagatgtgactgtctggagttccttgactaggaagatggatgaaaacagcaagcctgtggatggagactacaggggatatgggaggcagggaagaggggttgtttcttttaataaatcatcattgttaaaagcaaaaaaaaaaaaaaaaaaaaaaaaaaa

SEQ ID NO: 48 - DNA, 489 bp, Homo sapiens
gaattcggcacgaggcagcaacatgaagttggcagccttcctcctcctggtcatcctcatcatcttcagcctagaggtacaagagcttcaggctgcaggagaccggcttttgggtacctgcgtcgagctctgcacaggtgactgggactgcaaccccggagaccactgtgtcagcaatgggtgtggccatgagtgtgttgcagggtaaggacaggtaaaaacaccaggccctccctgctttctgaaacgttgttcagtctagatgaagagttatcttaaggatcatctttccctaagatcgtcatcccttcctggagttcctatcttccaagatgtgactgtctggagttccttgactaggaagatggatgaaaacagcaagcctgtggatggagactacaggggatatgggaggcagggaagaggggttgtttcttttaataaatcatcattgttaaaagcaaaaaaaaaaaaaaaaaaaaaaaaaaa

SEQ ID NO: 49 - DNA, 489 bp, Homo sapiens
gaattcggcacgaggcagcaacatgaagttggcagccttcctcctcctggtaatcctcatcatcttcagcctagaggtacaagagcttcaggctgcaggagaccggcttttgggtacctgcgtcgagctctgcacaggtgactgggactgcaaccccggagaccactgtgtcagcaatgggtgtggccatgagtgtgttgcagggtaaggacaggtaaaaacaccaggccctccctgctttctgaaacgttgttcagtctagatgaagagttatcttaaggatcatctttccctaagatcgtcatcccttcctggagttcctatcttccaagatgtgactgtctggagttccttgactaggaagatggatgaaaacagcaagcctgtggatggagactacaggggatatgggaggcagggaagaggggttgtttcttttaataaatcatcattgttaaaagcaaaaaaaaaaaaaaaaaaaaaaaaaaa

SEQ ID NO: 50 - DNA, 183 bp, Homo sapiens
atgaagttggcagccttcctcctcctggtgatcctcatcatcttcagcctagaggtacaagagcttcaggctgcaggagaccggcttttgggtacctgcgtcgagctctgcacaggtgactgggactgcaaccccggagaccactgtgtcagcaatgggtgtggccatgagtgtgttgcaggg

SEQ ID NO: 51 - DNA, 183 bp, Homo sapiens
atgaagttggcagccttcctcctcctggttatcctcatcatcttcagcctagaggtacaagagcttcaggctgcaggagaccggcttttgggtacctgcgtcgagctctgcacaggtgactgggactgcaaccccggagaccactgtgtcagcaatgggtgtggccatgagtgtgttgcaggg

SEQ ID NO: 52 - DNA, 183 bp, Homo sapiens
atgaagttggcagccttcctcctcctggtcatcctcatcatcttcagcctagaggtacaagagcttcaggctgcaggagaccggcttttgggtacctgcgtcgagctctgcacaggtgactgggactgcaaccccggagaccactgtgtcagcaatgggtgtggccatgagtgtgttgcaggg

SEQ ID NO: 53 - DNA, 183 bp, Homo sapiens
atgaagttggcagccttcctcctcctggtaatcctcatcatcttcagcctagaggtacaagagcttcaggctgcaggagaccggcttttgggtacctgcgtcgagctctgcacaggtgactgggactgcaaccccggagaccactgtgtcagcaatgggtgtggccatgagtgtgttgcaggg

SEQ ID NO: 54 - protein, 61 aa, Homo sapiens
  SIGNAL (1)...(24)
MKLAAFLLLVILIIFSLEVQELQAAGDRLLGTCVELCTGDWDCNPGDHCVSNGCGHECVAG

SEQ ID NO: 55 - DNA, 471 bp, Mus musculus
  CDS (37)...(264)
aaattatttcatgagttcagccgagccagagccaacatgaagacagccacagtcttgtttctggtggctttgatcactgtggggatgaacactacctatgtagtgtcttgccccaaagaatttgaaaaacctggagcttgtcccaagccttcaccagaaagtgttggaatttgtgttgatcaatgctcaggagatggatcctgccctggcaacatgaagtgctgtagcaatagctgtggtcatgtctgcaaaactcctgtcttttaaatggttgacagccatgtggaagatggattcaatcttcataaacatgaatgatggccagccccagaagatttcttctgaattcacagagcctgtgcttggctacttcctagccctagaattgcattcttggacaaggaagatctatattgtggtgacaatgccctaatatgtctgtgtccaaaataaactacccttagcattcag

SEQ ID NO: 56 - protein, 76 aa, Mus musculus
  SIGNAL (1)...(17)
MKTATVLFLVALITVGMNTTYVVSCPKEFEKPGACPKPSPESVGICVDQCSGDGSCPGNMKCCSNSCGHVCKTPVF

SEQ ID NO: 57 - DNA, 228 bp, Mus musculus
atgaagacagccacagtcttgtttctggtggctttgatcactgtggggatgaacactacctatgtagtgtcttgccccaaagaatttgaaaaacctggagcttgtcccaagccttcaccagaaagtgttggaatttgtgttgatcaatgctcaggagatggatcctgccctggcaacatgaagtgctgtagcaatagctgtggtcatgtctgcaaaactcctgtcttt

SEQ ID NO: 58 - protein, 74 aa, Mus musculus
MKTATVFVLVALIFMTMTTAWALSNPKEKPGACPKPPPRSFGTCDERCTGDGSCSGNMKCCSNGCGHACKPPVF

SEQ ID NO: 59 - protein, 60 aa, Rattus norvegicus
MNITYALFSPTKLEKPGKCPKNPPRSIGTCVELCSGDQSCPNIQKCCSNGCGHVCKSPVF

SEQ ID NO: 60 - protein, 53 aa, Artificial Sequence
  clone
SSGRREVQELQAAVRPLQLLGTCAELCRGDWDCGPEEQCVSIGCSHICTSAAT

SEQ ID NO: 61 - protein, 131 aa, Mus musculus
MKSCGLLPFTVLLALGILAPWTVEGGKNDAIKIGACPAKKPAQCLKLEKPQCRTDWECPGKQRCCQDACGSKCVNPVPIRKPVWRKPGRCVKTQARCMMLNPPNVCQRDGQCDGKYKCCEGICGKVCLPPM

SEQ ID NO: 62 - DNA, 501 bp, Artificial Sequence
  clone
gaattcggcacgaggcagcaacatgaagttggcagccttcctcctcctgtgatcctcatcatcttcagcctagaggtacaagagcttcaggctgcaggagaccggcttttgggtacctgcgtcgagctctgcacaggtgactgggactgcaaccccggagaccactgtgtcagcaatgggtgtggccatgagtgtgttgcagggtaaggacaggtaaaaacaccaggccctccctgctttctgaaacgttgttcagtctagatgaagagttatcttaaggatcatctttccctaagatcgtcatcccttcctggagttcctatcttccaagatgtgactgtctggagttccttgactaggaagatggatgaaaacagcaagcctgtggatggagactacaggggatatgggaggcagggaagaggggttgtttcttttaataaatcatcattgttaaaagcaaaaaaaaaaaaaaaaaaaaaaaaaaatggttgcggccgc

SEQ ID NO: 63 - protein, 39 aa, Mus musculus
AVRPLQLLGTCAELCRGDWDCGPEEQCVSIGCSHICTTN

SEQ ID NO: 64 - protein, 37 aa, Homo sapiens
AGDRLLGTCVELCTGDWDCNPGDHCVSNGCGHECVAG

SEQ ID NO: 65 - DNA, 18 bp, Artificial Sequence
  oligonucleotide for PCR
tcgtcgtacttcgatcct

SEQ ID NO: 66 - DNA, 18 bp, Artificial Sequence
  oligonucleotide for PCR
tacttcgatcctcggagg

SEQ ID NO: 67 - protein, 59 aa, Mus musculus
NTTYVVSCPKEFEKPGACPKPSPESVGICVDQCSGDGSCPGNMKCCSNSCGHVCKTPVF

SEQ ID NO: 68 - protein, 8 aa, Artificial Sequence
  flag epitope
DYKDDDDK